(19) United States
(12) Patent Application Publication
(10) Pub. No.: US 2019/0233851 A1
(43) Pub. Date: Aug. 1, 2019

(54) METHODS AND MATERIALS FOR THE BIOSYNTHESIS OF COMPOUNDS OF FATTY ACID METABOLISM AND RELATED COMPOUNDS

(71) Applicant: INVISTA NORTH AMERICA S.A.R.L., Wilmington, DE (US)

(72) Inventors: Jonathan KENNEDY, Redcar (GB); Alexander Brett FOSTER, Redcar (GB)

(21) Appl. No.: 16/264,782

(22) Filed: Feb. 1, 2019

Related U.S. Application Data
(60) Provisional application No. 62/711,826, filed on Jul. 30, 2018.
(60) Provisional application No. 62/625,031, filed on Feb. 1, 2018.

(51) Int. Cl. C12P 7/42; C12N 9/16; C12N 9/02; C12N 9/88; C12N 9/00; C12N 15/52; C12R 1/01

(52) U.S. Cl. CPC C12P 7/42; C12N 9/16; C12N 9/001; C12N 9/88; C12N 9/93; C12Y 602/01003; C12R 1/01; C12Y 301/02007; C12Y 103/01039; C12Y 401/99005; C12N 15/52

(57) ABSTRACT

Methods and materials for the production of compounds involved in fatty acid metabolism, and/or derivatives thereof and/or compounds related thereto are provided. Also provided are products produced in accordance with the methods and materials of the present invention.

This patent application claims the benefit of priority from U.S. Provisional Application Ser. No. 62/711,826 filed Jul. 30, 2018 and U.S. Provisional Application Ser. No. 62/625,031, filed Feb. 1, 2018, the contents of each of which are herein incorporated by reference in their entirety.

### FIELD

The present invention relates to biosynthetic methods and materials for the production of compounds involved in fatty acid metabolism, and/or derivatives thereof and/or other compounds related thereto. The present invention comprises products biosynthesized, or otherwise encompassed, by these biosynthetic methods and materials.

Replacement of traditional chemical production processes relying on, for example fossil fuels and/or potentially toxic chemicals, with environmentally friendly (e.g., green chemicals) and/or “cleantech” solutions is being considered, including work to identify building blocks suitable for use in the manufacturing of such chemicals. See, “Conservative evolution and industrial metabolism in Green Chemistry”, Green Chem., 2018, 20, 2171-2191.

Fatty acids are an integral component of all living systems, being essential for biological membranes.

The major precursor of fatty acids, malonyl-CoA, is formed from the carboxylation of acetyl-CoA by acetyl-CoA carboxylase (ACC). The malonyl group is then transferred from CoA to ACP by FabD. Fatty acid synthesis is then initiated by the decarboxylative condensation of acetyl-CoA and malonyl-ACP to form acetoacetyl-ACP. Successive rounds of ketoreduction, dehydration and enoyl reduction result in the formation of butyryl-ACP. The cycle is then repeated by the successive addition and reduction of malonyl units until the long chain acyl-ACP (typically C16-18) enters glycerol(phospho)lipid metabolism (Beld et al. Mol Biosyst. 2015 January; 11(1):38-59).

Biotechnological manipulation of microbial fatty acid metabolism has been investigated as a potential source of biofuels and other oleochemicals (Tee et al. Biotechnol Bioeng. 2014 May; 111(5):849-57; Gronenburg et al. Curr Opin Chem Biol. 2013 June; 17(3):462-71).

Some fatty acid biochemical pathways have been known and are described herein, in FIG. 1.

Expression of polypeptides having thioesterase (TE) activity has been used to convert fatty acyl-ACPs and result in the formation of free fatty acids (Lennen and Pfleger, Trends Biotechnol. 2012 30(12):659-67; Chen et al., PeerJ 2015 3:e1468; DOI 10.7717/peerj.1468). The chain length of the resultant fatty acids is dependent upon the specificity of the TE used (Jing et al. BMC Biochemistry 2011 12.1:44). In E. coli there is feedback regulation at the level of long chain acyl-ACP (Heath, R. J. & Rock, C. O. Journal of Biological Chemistry 1996 271(18): 10966-11000). Expression of a TE can increase fatty acid titers (Jing et al. supra).

Expression of acyl-ACP reductase and aldehyde decarbonylase from cyanobacteria in E. coli results in the conversion of acyl-ACPs to alka(e)nes in a two step process (Schirmer et al. Science 2010 329(5991):559-62). This pathway has been introduced into C. necator with titers of 670 mg/L total hydrocarbon reported, with pentadecane being the major alkane product (Crepin et al. Metab Eng. 2016 37:92-101).

Expression of fatty acyl-CoA reductase (FAR) has been reported to result in the conversion of fatty acyl-CoAs to fatty aldehydes and fatty alcohols (Metz et al. Plant Physiology 2000 122.3:635-644). Some CoA FAR enzymes have been demonstrated to function with fatty acyl-ACPs as substrates although the preferred substrate is acyl-CoA (Hofvander et al. FEBS letters 2011 585(22):3538-3543). Although it has been reported some FAR enzymes have been demonstrated to prefer acyl-ACPs (Shi et al. The Plant Cell 2011 tpc-111).

Highest titers have generally been observed in bacterial strains co-expressing a TE and an acyl-CoA ligase (see FIG. 1) (Youngquist et al. Metab Eng. 2013 177-86; U.S. Pat. No. 8,883,467 B2).

Overexpression of acetyl-CoA carboxylase (acc) to improve fatty acid production in E. coli has been disclosed (Davis et al. The Journal of Biological Chemistry 2000 275:28593-28598). C. necator is able to actively degrade fatty acids via β-oxidation pathways (Brigham et al. J Bacteriol. 2010 October; 192(20):5454-64; Reidel et al. Applied Microbiology and Biotechnology 2014 98.4:1469-1483). Deletion of β-oxidation pathways in C. necator have been used to study fatty acid catabolism (Brigham et al., supra) to improve production of methyl ketones (Muller et al. Appl Environ Microbiol. 2013 79(14):4433-92013).

Biosynthetic materials and methods, including improved organisms having increased production of compounds involved in fatty acid metabolism, derivatives thereof and compounds related thereto are needed.

### SUMMARY OF THE INVENTION

An aspect of the present invention relates to a process for biosynthesis of compounds involved in fatty acid metabolism, and/or derivatives thereof and/or compounds related thereto. The processes of the present invention comprise obtaining an organism capable of producing compounds involved in fatty acid metabolism and derivatives and compounds related thereto, altering the organism, and producing more compounds involved in fatty acid metabolism and derivatives and compounds related thereto in the altered organism as compared to the unaltered organism. In one nonlimiting embodiment, the organism is C. necator or an organism with one or more properties similar thereto. In one nonlimiting embodiment, the organism is altered by inserting a non-natural pathway to intercept fatty acyl-ACP intermediates. In one nonlimiting embodiment, a thioesterase is inserted to generate free fatty acids. In one nonlimiting embodiment, a fatty acyl-CoA reductase is inserted to generate fatty alcohols. In one nonlimiting embodiment, an acyl-ACP reductase, an aldehyde decarbonylase, an oxidoreductase and/or an acyl-CoA synthetase is inserted.

In one nonlimiting embodiment, the thioesterase comprises E. coli ′tesA (SEQ ID NO:19), a truncated version of the full tesA lacking the N-terminal signal peptide, a thioesterase selected from SEQ ID NO: 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 or 81 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 or 81 or a functional fragment thereof. In one nonlimiting embodiment, the thioesterase is encoded by a nucleic acid sequence comprising E. coli ′tesA (SEQ ID NO:20), a nucleic acid sequence selected from SEQ ID NO: 22, 24, 26, 28, 30, 32, 34, 36, 38, 40, 42, 44, 46, 48, 50, 52, 54, 56, 58, 60, 62, 64, 66, 68, 70, 72, 74, 76, 80 or 82 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 20, 22, 24, 26, 28, 30, 32, 34, 36, 38, 40, 42, 44, 46, 48, 50, 52, 54, 56, 58, 60, 62, 64, 66, 68, 70, 72, 74, 76, 80 or 82 or a functional fragment thereof.

In one nonlimiting embodiment, the fatty acyl-CoA reductase is from Bermanella marisrubri or Marinobacter algicola and comprises SEQ ID NO: 9 or 11 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 9 or 11 or a functional fragment thereof. In one nonlimiting embodiment, the fatty acyl-CoA reductase is from Bermanella marisrubri or Marinobacter algicola and is encoded by a nucleic acid sequence comprising SEQ ID NO: 10 or 12 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 10 or 12 or a functional fragment thereof.

In one nonlimiting embodiment, the acyl-ACP reductase is from Synechococcus and comprises SEQ ID NO:1 or a polypeptide with similar enzymatic activities exhibiting at least about 50%, 60% 70%, 75%, 80%, 85%, 90%, 91%, 92%, 93%, 94%, 95%, 96%, 97%, 98%, 99% or 99.5% sequence identity to an amino acid sequence set forth in SEQ ID NO: 1 or a functional fragment thereof. In one nonlimiting embodiment, the acyl-ACP reductase is from Synechococcus and is encoded by a nucleic acid sequence comprising SEQ ID NO:2 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or a functional fragment thereof.

In one nonlimiting embodiment, the aldehyde decarbonylase is from Synechococcus and comprises SEQ ID NO:3 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 3 or a functional fragment thereof. In one nonlimiting embodiment, the aldehyde decarbonylase is from Synechococcus and is encoded by a nucleic acid sequence comprising SEQ ID NO:4 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 4 or a functional fragment thereof.

In one nonlimiting embodiment, the oxidoreductase is from E. coli and comprises SEQ ID NO:5 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 5 or a functional fragment thereof. In one nonlimiting embodiment, the oxidoreductase is from E. coli and is encoded by a nucleic acid sequence comprising SEQ ID NO:6 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 6 or a functional fragment thereof.

In one nonlimiting embodiment, the acyl-CoA synthetase is from E. coli and comprises SEQ ID NO:7 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 7 or a functional fragment thereof. In one nonlimiting embodiment, the acyl-CoA synthetase is from E. coli and is encoded by a nucleic acid sequence comprising SEQ ID NO:8 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 8 or a functional fragment thereof.

In one nonlimiting embodiment, the nucleic acid sequence is codon optimized for C. necator.

In one nonlimiting embodiment, the organism is further altered to delete one or more enzymes of the β-oxidation pathway.

In one nonlimiting embodiment, the fatty acid is pimelic acid and the organism is further altered to delete one or more enzymes which activate pimelate. For example, one or more genes selected from A3350-51 (acyl-CoA ligase and transport genes), A1519-20 (acyl-CoA ligase and transport genes), and B1446-9 (acyl-CoA transferase, transport and regulatory gene) can be deleted. In one nonlimiting embodiment, the fatty acid is pimelic acid and the organism is further altered to inhibit acyl-CoA dehydrogenase. For example, one or more genes selected from A2818 (glutaryl-CoA dehydrogenase gene), B2555 (acyl-CoA dehydrogenase gene) and A0814-16 (electron transfer and acyl-CoA dehydrogenase genes) can be deleted. In one nonlimiting embodiment, the fatty acid is pimelic acid and the organism is further altered to delete a cluster selected from A0459-0464 0-oxidation cluster 1) and A1526-1531 (β-oxidation cluster 2).

In one nonlimiting embodiment, the fatty acid is adipic acid and the organism is further altered by deleting an adipic acid specific operon. In one nonlimiting embodiment, the adipic acid specific operon is B0198-202 (acyl-CoA transferase, thiolase, dehydrogenase and transport). In one nonlimiting embodiment, the fatty acid is adipic acid and the organism is further altered to delete one or more enzymes which activate adipate. For example, B1446-9 (acyl-CoA transferase, transport and regulatory gene) can be deleted. In one nonlimiting embodiment, the fatty acid is adipic acid and the organism is further altered to inhibit acyl-CoA dehydrogenase. For example, one or more genes selected from B2555 (acyl-CoA dehydrogenase gene), A1526-1531 (β-oxidation cluster 2), A2818 (glutaryl-CoA dehydrogenase gene), A0814-16 (electron transfer and acyl-CoA dehydrogenase genes) or A1067/68 (acyl-CoA dehydrogenase genes) can be deleted. In one nonlimiting embodiment, the fatty acid is adipic acid and the organism is further altered to delete A0459-0464 (β-oxidation cluster 1).

In one nonlimiting embodiment, the organism is further modified to eliminate phaCAB, involved in PHBs production and/or H16-A0006-9 encoding endonucleases thereby improving transformation efficiency.

Another aspect of the present invention relates to an organism altered to produce more compounds involved in fatty acid metabolism and/or derivatives and compounds related thereto as compared to the unaltered organism. In one nonlimiting embodiment, the organism is C. necator or an organism with properties similar thereto. In one nonlimiting embodiment, the organism is altered by inserting a non-natural pathway to intercept fatty acyl-ACP intermediates. In one nonlimiting embodiment, a thioesterase, as disclosed herein, is inserted to generate free fatty acids. In one nonlimiting embodiment, a fatty acyl-CoA reductase, as disclosed herein is inserted to generate fatty alcohols. In one nonlimiting embodiment, an acyl-ACP reductase and/or aldehyde decarbonylase, as disclosed herein, is inserted to generate alka(e)nes.

In one nonlimiting embodiment, the organism is altered with a nucleic acid sequence codon optimized for C. necator.

In one nonlimiting embodiment, the organism is further altered to delete one or more enzymes of the 3-oxidation pathway.

In one nonlimiting embodiment, the fatty acid is pimelic acid and the organism is further altered to delete one or more enzymes which activate pimelate. For example, one or more genes selected from A3350-51 (acyl-CoA ligase and transport genes), A1519-20 (acyl-CoA ligase and transport genes), and B1446-9 (acyl-CoA transferase, transport and regulatory gene) can be deleted. In one nonlimiting embodiment, the fatty acid is pimelic acid and the organism is further altered to inhibit acyl-CoA dehydrogenase. For example, one or more genes selected from A2818 (glutaryl-CoA dehydrogenase gene), B2555 (acyl-CoA dehydrogenase gene) and A0814-16 (electron transfer and acyl-CoA dehydrogenase genes) can be deleted. In one nonlimiting embodiment, the fatty acid is pimelic acid and the organism is further altered to delete a cluster selected from A0459-0464 (β-oxidation cluster 1) and A1526-1531 (β-oxidation cluster 2).

In one nonlimiting embodiment, the fatty acid is adipic acid and the organism is further altered by deleting an adipic acid specific operon. In one nonlimiting embodiment, the adipic acid specific operon is B0198-202 (acyl-CoA transferase, thiolase, dehydrogenase and transport). In one nonlimiting embodiment, the fatty acid is adipic acid and the organism is further altered to delete one or more enzymes which activate adipate. For example, B1446-9 (acyl-CoA transferase, transport and regulatory gene) can be deleted. In one nonlimiting embodiment, the fatty acid is adipic acid and the organism is further altered to inhibit acyl-CoA dehydrogenase. For example, one or more genes selected from B2555 (acyl-CoA dehydrogenase gene), A1526-1531 (β-oxidation cluster 2), A2818 (glutaryl-CoA dehydrogenase gene), A0814-16 (electron transfer and acyl-CoA dehydrogenase genes) or A1067/68 (acyl-CoA dehydrogenase genes) can be deleted. In one nonlimiting embodiment, the fatty acid is adipic acid and the organism is further altered to delete A0459-0464 (β-oxidation cluster 1).

In one nonlimiting embodiment, the organism is further modified to eliminate phaCAB, involved in PHBs production and/or H16-A0006-9 encoding endonucleases thereby improving transformation efficiency.

In one nonlimiting embodiment, the organism is altered to express, overexpress, not express or express less of one or more molecules depicted in FIG. 1, 7 or 8. In one nonlimiting embodiment, the molecule(s) comprise a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to an amino acid sequence corresponding to a molecule(s) depicted in FIG. 1, 7 or 8, or a functional fragment thereof.

Another aspect of the present invention relates to bio-derived, bio-based, or fermentation-derived products produced from any of the methods and/or altered organisms disclosed herein. Such products include compositions comprising at least one bio-derived, bio-based, or fermentation-derived compound or any combination thereof; molded substances obtained by molding the bio-derived, bio-based, or fermentation-derived compositions or compounds, polyamides; and bio-derived, bio-based, or fermentation-derived semi-solids or non-semi-solid streams comprising the bio-derived, bio-based, or fermentation-derived compositions or compounds, molded substances, or any combination thereof.

Another aspect of the present invention relates to a bio-derived, bio-based or fermentation derived product biosynthesized in accordance with the exemplary central metabolism depicted in FIG. 1, 7 or 8.

Another aspect of the present invention relates to exogenous genetic molecules of the altered organisms disclosed herein. In one nonlimiting embodiment, the exogenous genetic molecule comprises a codon optimized nucleic acid sequence encoding one or more enzymes of a non-natural pathway to intercept fatty acyl-ACP intermediates. In one nonlimiting embodiment, the nucleic acid sequence encodes a thioesterase, as disclosed herein, to generate free fatty acids. In one nonlimiting embodiment, the nucleic acid sequence encodes a fatty acyl-CoA reductase, as disclosed herein, to generate fatty alcohols. In one nonlimiting embodiment, the nucleic acid sequence encodes an acyl-ACP reductase and/or aldehyde decarbonylase, as disclosed herein to generate alka(e)nes. Additional nonlimiting examples of exogenous genetic molecules include expression constructs and synthetic operons of one or more enzymes of a non-natural pathway to intercept fatty acyl-ACP intermediates as disclosed herein.

Yet another aspect of the present invention relates to means and processes for use of these means for biosynthesis of compounds involved in fatty acid metabolism, and/or derivatives thereof and/or compounds related thereto.

### BRIEF DESCRIPTION OF THE FIGURES

FIG. 1 is a schematic of biosynthetic routes from the lipid intermediate, fatty acyl-ACP, to fatty acids, fatty alcohols, and alkanes.

FIG. 2 shows free fatty acid levels of thioesterase expressing C. necator strains produced in accordance with the present invention.

FIG. 3 shows results from shake flask production of alkanes in organisms produced in accordance with the present invention.

FIG. 4 shows results from shake flask production of fatty alcohols in organisms expressing FAR genes and organisms expressing AAR plus oxidoreductase produced in accordance with the present invention.

FIG. 5 shows results of alkane production in Ambr15 fermentation. Strain S11 (β-oxidation mutant+AAR/ADO) was fermented in Ambr15 system. Expression from P_{araBAD} was induced with arabinose at 12 hours, and feeding was stopped at 47 hours. Samples for analysis were taken at the times indicated (induction time point, in the growth phase and post feed).

FIG. 6 shows total free fatty acids production in the Ambr15 fermentation run. Strains fermented include EVC (empty vector control)−S21, TESA−S22, and TESA+ACC−S23. Time points included T1=induction time point; T2=12 hours post induction; T3=36 hours.

FIG. 7 shows the active pathway for the degradation of adipic acid in C. necator H16, based on analyses of transcriptomic data.

FIG. 8 shows the active pathway for the degradation of pimelic acid in C. necator H16, based on analyses of transcriptomic data.

### DETAILED DESCRIPTION

The present invention provides processes for biosynthesis of compounds involved in fatty acid metabolism, and/or derivatives thereof, and/or compounds related thereto, as well as synthetic, recombinant organisms altered to increase the biosynthesis of compounds involved in fatty acid metabolism, derivatives thereof and compounds related thereto, exogenous genetic molecules of these altered organisms, and bio-derived, bio-based, or fermentation-derived products biosynthesized or otherwise produced by any of these methods and/or altered organisms.

In the present invention, an organism is engineered and/or redirected to produce compounds involved in fatty acid metabolism, as well as derivatives and compounds related thereto, by alteration of the organism by inserting a non-natural pathway to intercept fatty acyl-ACP intermediates. In one nonlimiting embodiment, a thioesterase or a polypeptide having a thioesterase activity is introduced to generate free fatty acids. In one nonlimiting embodiment, a fatty acyl-CoA reductase is introduced to generate fatty alcohols. In one nonlimiting embodiment, an acyl-ACP reductase and/or aldehyde decarbonylase is introduced to generate alka(e)nes. Organisms produced in accordance with the present invention are useful in methods for biosynthesizing higher levels of compounds involved in fatty acid metabolism, derivatives thereof, and compounds related thereto.

For purposes of the present invention, “compounds involved in fatty acid metabolism” encompass fatty acids, fatty alcohols and alkane/alkenes as well as monofunctional, difunctional, branched chain or unsaturated C6-C20 products.

For purposes of the present invention, “derivatives and compounds related thereto” encompass compounds derived from the same substrates and/or enzymatic reactions as compounds involved in fatty acid metabolism, byproducts of these enzymatic reactions and compounds with similar chemical structure including, but not limited to, structural analogs wherein one or more substituents of compounds involved in serine metabolism are replaced with alternative substituents. Examples of related compounds which could be produced include, but are in no way limited to other monofunctional, difunctional, branched chain or unsaturated C6-C20 products.

For purposes of the present invention, “higher levels of compounds involved in fatty acid metabolism” means that the altered organisms and methods of the present invention are capable of producing increased levels of compounds involved in fatty acid metabolism and derivatives and compounds related thereto as compared to the same organism without alteration. In one nonlimiting embodiment, levels are increased by 2-fold or higher.

For compounds containing carboxylic acid groups such as organic monoacids, hydroxyacids, aminoacids and dicarboxylic acids, these compounds may be formed or converted to their ionic salt form when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate and/or bicarbonate, sodium hydroxide, ammonia and the like. The salt can be isolated as is from the system as the salt or converted to the free acid by reducing the pH to, for example, below the lowest pKa through addition of acid or treatment with an acidic ion exchange resin.

For compounds containing amine groups such as, but not limited to, organic amines, amino acids and diamine, these compounds may be formed or converted to their ionic salt form by addition of an acidic proton to the amine to form the ammonium salt, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid or muconic acid, and the like. The salt can be isolated as is from the system as a salt or converted to the free amine by raising the pH to, for example, above the highest pKa through addition of base or treatment with a basic ion exchange resin. Acceptable inorganic bases are known in the art and include aluminum hydroxide, calcium hydroxide, potassium hydroxide, sodium carbonate or bicarbonate, sodium hydroxide, and the like.

For compounds containing both amine groups and carboxylic acid groups such as, but not limited to, amino acids, these compounds may be formed or converted to their ionic salt form by either 1) acid addition salts, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include aluminum hydroxide, calcium hydroxide, potassium hydroxide, sodium carbonate and/or bicarbonate, sodium hydroxide, and the like, or 2) when an acidic proton present in the parent compound either is replaced by a metal ion, e.g., an alkali metal ion, an alkaline earth ion, or an aluminum ion; or coordinates with an organic base. Acceptable organic bases are known in the art and include ethanolamine, diethanolamine, triethanolamine, trimethylamine, N-methylglucamine, and the like. Acceptable inorganic bases are known in the art and include aluminum hydroxide, calcium hydroxide, potassium hydroxide, sodium carbonate, sodium hydroxide, ammonia and the like. The salt can be isolated as is from the system or converted to the free acid by reducing the pH to, for example, below the pKa through addition of acid or treatment with an acidic ion exchange resin. In one or more aspects of the invention, it is understood that the amino acid salt can be isolated as: i. at low pH, as the ammonium (salt)-free acid form; ii. at high pH, as the amine-carboxylic acid salt form; and/or iii. at neutral or midrange pH, as the free-amine acid form or zwitterion form.

In the process for biosynthesis of compounds involved in fatty acid metabolism and derivatives and compounds related thereto of the present invention, an organism capable of producing compounds involved in fatty acid metabolism and derivatives and compounds related thereto is obtained. The organism is then altered to produce more compounds involved in fatty acid metabolism and derivatives and compounds related thereto in the altered organism as compared to the unaltered organism.

In one nonlimiting embodiment, the organism is Cupriavidus necator (C. necator) or an organism with properties similar thereto. A nonlimiting embodiment of the organism is set for at lgcstandards-atcc with the extension .org/products/a11/17699.aspx?geo_country=gb#generalinformation of the world wide web.

C. necator (previously called Hydrogenomonas eutrophus, Alcaligenes eutropha, Raistonia eutropha, and Wautersia eutropha) is a Gram-negative, flagellated soil bacterium of the Betaproteobacteria class. This hydrogen-oxidizing bacterium is capable of growing at the interface of anaerobic and aerobic environments and easily adapts between heterotrophic and autotrophic lifestyles. Sources of energy for the bacterium include both organic compounds and hydrogen. Additional properties of C. necator include microaerophilicity, copper resistance (Makar, N. S. & Casida, L. E. Int. J. of Systematic Bacteriology 1987 37(4): 323-326), bacterial predation (Byrd et al. Can J Microbiol 1985 31:1157-1163; Sillman, C. E. & Casida, L. E. Can J Microbiol 1986 32:760-762; Zeph, L. E. & Casida, L. E. Applied and Environmental Microbiology 1986 52(4):819-823) and polyhydroxybutyrate (PHB) synthesis. In addition, the cells have been reported to be capable of both aerobic and nitrate dependent anaerobic growth. A nonlimiting example of a C. necator organism useful in the present invention is a C. necator of the H16 strain. In one nonlimiting embodiment, a C. necator host of the H16 strain with at least a portion of the phaCAB gene locus knocked out (ΔphaCAB) is used.

In another nonlimiting embodiment, the organism altered in the process of the present invention has one or more of the above-mentioned properties of Cupriavidus necator.

In another nonlimiting embodiment, the organism is selected from members of the genera Ralstonia, Wautersia, Cupriavidus, Alcaligenes, Burkholderia or Pandoraea.

For the process of the present invention, the organism is altered by inserting a non-natural pathway to intercept fatty acyl-ACP intermediates. In one nonlimiting embodiment, a thioesterase is inserted to generate free fatty acids. In one nonlimiting embodiment, a fatty acyl-CoA reductase is inserted to generate fatty alcohols. In one nonlimiting embodiment, an acyl-ACP reductase and/or aldehyde decarbonylase is inserted to generate alka(e)nes. In one nonlimiting embodiment an oxidoreductase and an acyl-ACP reductase is inserted to generate fatty alcohols. In one nonlimiting embodiment an acyl-CoA synthetase and a fatty acyl-CoA reductase is inserted to generate fatty alcohols. In one nonlimiting embodiment a thioesterase, an acyl-CoA synthetase and a fatty acyl-CoA reductase is inserted to generate fatty alcohols.

Exemplary organisms from which the thioesterase is derived include, but are not limited to, Weissella confusa, Clostridium argentinense, Lactococcus raffinolactis, Petunia integrifolia, Peptoniphilus harei, Clostridium botulinum, Spirochaeta smaragdinae, Eubacterium limosum, Escherichia coli, Lactococcus lactis, Clostridium sp., Haemophilus influenzae, Weissella paramesenteroides, Clostridiales bacterium, Streptococcus mitis, Bacteroides finegoldii, Solanum lycopersicum, Picea sitchensis, Pseudoramibacter alactolyticus, Bos Taurus, Alkaliphilus oremlandii, Desulfotomaculum nigrificans, Ceilulosilyticum lentocellum, Paenibacillus sp., Carboxydothermus hydrogenoformans, Clostridium carboxidivorans, Thermovirga lienii, Selaginella moellendorffii and Treponema caldarium.

In one nonlimiting embodiment, the thioesterase comprises E. coli ′tesA (SEQ ID NO:19), a truncated version of the full tesA lacking the N-terminal signal peptide, a thioesterase selected from SEQ ID NO: 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 or 81 or a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to an amino acid sequence set forth in SEQ ID NO: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 or 81 or a functional fragment thereof. In one nonlimiting embodiment, the thioesterase is encoded by a nucleic acid sequence comprising E. coli ′tesA (SEQ ID NO:20), a nucleic acid sequence selected from SEQ ID NO: 22, 24, 26, 28, 30, 32, 34, 36, 38, 40, 42, 44, 46, 48, 50, 52, 54, 56, 58, 60, 62, 64, 66, 68, 70, 72, 74, 76, 80 or 82 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 20, 22, 24, 26, 28, 30, 32, 34, 36, 38, 40, 42, 44, 46, 48, 50, 52, 54, 56, 58, 60, 62, 64, 66, 68, 70, 72, 74, 76, 80 or 82 or a functional fragment thereof.

In one nonlimiting embodiment, the fatty acyl-CoA reductase is from Bermanella marisrubri or Marinobacter algicola and comprises SEQ ID NO: 9 or 11 or a polypeptide with similar enzymatic activities exhibiting at least about 50%, 60%, 70%, 75%, 80%, 85%, 90%, 910, 92%, 930, 94%, 95%, 96%, 97%, 98%, 99% or 99.5% sequence identity to an amino acid sequence set forth in SEQ ID NO: 9 or 11 or a functional fragment thereof. In one nonlimiting embodiment, the fatty acyl-CoA reductase is from Bermanella marisrubri or Marinobacter algicola and is encoded by a nucleic acid sequence comprising SEQ ID NO: 10 or 12 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 10 or 12 or a functional fragment thereof.

In one nonlimiting embodiment, the acyl-ACP reductase is from Synechococcus and comprises SEQ ID NO:1 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 1 or a functional fragment thereof. In one nonlimiting embodiment, the acyl-ACP reductase is from Synechococcus and is encoded by a nucleic acid sequence comprising SEQ ID NO:2 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or a functional fragment thereof.

In one nonlimiting embodiment, the aldehyde decarbonylase is from Synechococcus and comprises SEQ ID NO:3 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 3 or a functional fragment thereof. In one nonlimiting embodiment, the aldehyde decarbonylase is from Synechococcus and is encoded by a nucleic acid sequence comprising SEQ ID NO:4 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 4 or a functional fragment thereof.

In one nonlimiting embodiment, the oxidoreductase is from E. coli and comprises SEQ ID NO:5 or a polypeptide with similar enzymatic activities exhibiting at least about 50%, 60%, 70%, 75%, 80%, 85%, 90%, 91%, 92%, 93%, 94%, 95%, 960, 97%, 98%, 99% or 99.5% sequence identity to an amino acid sequence set forth in SEQ ID NO: 5 or a functional fragment thereof. In one nonlimiting embodiment, the oxidoreductase is from E. coli and is encoded by a nucleic acid sequence comprising SEQ ID NO:6 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 6 or a functional fragment thereof.

In one nonlimiting embodiment, the acyl-CoA synthetase is from E. coli and comprises SEQ ID NO:7 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 7 or a functional fragment thereof. In one nonlimiting embodiment, the oxidoreductase is from E. coli and is encoded by a nucleic acid sequence comprising SEQ ID NO:8 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 8 or a functional fragment thereof.

In one nonlimiting embodiment, the nucleic acid sequence is codon optimized for C. necator.

In one nonlimiting embodiment, the organism is further altered to delete one or more enzymes of the β-oxidation pathway.

In one nonlimiting embodiment, the fatty acid is pimelic acid and the organism is further altered to delete one or more enzymes which activate pimelate. For example, one or more genes selected from A3350-51 (acyl-CoA ligase and transport genes), A1519-20 (acyl-CoA ligase and transport genes), and B1446-9 (acyl-CoA transferase, transport and regulatory gene) can be deleted. In one nonlimiting embodiment, the fatty acid is pimelic acid and the organism is further altered to inhibit acyl-CoA dehydrogenase. For example, one or more genes selected from A2818 (glutaryl-CoA dehydrogenase gene), B2555 (acyl-CoA dehydrogenase gene) and A0814-16 (electron transfer and acyl-CoA dehydrogenase genes) can be deleted. In one nonlimiting embodiment, the fatty acid is pimelic acid and the organism is further altered to delete a cluster selected from A0459-0464 (β-oxidation cluster 1) and A1526-1531 β-oxidation cluster 2).

In one nonlimiting embodiment, the fatty acid is adipic acid and the organism is further altered by deleting an adipic acid specific operon. In one nonlimiting embodiment, the adipic acid specific operon is B0198-202 (acyl-CoA transferase, thiolase, dehydrogenase and transport). In one nonlimiting embodiment, the fatty acid is adipic acid and the organism is further altered to delete one or more enzymes which activate adipate. For example, B1446-9 (acyl-CoA transferase, transport and regulatory gene) can be deleted. In one nonlimiting embodiment, the fatty acid is adipic acid and the organism is further altered to inhibit acyl-CoA dehydrogenase. For example, one or more genes selected from B2555 (acyl-CoA dehydrogenase gene), A1526-1531 (β-oxidation cluster 2), A2818 (glutaryl-CoA dehydrogenase gene), A0814-16 (electron transfer and acyl-CoA dehydrogenase genes) or A1067/68 (acyl-CoA dehydrogenase genes) can be deleted. In one nonlimiting embodiment, the fatty acid is adipic acid and the organism is further altered to delete A0459-0464 (β-oxidation cluster 1).

In one nonlimiting embodiment, the organism is further modified to eliminate phaCAB, involved in PHBs production and/or H16-A0006-9 encoding endonucleases thereby improving transformation efficiency as described in U.S. patent application Ser. No. 15/717,216, teachings of which are incorporated herein by reference.

In the process of the present invention, the altered organism is then subjected to conditions wherein compounds involved in fatty acid metabolism and derivatives and compounds related thereto are produced.

In the process described herein, a fermentation strategy can be used that entails anaerobic, micro-aerobic or aerobic cultivation. A fermentation strategy can entail nutrient limitation such as nitrogen, phosphate or oxygen limitation.

Under conditions of nutrient limitation, a phenomenon known as overflow metabolism (also known as energy spilling, uncoupling or spillage) occurs in many bacteria (Russell, 2007). In growth conditions in which there is a relative excess of carbon source and other nutrients (e.g. phosphorous, nitrogen and/or oxygen) are limiting cell growth, overflow metabolism results in the use of this excess energy (or carbon), not for biomass formation but for the excretion of metabolites, typically organic acids. In Cupriavidus necator a modified form of overflow metabolism occurs in which excess carbon is sunk intracellularly into the storage carbohydrate polyhydroxybutyrate (PHB). In strains of C. necator which are deficient in PHB synthesis this overflow metabolism can result in the production of extracellular overflow metabolites. The range of metabolites that have been detected in PHB deficient C. necator strains include acetate, acetone, butanoate, cis-aconitate, citrate, ethanol, fumarate, 3-hydroxybutanoate, propan-2-ol, malate, methanol, 2-methyl-propanoate, 2-methyl-butanoate, 3-methyl-butanoate, 2-oxoglutarate, meso-2,3-butanediol, acetoin, DL-2,3-butanediol, 2-methylpropan-1-ol, propan-1-ol, lactate 2-oxo-3-methylbutanoate, 2-oxo-3-methylpentanoate, propanoate, succinate, formic acid and pyruvate. The range of overflow metabolites produced in a particular fermentation can depend upon the limitation applied (e.g. nitrogen, phosphate, oxygen), the extent of the limitation, and the carbon source provided (Schlegel, H. G. & Vollbrecht, D. Journal of General Microbiology 1980 117:475-481; Steinbüchel, A. & Schlegel, H. G. Appl Microbiol Biotechnol 1989 31: 168; Vollbrecht et al. Eur J Appl Microbiol Biotechnol 1978 6:145-155; Vollbrecht et al. European J. Appl. Microbiol. Biotechnol. 1979 7: 267; Vollbrecht, D. & Schlegel, H. G. European J. Appl. Microbiol. Biotechnol. 1978 6: 157; Vollbrecht, D. & Schlegel, H. G. European J. Appl. Microbiol. Biotechnol. 1979 7: 259).

Applying a suitable nutrient limitation in defined fermentation conditions can thus result in an increase in the flux through a particular metabolic node. The application of this knowledge to C. necator strains genetically modified to produce desired chemical products via the same metabolic node can result in increased production of the desired product.

A cell retention strategy using a ceramic hollow fiber membrane can be employed to achieve and maintain a high cell density during fermentation. The principal carbon source fed to the fermentation can derive from a biological or non-biological feedstock. The biological feedstock can be, or can derive from, monosaccharides, disaccharides, lignocellulose, hemicellulose, cellulose, paper-pulp waste, black liquor, lignin, levulinic acid and formic acid, triglycerides, glycerol, fatty acids, agricultural waste, thin stillage, condensed distillers' solubles or municipal waste such as fruit peel/pulp. The non-biological feedstock can be, or can derive from, natural gas, syngas, CO₂/H₂, CO, H₂, O₂, methanol, ethanol, non-volatile residue (NVR) a caustic wash waste stream from cyclohexane oxidation processes or waste stream from a chemical industry such as, but not limited to a carbon black industry or a hydrogen-refining industry, or petrochemical industry, a nonlimiting example being a PTA-waste stream.

In one nonlimiting embodiment, at least one of the enzymatic conversions of the production method comprises gas fermentation within the altered Cupriavidus necator host, or a member of the genera Ralstonia, Wautersia, Alcaligenes, Burkholderia and Pandoraea, and other organism having one or more of the above-mentioned properties of Cupriavidus necator. In this embodiment, the gas fermentation may comprise at least one of natural gas, syngas, CO₂/H₂, CO, H₂, O₂, methanol, ethanol, non-volatile residue, caustic wash from cyclohexane oxidation processes, or waste stream from a chemical industry such as, but not limited to a carbon black industry or a hydrogen-refining industry, or petrochemical industry. In one nonlimiting embodiment, the gas fermentation comprises CO₂/H₂.

The methods of the present invention may further comprise recovering produced compounds involved in fatty acid metabolism or derivatives or compounds related thereto. Once produced, any method can be used to isolate the compound or compounds involved in fatty acid metabolism or derivatives or compounds related thereto.

The present invention also provides altered organisms capable of biosynthesizing increased amounts of compounds involved in fatty acid metabolism and derivatives and compounds related thereto as compared to the unaltered organism. In one nonlimiting embodiment, the altered organism of the present invention is a genetically engineered strain of Cupriavidus necator capable of producing compounds involved in fatty acid metabolism and derivatives and compounds related thereto. In another nonlimiting embodiment, the organism to be altered is selected from members of the genera Ralstonia, Wautersia, Alcaligenes, Cupriavidus, Burkholderia and Pandoraea, and other organisms having one or more of the above-mentioned properties of Cupriavidus necator. In one nonlimiting embodiment, the present invention relates to a substantially pure culture of the altered organism capable of producing compounds involved in fatty acid metabolism and derivatives and compounds related thereto comprising a non-natural pathway inserted to intercept fatty acyl-ACP intermediates. In one nonlimiting embodiment, a thioesterase is inserted to generate free fatty acids. In one nonlimiting embodiment, a fatty acyl-CoA reductase is inserted to generate fatty alcohols. In one nonlimiting embodiment, an acyl-ACP reductase and/or aldehyde decarbonylase is inserted to generate alka(e)nes.

As used herein, a “substantially pure culture” of an altered organism is a culture of that microorganism in which less than about 40% (i.e., less than about 35%; 30%; 25%; 20%; 15%; 10%; 5%; 2%; 1%; 0.50; 0.25%; 0.10; 0.010; 0.001%; 0.0001%; or even less) of the total number of viable cells in the culture are viable cells other than the altered microorganism, e.g., bacterial, fungal (including yeast), mycoplasmal, or protozoan cells. The term “about” in this context means that the relevant percentage can be 15% of the specified percentage above or below the specified percentage. Thus, for example, about 20% can be 17% to 23%. Such a culture of altered microorganisms includes the cells and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

Altered organisms of the present invention comprise an introduction of at least one synthetic gene encoding one or multiple enzyme(s).

In one nonlimiting embodiment, the altered organisms of the present invention may comprise at least one genome-integrated synthetic operon encoding an enzyme.

In one nonlimiting embodiment, the altered organism is produced by integration of a synthetic operon for a non-natural pathway to intercept fatty acyl-ACP intermediates. In one nonlimiting embodiment, the non-natural pathway comprises a thioesterase to generate free fatty acids. In one nonlimiting embodiment, the non-natural pathway comprises a fatty acyl-CoA reductase to generate fatty alcohols. In one nonlimiting embodiment, the non-natural pathway comprises an acyl-ACP reductase and/or aldehyde decarbonylase to generate alka(e)nes. In one nonlimiting embodiment an oxidoreductase and an acyl-ACP reductase is inserted to generate fatty alcohols. In one nonlimiting embodiment an acyl-CoA synthetase and a fatty acyl-CoA reductase is inserted to generate fatty alcohols. In one nonlimiting embodiment a thioesterase, an acyl-CoA synthetase and a fatty acyl-CoA reductase is inserted to generate fatty alcohols.

Exemplary organisms from which the thioesterase is derived include, but are not limited to, Weissella confusa, Clostridium argentinense, Lactococcus raffinolactis, Petunia integrifolia, Peptoniphilus harei, Clostridium botulinum, Spirochaeta smaragdinae, Eubacterium limosum, Escherichia coli, Lactococcus lactis, Clostridium sp., Haemophilus influenzae, Weissella paramesenteroides, Clostridiales bacterium, Streptococcus mitis, Bacteroides finegoldii, Solanum lycopersicum, Picea sitchensis, Pseudoramibacter alactolyticus, Bos Taurus, Alkaliphilus oremlandii, Desulfotomaculum nigrificans, Ceilulosilyticum lentocellum, Paenibacillus sp., Carboxydothermus hydrogenoformans, Clostridium carboxidivorans, Thermovirga lienii, Selaginella moellendorffii and Treponema caldarium.

In one nonlimiting embodiment, the thioesterase comprises E. coli ′tesA (SEQ ID NO:19), a truncated version of the full tesA lacking the N-terminal signal peptide, a thioesterase selected from SEQ ID NO: 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 or 81 or a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to an amino acid sequence set forth in SEQ ID NO: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 or 81 or a functional fragment thereof. In one nonlimiting embodiment, the thioesterase is encoded by a nucleic acid sequence comprising E. coli ′tesA (SEQ ID NO:20), a nucleic acid sequence selected from SEQ ID NO: 22, 24, 26, 28, 30, 32, 34, 36, 38, 40, 42, 44, 46, 48, 50, 52, 54, 56, 58, 60, 62, 64, 66, 68, 70, 72, 74, 76, 80 or 82 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 20, 22, 24, 26, 28, 30, 32, 34, 36, 38, 40, 42, 44, 46, 48, 50, 52, 54, 56, 58, 60, 62, 64, 66, 68, 70, 72, 74, 76, 80 or 82 or a functional fragment thereof.

In one nonlimiting embodiment, the fatty acyl-CoA reductase is from Bermanella marisrubri or Marinobacter algicola and comprises SEQ ID NO: 9 or 11 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 9 or 11 or a functional fragment thereof. In one nonlimiting embodiment, the fatty acyl-CoA reductase is from Bermanella marisrubri or Marinobacter algicola and is encoded by a nucleic acid sequence comprising SEQ ID NO: 10 or 12 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 10 or 12 or a functional fragment thereof.

In one nonlimiting embodiment, the acyl-ACP reductase is from Synechococcus and comprises SEQ ID NO:1 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 1 or a functional fragment thereof. In one nonlimiting embodiment, the acyl-ACP reductase is from Synechococcus and is encoded by a nucleic acid sequence comprising SEQ ID NO:2 or a nucleic acid sequence encoding a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO: 2 or a functional fragment thereof.

In one nonlimiting embodiment, the aldehyde decarbonylase is from Synechococcus and comprises SEQ ID NO:3 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 3 or a functional fragment thereof. In one nonlimiting embodiment, the aldehyde decarbonylase is from Synechococcus and is encoded by a nucleic acid sequence comprising SEQ ID NO:4 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 4 or a functional fragment thereof.

In one nonlimiting embodiment, the oxidoreductase is from E. coli and comprises SEQ ID NO:5 or a polypeptide with similar enzymatic activities exhibiting at least about 50%, 60%, 70%, 75%, 800, 85%, 90%, 91%, 92%, 93%, 94%, 95%, 96%, 97%, 98%, 99% or 99.5% sequence identity to an amino acid sequence set forth in SEQ ID NO: 5 or a functional fragment thereof. In one nonlimiting embodiment, the oxidoreductase is from E. coli and is encoded by a nucleic acid sequence comprising SEQ ID NO:6 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 6 or a functional fragment thereof.

In one nonlimiting embodiment, the acyl-CoA synthetase is from E. coli and comprises SEQ ID NO:7 or a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to an amino acid sequence set forth in SEQ ID NO: 7 or a functional fragment thereof. In one nonlimiting embodiment, the oxidoreductase is from E. coli and is encoded by a nucleic acid sequence comprising SEQ ID NO:8 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 8 or a functional fragment thereof.

In one nonlimiting embodiment, the nucleic acid sequence is codon optimized for C. necator.

In one nonlimiting embodiment, the organism is further altered to delete one or more enzymes of the β-oxidation pathway.

In one nonlimiting embodiment, the fatty acid is pimelic acid and the organism is further altered to delete one or more enzymes which activate pimelate. For example, one or more genes selected from A3350-51 (acyl-CoA ligase and transport genes), A1519-20 (acyl-CoA ligase and transport genes), and B1446-9 (acyl-CoA transferase, transport and regulatory gene) can be deleted. In one nonlimiting embodiment, the fatty acid is pimelic acid and the organism is further altered to inhibit acyl-CoA dehydrogenase. For example, one or more genes selected from A2818 (glutaryl-CoA dehydrogenase gene), B2555 (acyl-CoA dehydrogenase gene) and A0814-16 (electron transfer and acyl-CoA dehydrogenase genes) can be deleted. In one nonlimiting embodiment, the fatty acid is pimelic acid and the organism is further altered to delete a cluster selected from A0459-0464 (β-oxidation cluster 1) and A1526-1531 (β-oxidation cluster 2).

In one nonlimiting embodiment, the fatty acid is adipic acid and the organism is further altered by deleting an adipic acid specific operon. In one nonlimiting embodiment, the adipic acid specific operon is B0198-202 (acyl-CoA transferase, thiolase, dehydrogenase and transport). In one nonlimiting embodiment, the fatty acid is adipic acid and the organism is further altered to delete one or more enzymes which activate adipate. For example, B1446-9 (acyl-CoA transferase, transport and regulatory gene) can be deleted. In one nonlimiting embodiment, the fatty acid is adipic acid and the organism is further altered to inhibit acyl-CoA dehydrogenase. For example, one or more genes selected from B2555 (acyl-CoA dehydrogenase gene), A1526-1531 (β-oxidation cluster 2), A2818 (glutaryl-CoA dehydrogenase gene), A0814-16 (electron transfer and acyl-CoA dehydrogenase genes) or A1067/68 (acyl-CoA dehydrogenase genes) can be deleted. In one nonlimiting embodiment, the fatty acid is adipic acid and the organism is further altered to delete A0459-0464 (β-oxidation cluster 1).

In one nonlimiting embodiment, the organism is further modified to eliminate phaCAB, involved in PHBs production and/or H16-A0006-9 encoding endonucleases thereby improving transformation efficiency.

The percent identity (and/or homology) between two amino acid sequences as disclosed herein can be determined as follows. First, the amino acid sequences are aligned using the BLAST 2 Sequences (B12seq) program from the stand-alone version of BLAST containing BLASTP version 2.0.14. This stand-alone version of BLAST can be obtained from the U.S. government's National Center for Biotechnology Information web site (www with the extension ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the two compared sequences share homology (identity), then the designated output file will present those regions of homology as aligned sequences. If the two compared sequences do not share homology (identity), then the designated output file will not present aligned sequences. Similar procedures can be followed for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 90.11, 90.12, 90.13, and 90.14 is rounded down to 90.1, while 90.15, 90.16, 90.17, 90.18, and 90.19 is rounded up to 90.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the polypeptides or nucleic acid sequences described herein can also be used in the methods and organisms disclosed herein. The term “functional fragment” as used herein refers to a peptide fragment of a polypeptide or a nucleic acid sequence fragment encoding a peptide fragment of a polypeptide that has at least 25% (e.g., at least: 30%; 40%; 50%; 60%; 70%; 75%; 80%; 85%; 90%; 95%; 98%; 99%; 100%; or even greater than 100%) of the activity of the corresponding mature, full-length, polypeptide. The functional fragment can generally, but not always, be comprised of a continuous region of the polypeptide, wherein the region has functional activity.

Functional fragments may range in length from about 10% up to 99% (inclusive of all percentages in between) of the original full-length sequence.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagluttanin (HA), glutathione-S-transferase (GST), or maltose binding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Endogenous genes of the organisms altered for use in the present invention also can be disrupted to prevent the formation of undesirable metabolites or prevent the loss of intermediates through other enzymes acting on such intermediates. In one nonlimiting embodiment, the organism is further altered to delete one or more enzymes of the β-oxidation pathway. In one nonlimiting embodiment, the organism is further modified to eliminate phaCAB, involved in PHBs production and/or H16-A0006-9 encoding endonucleases thereby improving transformation efficiency.

Thus, as described herein, altered organisms can include exogenous nucleic acids for non-natural pathways to intercept fatty acyl-ACP intermediates. In one nonlimiting embodiment, the exogenous nucleic acid encodes a thioesterase to generate free fatty acids. In one nonlimiting embodiment, the exogenous nucleic acid encodes a fatty acyl-CoA reductase to generate fatty alcohols. In one nonlimiting embodiment, the exogenous nucleic acid encodes an acyl-ACP reductase and/or aldehyde decarbonylase to generate alka(e)nes.

The term “exogenous” as used herein with reference to a nucleic acid (or a protein) and an organism refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host or organism once in or utilized by the host or organism. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

The present invention also provides exogenous genetic molecules of the nonnaturally occurring organisms disclosed herein such as, but not limited to, codon optimized nucleic acid sequences, expression constructs and/or synthetic operons.

In one nonlimiting embodiment, the exogenous genetic molecule comprises a codon optimized nucleic acid sequence encoding an enzyme of a non-natural pathway to intercept fatty acyl-ACP intermediates as disclosed herein. In one nonlimiting embodiment, the exogenous genetic molecule comprises a codon optimized nucleic acid sequence encoding a thioesterase, as disclosed herein, to generate free fatty acids. In one nonlimiting embodiment, the exogenous genetic molecule comprises a codon optimized nucleic acid sequence encoding a fatty acyl-CoA reductase, as disclosed herein, to generate fatty alcohols. In one nonlimiting embodiment, the exogenous genetic molecule comprises a codon optimized nucleic acid sequence encoding a thioesterase, acyl-ACP reductase and/or aldehyde decarbonylase and/or oxidoreductase and/or acyl CoA synthetase, as disclosed herein. In one nonlimiting embodiment, the nucleic acid sequence is codon optimized for C. necator. Additional nonlimiting examples of exogenous genetic molecules include expression constructs and synthetic operons encoding one or more enzymes of a non-natural pathway to intercept fatty acyl-ACP intermediates. In one nonlimiting embodiment, the expression construct or synthetic operon is for a thioesterase, a fatty acyl-CoA reductase, an aldehyde decarbonylase, an oxidoreductase and/or an acyl-CoA synthetase as disclosed herein.

Also provided by the present invention are compounds involved in fatty acid metabolism and derivatives and compounds related thereto bioderived from an altered organism according to any of methods described herein.

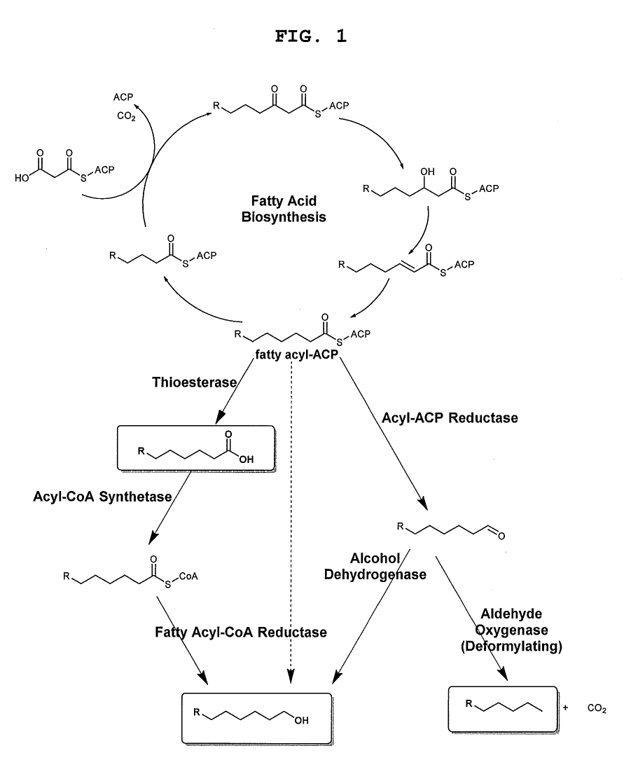

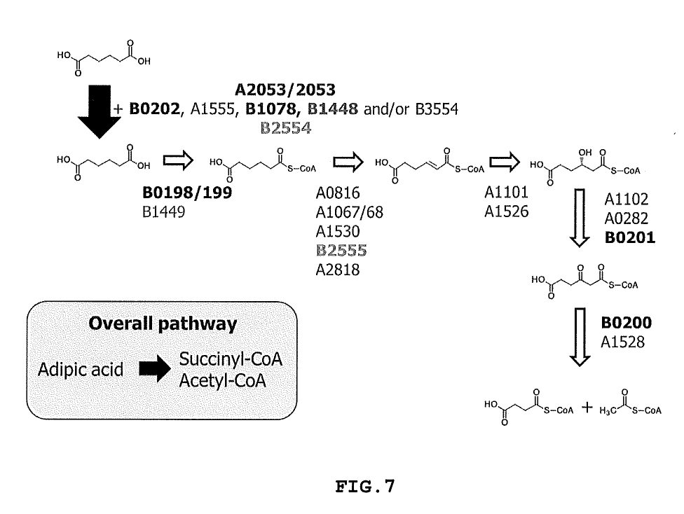

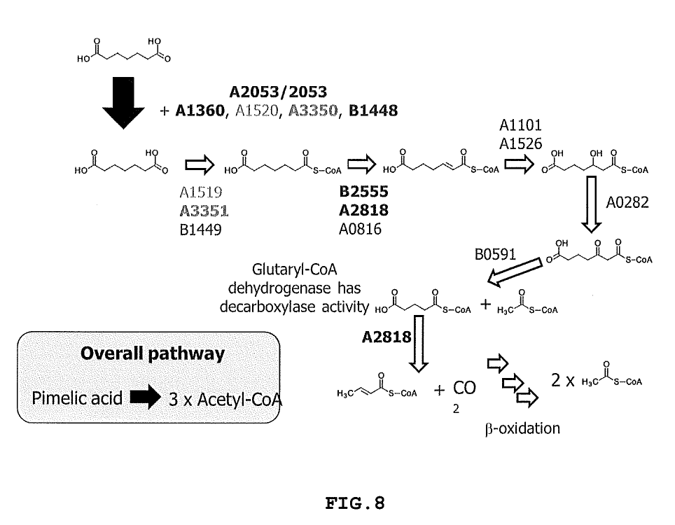

Further, the present invention relates to means and processes for use of these means for biosynthesis of compounds involved in fatty acid metabolism, and/or derivatives thereof and/or other compounds related thereto. Nonlimiting examples of such means include altered organisms and exogenous genetic molecules as described herein as well as any of the molecules as depicted in FIGS. 1, 7 and 8.

In addition, the present invention provides bio-derived, bio-based, or fermentation-derived products produced using the methods and/or altered organisms disclosed herein. In one nonlimiting embodiment, a bio-derived, bio-based or fermentation derived product is produced in accordance with the exemplary central metabolism depicted in FIG. 1, 7 or 8. Examples of such products include, but are not limited to, compositions comprising at least one bio-derived, bio-based, or fermentation-derived compound or any combination thereof, as well as molded substances, formulations and semi-solid or non-semi-solid streams comprising one or more of the bio-derived, bio-based, or fermentation-derived compounds or compositions, combinations or products thereof.

In one aspect of the present invention, metabolic flux through the C. necator fatty acid biosynthesis pathway was investigated by inserting non-natural pathways to intercept fatty acyl-ACP intermediates. Three different pathways were introduced to intercept the fatty acid pathway; thioesterases to generate free fatty acids; fatty acyl-CoA reductase to generate fatty alcohols, and; acyl-ACP reductase/aldehyde decarbonylase to generate alka(e)nes.

In one aspect of the present invention, two strain backgrounds were used, a strain lacking the PHA biosynthesis genes (AphaCAB) and a strain which in addition had deletions in β-oxidation pathways. Strains were investigated in both shake flask and in the Ambr15f small scale fermentation system.

In one aspect of the present invention, the engineered or biosynthetic pathways were found to function in shake-flask assays, with fatty acids, fatty alcohols and alkanes detected. The major fatty acids detected were palmitoleic, oleic and palmitic acids, the major fatty alcohol detected was hexadecanol and the major alkane detected was pentadecane. In one aspect of the present invention, additional putative products derived from fatty acids were also detected (e.g. aldehydes and ketones). Data from Ambr15f fermentation runs gave data showing maximum titers of ˜70 ppm for fatty acids, ˜45 ppm for alkanes and <1 ppm for fatty alcohols. Higher titers for fatty acids (˜200 ppm) were obtained in a strain that also co-expressed a heterologous ACC pathway.

In one aspect of the present invention, C. necator strains 001, 002, 003, 004, 005, 006, 007, 008, 009 and 010 (Table 3) were assessed for their ability to grow on C7, C10 and C18 fatty acids as sole carbon sources in comparison to fructose. While all strains were able to grow on fructose, there were some differences observed with the fatty acid substrates. No growth was observed on heptanoic acid for any of the strains. In one aspect of the present invention, due to the insolubility of decanoic and oleic acids it was not possible to observe growth by following OD₆₀₀. In the cultures with oleic acid added, however, noticeable clearance of the culture media was observed in some of the cultures, showing apparent metabolism of oleic acid. No differences were observed in the decanoic acid incubated cultures.

In one aspect of the present invention, upon visual inspection of the oleic acid incubated cultures, strains were categorized into 3 groups (see Table 3 for genotypes):

No apparent metabolism of oleic acid: strains 005, 006, 008, 009, possible metabolism of oleic acid: strains 002, 003, 010, and clearer metabolism of oleic acid: strains 001, 007.

Three of the strains with the clearest non-metabolizing phenotype had the double β-oxidation deletion ΔA0459-464, ΔA1526-31 (see Table 3).

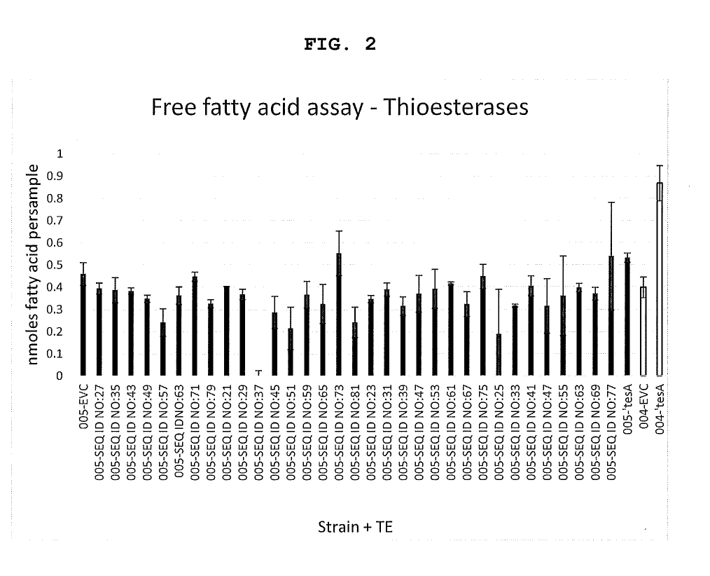

In one aspect of the present invention, plasmids for expression of thioesterases under the control of Pₗₐₜₑ were used to transform C. necator strains 004 (AphaCAB, ΔA0006-9) and 005 (ΔphaCAB, ΔB0356-0404, ΔA3350-3351, ΔB1446-9, ΔA1519-20, ΔA-9, ΔA0459-464, ΔA1526-31). These strains were then assessed for total fatty acid production as disclosed herein. A total of 34 TEs were assessed in the β-oxidation deficient strain 005 background and only one was assessed in the ΔphaCAB, ΔA0006-9 background (strain 004). FIG. 2 shows the results of the analysis of free fatty acids for these strains. Little difference in overall fatty acid content was observed between empty vector control strains and thioesterase expressing strains for in the β-oxidation deficient ΔphaCAB, ΔB0356-0404, ΔA3350-3351, ΔB1446-9, ΔA1519-20, ΔA0006-9, ΔA0459-464, ΔA1526-31 background (strain 005). However, in the ΔphaCAB, ΔA0006-9 background (strain 004), a clear increase in fatty acid content was observed upon expression of ′tesA.

In one aspect of the present invention, cultures for the production of fatty acid derived molecules were grown as disclosed herein for shake flask assessment.

Production of alkanes is via the interception of fatty acyl-ACP with acyl ACP-reductase and (AAR) aldehyde oxygenase (ADO) (Schirmer et al. Science. 2010 329(5991):559-62). Wild type and β-oxidation deficient C. necator hosts were transformed with plasmids encoding AAR and ADO genes (SEQ ID NO: 2 and SEQ ID NO: 4 and 0825) to give strains S2 and S11. This strategy has previously been used successfully for the production of fatty alkanes in C. necator H16 (Crepin et al. Metab Eng. 2016 September; 37:92-101). These strains together with empty vector controls and strains bearing partial pathways were assessed for their ability to produce alkane products in shake flask cultures with and without a dodecane layer. Alkane products were extracted from whole broth or pellets before analysis. In the case of cultures incubated with a dodecane layer the organic phase was used directly.

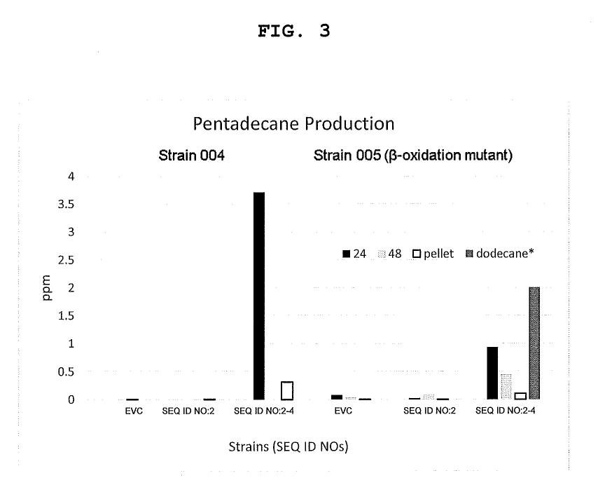

Data for pentadecane production is shown in FIG. 3. In one aspect of the present invention, alkanes were clearly detected in strains expressing AAR and ADO genes, with pentadecane being the major product. A product consistent with heptadecene was also observed and in all cases was estimated to be around ⅓^{rd}, the level of pentadecane produced. In broth samples the maximum level of total alka(e)ne observed was ˜4.8 ppm. This was observed in a non-β-oxidation mutant strain, the equivalent time point from the β-oxidation mutant background gave levels of ˜1.2 ppm. Analysis of cell pellets showed a similar pattern with around 3 fold more alkane product detected from the non-β-oxidation mutant strain.

In one aspect of the present invention, production of fatty alcohols is via reduction of fatty acyl CoA with fatty acyl CoA reductase (FAR). These enzymes have been disclosed to function with both fatty acyl-CoA and fatty acyl-ACP as substrates but the preferred substrates are the CoA thioesters. For production of fatty alcohols two variants of FAR enzymes were analyzed (SEQ ID NO: 10 from Marinobacter algicola DG893 and SEQ ID NO: 12 from Bermanella marisrubri). These were expressed with and without additional genes, SEQ ID NO: 8 (E. coli FadD to convert free fatty acids to CoA thioesters) and SEQ ID NO: 6 (E. coli oxidoreductase YbbO to reduce any aldehyde products to the respective alcohols). An additional strategy, expressing AAR gene (SEQ ID NO:84) together with oxidoreductase YbbO was also assessed for fatty alcohol production.

In one aspect of the present invention, these strains together with empty vector controls and strains bearing partial pathways were assessed for their ability to produce alcohol products in shake flask cultures. Alcohol products were extracted from whole broth or pellets and derivatized before analysis as described.

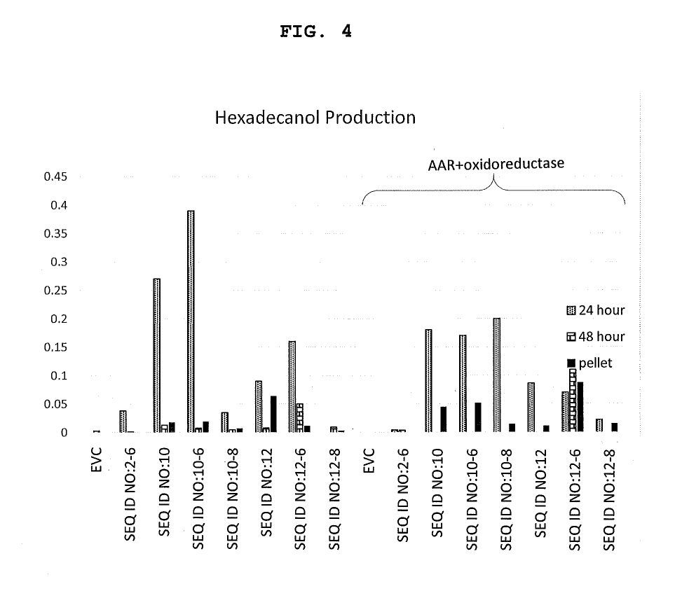

Data for fatty alcohol production is shown in FIG. 4. Fatty alcohols were clearly detected in strains expressing FAR genes while in strains expressing AAR plus oxidoreductase detected levels of alcohols were <0.05 ppm, similar to some of the negative controls. Levels of hexadecanol were below 0.4 ppm for all producing strains.

In one aspect of the present invention, the Ambr15f system was used to give similar and controlled growth conditions for all strains.

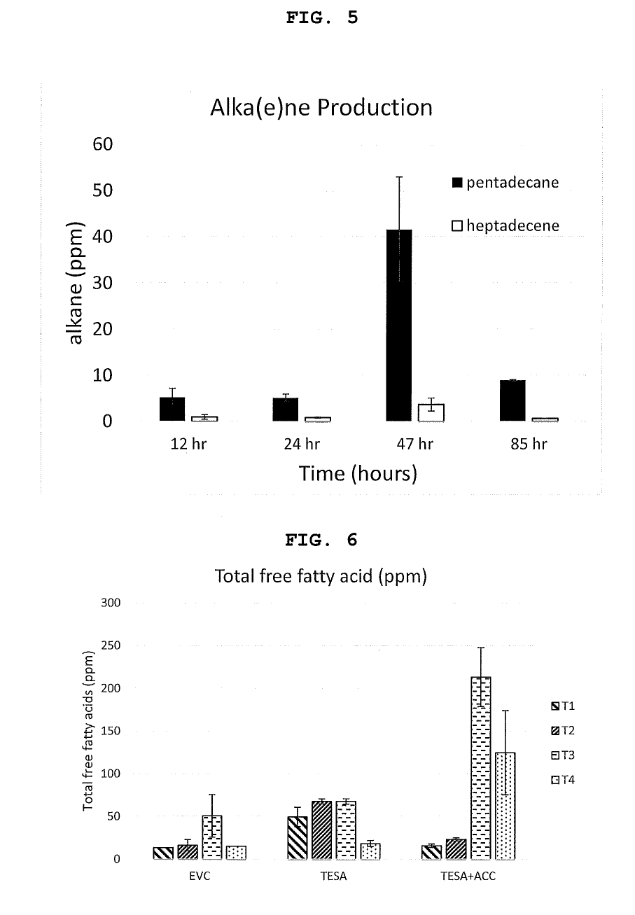

Strain S11, which expresses AAR and ADO in a β-oxidation mutant background was used to assess the production of alkanes in the Ambr15 system, together with a control strain bearing an empty vector. In one aspect of the present invention, 500 μL samples were taken at four time points and alkanes were extracted and analyzed as described. Data for alkane production (FIG. 5) shows that the highest levels of alkanes were detected at 47 hours, with levels of alkanes subsequently dropping when the feed was stopped, indicating the possible consumption of the alkane products. The major alkane detected was pentadecane with heptadecene being the other quantified product. No alkanes were detected in the control strain.

To assess the production of fatty alcohols from expression of the acyl-CoA reductase genes strains S15, S17, S18 and S19 (EVC) were cultured in the Ambr15f system. 500 uL samples were taken at four timepoints for extraction and analysis. In one aspect of the present invention, levels of fatty alcohols detected were below 1 ppm in all cases.

To assess the production of fatty acids from expression of the thioesterase ′tesA strains S21 (EVC), S22 (P_{Lac}-′tesA) and S23 (P_{araBAD}-dtsR1accBCE_{Cg}: P_{Lac}-′tesA) were cultured in the Ambr15f system. In one aspect of the present invention, cultures were supplemented with biotin (40 μg/L) which increased fatty acid titers in shake flasks. 500 μL samples were taken at four timepoints for fatty acid extraction and analysis. Total free fatty acid levels are shown in FIG. 6 (major fatty acids were palmitic, palmitoleic, stearic and an isomer of oleic acid). In one aspect of the present invention, expression of ′tesA alone resulted in an increase in free fatty acid titers at the earlier timepoints (T1 and T2). At the later time points, including the maximum titer point, the increases over the empty vector control (EVC) are less significant. Expression of ′tesA together with ACC, however, resulted in a significant increase in free fatty acid titers at the later time points and the maximum titers obtained of ˜200 ppm at T3. In one aspect of the present invention, at T4 free fatty acid titers drop in all cases indicating the consumption of fatty acids in these strains at this later time point.

In this experiment methylketones were also detected. These compounds are products of the incomplete β-oxidation of fatty acids and have previously been detected in C. necator (Muller et al. Appl Environ Microbiol. 2013 79(14):4433-9).

In one aspect of the present invention, the organism can be further altered to delete one or more enzymes of the β-oxidation pathway.

In one nonlimiting embodiment, the fatty acid is pimelic acid and the organism is further altered to delete one or more enzymes which activate pimelate. For example, one or more genes selected from A3350-51 (acyl-CoA ligase and transport genes), A1519-20 (acyl-CoA ligase and transport genes), and B1446-9 (acyl-CoA transferase, transport and regulatory gene) can be deleted. In one nonlimiting embodiment, the fatty acid is pimelic acid and the organism is further altered to inhibit acyl-CoA dehydrogenase. For example, one or more genes selected from A2818 (glutaryl-CoA dehydrogenase gene), B2555 (acyl-CoA dehydrogenase gene) and A0814-16 (electron transfer and acyl-CoA dehydrogenase genes) can be deleted. In one nonlimiting embodiment, the fatty acid is pimelic acid and the organism is further altered to delete a cluster selected from A0459-0464 (β-oxidation cluster 1) and A1526-1531 (β-oxidation cluster 2).

In one nonlimiting embodiment, the fatty acid is adipic acid and the organism is further altered by deleting an adipic acid specific operon. In one nonlimiting embodiment, the adipic acid specific operon is B0198-202 (acyl-CoA transferase, thiolase, dehydrogenase and transport). In one nonlimiting embodiment, the fatty acid is adipic acid and the organism is further altered to delete one or more enzymes which activate adipate. For example, B1446-9 (acyl-CoA transferase, transport and regulatory gene) can be deleted. In one nonlimiting embodiment, the fatty acid is adipic acid and the organism is further altered to inhibit acyl-CoA dehydrogenase. For example, one or more genes selected from B2555 (acyl-CoA dehydrogenase gene), A1526-1531 (β-oxidation cluster 2), A2818 (glutaryl-CoA dehydrogenase gene), A0814-16 (electron transfer and acyl-CoA dehydrogenase genes) or A1067/68 (acyl-CoA dehydrogenase genes) can be deleted. In one nonlimiting embodiment, the fatty acid is adipic acid and the organism is further altered to delete A0459-0464 (β-oxidation cluster 1).

Although specific advantages have been enumerated above, various embodiments may include some, none, or all of the enumerated advantages. Further, other technical advantages may become readily apparent to one of ordinary skill in the art after review of the figures and description herein. It should be understood at the outset that, although exemplary embodiments are described herein, the principles of the present disclosure may be implemented using any number of techniques, whether currently known or not. The present disclosure should in no way be limited to the exemplary implementations and techniques described herein.

Modifications, additions, or omissions may be made to the compositions, systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

The following section provides further illustration of the methods and materials of the present invention. These Examples are illustrative only and are not intended to limit the scope of the invention in any way.

### Examples

All plasmids were constructed using standard cloning techniques such as described, for example in Green and Sambrook, Molecular Cloning, A Laboratory Manual, Nov. 18, 2014.

Synthetic genes used are listed in Table 1.

Plasmids constructed are listed in Table 2.

C. necator strains used are listed in Tables 3 and 4. C. necator transformations were carried out using a standard electroporation protocol.

TABLE 1
DNA parts used in assembly of pathway constructs

| SEQ ID | | Accession | Anti- |
|---|---|---|---|
| NO: | Encoded activity | number | biotic |
| SEQ ID NO: 2 | Long-chain acyl- | WP_011242364.1 | Amp |
| | [acyl-carrier- | | |
| | protein] reductase | | |
| | [Synechococcus] | | |
| SEQ ID NO: 4 | Aldehyde oxygenase | WP_011378104.1 | Amp |
| | (deformylating) | | |
| | [Synechococcus] | | |
| SEQ ID NO: 6 | Oxidoreductase YbbO | NP_415026.1 | Amp |
| | [Escherichia coli | | |
| | K-12, MG1655] | | |
| SEQ ID NO: 8 | Fatty acyl-CoA | NP_416319.1 | Amp |
| | synthetase (FadD) | | |
| | [Escherichia coli | | |
| | K-12, MG1655] | | |
| SEQ ID NO: 10 | Fatty acyl-CoA | A6EVI7 | Amp |
| | reductase | | |
| | [Marinobacter | | |
| | algicola DG893] | | |
| SEQ ID NO: 12 | Fatty acyl-CoA | Q1N697 | Amp |
| | reductase | | |
| | (Bermanella | | |
| | marisrubri) | | |
| pBBR-1A-BAD* | Recipient vector | N/A | Kan |
| SEQ ID NO: 83 | rnpBT1 terminator | N/A | Amp |
| SEQ ID NO: 14 | C. glutamicum dtsR1 | NP_599940.1 | Amp |
| SEQ ID NO: 16 | C. glutamicum AccBC | NP_599932.1 | Amp |
| SEQ ID NO: 18 | C. glutamicum AccE | NP_599938.1 | Amp |
| SEQ ID NO: 20 | E. coli 'tesA | | |

*This 1A vector is a derivative of pBBR1-MCS2 (described at sciencedirect with the extension .com/science/article/pii/0378111995005841 of the world wide web) altered for compatibility with DNA assembly techniques described herein.

TABLE 2
Pathway constructs

| Plasmid name | Antibiotic | Parts |
|---|---|---|
| pBBR1-BAD-SEQ ID NO: 2 | Kan | P_{araBAD}-SEQ ID NO: 2- rnpBT1 |
| pBBR1-BAD-SEQ ID NO: | Kan | P_{araBAD}-SEQ ID NO: 2-SEQ ID |
| 2-SEQ ID NO: 4 | | NO: 4- rnpBT1 |
| pBBR1-BAD-SEQ ID NO: | Kan | P_{araBAD}-SEQ ID NO: 2- SEQ ID |
| 2-SEQ ID NO: 6 | | NO: 6 - rnpBT1 |
| pBBR1-BAD-SEQ ID NO: 10 | Kan | P_{araBAD}-SEQ ID NO: 10-rnpBT1 |
| pBBR1-BAD-SEQ ID NO: 12 | Kan | P_{araBAD} - SEQ ID NO: 12 - - |
| | | rnpBT1 |
| pBBR1-BAD-SEQ ID NO: | Kan | P_{araBAD}-SEQ ID NO: 10-SEQ ID |
| 10-SEQ ID NO: 6 | | NO: 6- rnpBT1 |
| pBBR1-BAD-SEQ ID NO: | Kan | P_{araBAD}-SEQ ID NO: 10-SEQ ID |
| 10-SEQ ID NO: 8 | | NO: 8- rnpBT1 |
| pBBR1-BAD-SEQ ID NO: | Kan | P_{araBAD}-SEQ ID NO: 12-SEQ ID |
| 12-SEQ ID NO: 6 | | NO: 6 - rnpBT1 |
| pBBR1-BAD-SEQ ID NO: | Kan | P_{araBAD}-SEQ ID NO: 12-SEQ ID |
| 12-SEQ ID NO: 8 | | NO: 8- rnpBT1 |
| Empty vector control | Kan | EVC |
| pBBR1-BAD-SEQ ID NO: 14- SEQ ID NO: | Tet | P_{araBAD}-SEQ ID NO: 14 - SEQ |
| 16- SEQ ID NO: 18 | | ID NO: 16-SEQ ID NO: 18- |
| | | rnpBT1: P_{lac}-SEQ IS NO: 20 |
| pBBR1-BAD-SEQ ID NO: 20 | Kan | P_{lac}-SEQ ID NO: 20 |

TABLE 3
C. necator host strains used

| Strain | Genotype |
|---|---|
| C. necator | ΔphaCAB |
| H16 | |
| C. necator | ΔphaCAB, ΔB0356-0404, ΔA3350-3351, ΔB1446-9, ΔA1519- |
| H16 | 20, ΔA0006-9, ΔA2770 (18) |
| C. necator | ΔphaCAB, ΔB0356-0404, ΔA3350-3351, ΔB1446-9, ΔA1519- |
| H16 | 20, ΔA0006-9, ΔA2770 (20) |
| C. necator | ΔphaCAB, ΔA0006-9 (clone 1) |
| H16 | |
| C. necator | ΔphaCAB, ΔB0356-0404, ΔA3350-3351, ΔB1446-9, ΔA1519- |
| H16 | 20, ΔA0006-9, ΔA0459-464, ΔA1526-31 (2) |
| C. necator | ΔphaCAB, ΔB0356-0404, ΔA3350-3351, ΔB1446-9, ΔA1519- |
| H16 | 20, ΔA0006-9, ΔA0459-464, ΔA1526-31 (15) |
| C. necator | ΔphaCAB, ΔB0356-0404, ΔA2817-18, ΔA0006-9, ΔB2554-5, |
| H16 | ΔA0816 (3-10) |
| C. necator | ΔphaCAB, ΔB0356-0404, ΔA2817-18, ΔA0006-9, ΔB2554-5, |
| H16 | ΔA0816 (2-18) |
| C. necator | ΔphaCAB, ΔB0356-0404, ΔA3350-3351, ΔB1446-9, ΔA1519- |
| H16 | 20, ΔA0006-9, ΔA0459-464, ΔA1526-31, ΔB0198-202, |
| | ΔA2817-18, ΔB2554-5, ΔA2770, ΔA0816 (4-4) |
| C. necator | ΔphaCAB, ΔB0356-0404, ΔA3350-3351, ΔB1446-9, ΔA1519- |
| H16 | 20, ΔA0006-9, ΔA0459-464, ΔA1526-31, ΔB0198-202, |
| | ΔA2817-18, ΔB2554-5, ΔA2770, ΔA0816 (22-3) |

TABLE 4
C. necator expression strains used

| Strain | Host | | |
|---|---|---|---|
| # | Strain | Plasmid | Antibiotic |
| S1 | 004 | pBBR1-BAD-SEQ ID | Kan |
| | | NO: 2 | |
| S2 | 004 | pBBR1-BAD-SEQ ID: 2- | Kan |
| | | SEQ ID NO: 4 | |
| S3 | 004 | pBBR1-BAD-SEQ ID | Kan |
| | | NO: 2-SEQ ID NO: 6 | |
| S4 | 004 | pBBR1-BAD-SEQ ID | Kan |
| | | NO: 10 | |
| S5 | 004 | pBBRl-BAD-SEQ ID | Kan |
| | | NO: 12 | |
| S6 | 004 | pBBR1-BAD-SEQ ID | Kan |
| | | NO: 10-SEQ ID NO: 6 | |
| S7 | 004 | pBBR1-BAD-SEQ ID | Kan |
| | | NO: 10-SEQ ID NO: 8 | |
| S8 | 004 | pBBR1-BAD-SEQ ID | Kan |
| | | NO: 12-SEQ ID NO: 6 | |
| S9 | 004 | pBBR1-BAD-SEQ ID | Kan |
| | | NO: 12-SEQ ID NO: 8 | |
| S10 | 005 | pBBR1-BAD-SEQ ID | Kan |
| | | NO: 2 | |
| S11 | 005 | pBBR1-BAD-SEQ ID | Kan |
| | | NO: 2-SEQ ID NO: 4 | |
| S12 | 005 | pBBR1-BAD-SEQ ID | Kan |
| | | NO: 2-SEQ ID NO: 6 | |
| S13 | 005 | pBBR1-BAD-SEQ ID | Kan |
| | | NO: 10 | |
| S14 | 005 | pBBR1-BAD-SEQ ID | Kan |
| | | NO: 12 | |
| S15 | 005 | pBBR1-BAD-SEQ ID | Kan |
| | | NO: 10-SEQ ID NO: 6 | |
| S16 | 005 | pBBR1-BAD-828-827 | Kan |
| S17 | 005 | pBBR1-BAD-SEQ ID | Kan |
| | | NO: 12-SEQ ID NO: 6 | |
| S18 | 005 | pBBR1-BAD-SEQ ID | Kan |
| | | NO: 12-SEQ ID NO: 8 | |
| S19 | 004 | pBBR1-BAD-1A | Kan |
| S20 | 005 | pBBR1-BAD-1A | Kan |
| S21 | 005 | pBBR1-2A-P_{araBAD} - | |
| | | BDIGENE933- | |
| | | BDIGENE935-rrnBT1- | |
| | | pLac-BDIGENE0640 | |
| S22 | 005 | pBBR-1B-pLac-TesA | |
| S23 | 005 | EVC | |

### Growth Conditions

For standard growth and maintenance C. necator strains were grown in Tryptic Soy Broth without Dextrose (TSB-G) broth and agar. For plasmid maintenance kanamycin was added at 300 mg/L.

For analysis of the ability of C. necator H16 and β-oxidation mutant strains to grow on fatty acids strains were grown overnight in 5 mL TSB-G broth (30° C., 220 rpm). Cultures were harvested by centrifugation then resuspended. The centrifugation step was repeated to wash the cells and these were inoculated into modified broth at a 1:40 dilution. The modified broth did not contain fructose but included alternative carbon sources at 5 g/L (fructose, heptanoic acid, decanoic acid or oleic acid). Cultures were incubated and monitored for turbidity indicative of growth.

For production of fatty acid derived products, strains were grown overnight in 5 mL TSB-G broth (30° C., 220 rpm). Cultures were harvested by centrifugation (3220×g, 10 minutes), then resuspended in a minimal medium adapted from Peoples and Sinskey (J Biol Chem 1989 264:15298-15303) and inoculated into minimal media. Cultures were incubated and after 6 hrs of growth L-arabinose was added to 0.3% to induce the P_{araBAD} promoter and where indicated dodecane was added at 0.1 volume of total culture.

Total unclarified broth samples, pellet samples, clarified broth samples and dodecane layer samples were collected for analyses.

### Ambr15

The Ambr15f is a small scale (15 ml), moderately high throughput (24 vessels) semi-automated fermentation platform. It encompasses many of the characteristics of a continuous stirrer tank reactor or CSTR such as temperature, pH and DO control, media feeding (exponential, linear, constant) as well as the ability to feed air, oxygen and nitrogen gases.

Strains from each pathway of the present invention, that demonstrated production at the flask/tube scale, were further screened in the Ambr15f under fed batch conditions with fructose as the sole carbon source. Several samples were taken over the course of the batch and feeding portions of growth, and target molecules accessed via GC or LCMS.

The screening methodology of the present invention allowed productivity to be quantified in high cell density cultures under stringent control, the potential for pathways to achieve high titers in a simple, scalable process.

### Seed Train

Cultures were first incubated overnight in the minimal media supplemented with appropriate antibiotic. Cultures were then sub-cultured to minimal media and further incubated for 16 hours. These were used as a direct inoculum for the fermentation fed batch cultures.

### Fermentation

The Sartorius Ambr15F platform was used to screen pathway strains in a fed batch mode of operation. This system allowed control of multiple variables such as dissolved oxygen and pH.

The following process conditions were standardized and run according to manufacturer's instructions.

Each vessel (total volume 15 ml) was loaded with 8 ml of batch growth media and manufacturer instructions were followed.

Cultures were then allowed to grow under defined conditions for the duration of the experiment. Samples (500 μl) were taken periodically with typically 4 over the course of the run to coincide with growth stages of induction (12 hours after inoculation), 12 hours post feed (24 hours after inoculation), end of feed (48 hours after inoculation) and end of run (72 hours).

### Analytical Methods

Enzymatic Analysis of Free Fatty Acids

The Free Fatty Acid Quantitation Kit (Sigma-Aldrich®-MAK044) was used for analysis of total free fatty acids in bacterial cultures.

Analysis of Fatty Acids and Fatty Alcohols and Instrumental GCMS Method Conditions

500 μl of sample (resuspended pellets or broth) was extracted with 500 μl of mixture chloroform:methanol (1:2) for one hour at 1400 rpm, 30° C. 500 μl of hexane was added and extracted for one hour, 1400 rpm, 30° C. The samples were centrifuged for 30 minutes at 1,500×g and 400 μl of the top layer was transferred to a vial and taken into dryness in the Genevac. 100 μl of MSTFA were added and incubated at 37° C. for 30 minutes and injected directly into the GCMS (1 μl).

For fatty alcohol analysis, a variation was also used, in which, following extraction and centrifugation a sample of the top layer (1 μL) was injected directly into the GCMS (1 μl) prior to derivatization. See Table 6 for GCMS conditions 2000 ppm stock solutions in acetone and/or hexane were used to prepare the substocks for the calibration curve. The following concentrations were used to generate standard curves: 1.25 ppm, 2.5 ppm, 5 ppm, 10 ppm, 20 ppm, 40 ppm.

TABLE 5
GCMS CONDITIONS

| PARAMETER | VALUE | |
|---|---|---|
| Carrier Gas | Helium at constant flow (1.0 ml/min) | |
| Injector | Split ratio | Splitless |
| | Temperature | 250° C. |
| Detector | Source Temperature | 230° C. |
| | Quad Temperature | 150° C. |
| | Interface | 260° C. |
| | Gain | 1 |
| | Scan Range | m/z 50-600 |
| | Threshold | 150 |
| | A/D samples* | 8 |
| | Scan Speed* | 781 (N = 3) |
| | Frequency (scans/sec)* | 1.5 |
| | Mode | SCAN |
| Solvent delay* | 5.0 min | |
| Oven Temperature | Initial T: 60° C. × 1.00 min | |
| Oven Ramp | 10° C./min to 325° C. for 10 min | |

| | |
|---|---|
| Injection volume | 1 μl (liquid injection) |
| Gas saver | On after 2 min |
| Concentration | 1.25-40 ppm |
| range (μg/ml) | |
| GC Column | HP-5MS UI 19091S 30 m × 250 μm × 0.25 μm |

*These values may vary depending on the column and the detector MS used

### Analysis of Alkanes and Instrumental GCMS Method Conditions

500 μl of sample (resuspended pellet or broth) was extracted with 500 μl of chloroform:methanol (1:2) for an hour at 1400 rpm, 30° C. 500 μl of hexane was added and extracted for one hour at 1400 rpm, 30° C. The samples were centrifuged for 30 minutes at 1,500×g and the top layer was transferred to an insert and was injected directly into the GCMS (1 μl). GCMS conditions are given in Table 6.

1000 ppm of stock of alkanes in hexane was used to prepare the substocks for a calibration curve.

TABLE 6
GCMS CONDITIONS

| | |
|---|---|
| PARAMETER | VALUE |

| Carrier Gas | Helium at constant flow (1.0 ml/min) | |
|---|---|---|
| Injector | Split ratio* | Split 5:1 |
| | Temperature | 250° C. |
| Detector | Source Temperature | 230° C. |
| | Quad Temperature | 150° C. |
| | Interface | 260° C. |
| | Gain | 1 |
| | Scan Range | m/z 50-600 |
| | Threshold | 150 |
| | A/D samples* | 2 |
| | Scan Speed* | 3125 (N = 1) |
| | Frequency (scans/sec)* | 5.1 |
| | Mode | SCAN and SIM |

| | |
|---|---|
| Solvent delay* | 5.0 min |
| Oven Temperature | Initial T: 60° C. × 1.00 min |
| Oven Ramp | 10° C./min to 325° C. for 10 min |
| Injection volume | 1 μl (liquid injection) |
| Gas saver | On after 2 min |
| Concentration range | 1.25-20 ppm |
| (μg/ml) | |
| GC Column | HP-5MS UI 19091S 30 m × |
| | 250 μm × 0.25 μm |

*These values may vary depending on the column and the detector MS used. Ions used for the quantitation in selected ion monitoring (SIM) acquisition mode (m/z) were 57, 71, 85. All the alkanes present the same fragmentation pattern and the ions used for the monitoring in the SIM method are the same. The only difference between alkanes is the molecular ion and their RT.

### Gene Expression on Adipate and Pimelate

Table 7 shows gene expression on adipate and pimelate relative to fructose using RNA sequence data.

TABLE 7

| | Expression on adipate | Expression on pimelate |
|---|---|---|
| Gene | relative to fructose | relative to fructose |
| B0198 | 8.1 | 0.95 |
| B0199 | 8.0 | 1.1 |
| B0200 | 7.8 | 1.1 |
| B0201 | 8.9 | 0.77 |
| B0202 | 10 | 1.1 |
| B1446 | — | — |
| B1447 | 11 | 7.2 |
| B1448 | 12 | 8.5 |
| B1449 | 10 | 6.3 |
| B2555 | 28 | 9.6 |
| A1526 | 3.0 | 1.8 |
| A1527 | 1.9 | 1.8 |
| A1528 | 3.3 | 1.8 |
| A1529 | 2.4 | 1.1 |
| A1530 | 3.0 | 1.2 |
| A1531 | — | — |
| A2818 | 2.9 | 28 |
| A0814 | 3.9 | 2.1 |
| A0815 | 3.6 | 2.1 |
| A0816 | 4.0 | 2.5 |
| A1067 | 3.2 | 1.4 |
| A1068 | 5.9 | 2.1 |
| A0459 | — | — |
| A0460 | 1.0 | 1.1 |
| A0461 | 1.1 | 0.9 |
| A0462 | — | — |
| A0463 | — | — |
| A0464 | — | — |
| A3350 | 0.93 | 15 |
| A3351 | 0.60 | 9.9 |
| A1519 | 0.89 | 3.2 |
| A1520 | 0.73 | 4.6 |

— RNA seq data too low for detection

### Sequence Information for Sequences in Sequence Listing

TABLE 8

| SEQ ID | |
|---|---|
| NO: | Sequence Description |
| 1 | Amino acid sequence of WP_011242364.1 MULTISPECIES: long-chain |
| | acyl-[acyl-carrier-protein] reductase [Synechococcus] |
| 2 | Nucleic acid sequence of WP_011242364.1 MULTISPECIES: long-chain |
| | acyl-[acyl-carrier-protein] reductase [Synechococcus] codon |
| | optimized |
| 3 | Amino acid sequence of WP_011378104.1 MULTISPECIES: aldehyde |
| | decarbonylase [Synechococcus] |
| 4 | Nucleic acid sequence of WP_011378104.1 MULTISPECIES: aldehyde |
| | decarbonylase [Synechococcus] codon optimized |
| 5 | Amino acid sequence of NP_415026.1 YBBO putative oxidoreductase |
| | [Escherichia coli str. K-12 substr. MG1655] |
| 6 | Nucleic acid sequence of NP_415026.1 YBBO putative oxidoreductase |
| | [Escherichia coli str. K-12 substr. MG1655] codon optimized |
| 7 | Amino acid sequence of NP_416319.1 acyl-CoA synthetase FADD(long- |
| | chain-fatty-acid--CoA ligase) [Escherichia coli str. K-12 substr. |
| | MG1655] |
| 8 | Nucleic acid sequence of NP_416319.1 acyl-CoA synthetase FADD(long- |
| | chain-fatty-acid--CoA ligase) [Escherichia coli str. K-12 substr. |
| | MG1655] codon optimized |
| 9 | Amino acid sequence of tr\|A6EVI7\|A6EVI7_9ALTE Putative |
| | dehydrogenase domain of multifunctional non-ribosomal peptide |
| | synthetases and related enzyme OS = Marinobacter algicola DG893 |
| | GN = MDG893_11561 PE = 4 SV = 1 |
| 10 | Nucleic acid sequence of tr\|A6EVI7\|A6EVI7_9ALTE Putative |
| | dehydrogenase domain of multifunctional non-ribosomal peptide |
| | synthetases and related enzyme OS = Marinobacter algicola DG893 |
| | GN = MDG893_11561 PE = 4 SV = 1 codon optimized |
| 11 | Amino acid sequence of tr\|Q1N697\|Q1N697_9GAMM Putative |
| | dehydrogenase domain of multifunctional non-ribosomal peptide |
| | synthetases and related enzyme OS = Bermanella marisrubri |
| | GN = RED65_09894 PE = 4 SV = 1 |
| 12 | Nucleic acid sequence of tr\|Q1N697\|Q1N697_9GAMM Putative |
| | dehydrogenase domain of multifunctional non-ribosomal peptide |
| | synthetases and related enzyme OS = Bermanella marisrubri |
| | GN = RED65_09894 PE = 4 SV = 1 codon optimized |
| 13 | Amino acid sequence of gi\|19551938\|ref\|NP_599940.1\|: 1-543 detergent |
| | sensitivity rescuer dtsR1 [Corynebacterium glutamicum ATCC 13032] |
| 14 | Nucleic acid sequence of gi\|19551938\|ref\|NP_599940.1\|: 1-543 |
| | detergent sensitivity rescuer dtsRl [Corynebacterium glutamicum |
| | ATCC 13032] codon optimized |
| 15 | Amino acid sequence of gi\|19551930\|ref\|NP_599932.1\|: 1-591 acyl-CoA |
| | carboxylase [Corynebacterium glutamicum ATCC 13032] |
| 16 | Nucleic acid sequence of gi\|19551930\|ref\|NP_599932.1\|: 1-591 acyl- |
| | CoA carboxylase [Corynebacterium glutamicum ATCC 13032] |
| 17 | Amino acid sequence of gi\|19551936\|ref\|NP_599938.1\|: 1-82 |
| | hypothetical protein NCg10676 [Corynebacterium glutamicum ATCC |
| | 13032] |
| 18 | Nucleic acid sequence of gi\|19551936\|ref\|NP_599938.1\|: 1-82 |
| | hypothetical protein NCg10676 [Corynebacterium glutamicum ATCC |
| | 13032] codon optimized |
| 19 | Amino acid sequence of WP_085050280.1 multifunctional acyl-CoA |
| | thioesterase I/protease I/lysophospholipase L1 ('tesA - |
| | truncated)[Escherichia coli] |
| 20 | Nucleic acid sequence of WP_085050280.1 multifunctional acyl-CoA |
| | thioesterase I/protease I/lysophospholipase L1 ('tesA - |
| | truncated)[Escherichia coli] |
| 21 | Amino acid sequence of TE, Weissella confusa LBAE C39-2, H1X5Q2 |
| 22 | Nucleic acid sequence of TE, Weissella confusa LBAE C39-2, H1X5Q2 |
| | codon optimized |
| 23 | Amino acid sequence of TE Clostridium argentinense CDC 2741, |
| | A0A0C1QZB7 |
| 24 | Nucleic acid sequence of TE Clostridium argentinense CDC 2741, |
| | A0A0C1QZB7 codon optimized |
| 25 | Amino acid sequence of TE Lactococcus raffinolactis 4877, I7KI30 |
| 26 | Nucleic acid sequence of TE Lactococcus raffinolactis 4877, I7KI30 |
| | codon optimized |
| 27 | Amino acid sequence of TE Petunia integrifolia subsp. inflata, |
| | Q6PUQ2 |
| 28 | Nucleic acid sequence of TE Petunia integrifolia subsp. inflata, |
| | Q6PUQ2 codon optimized |
| 29 | Amino acid sequence of TE Peptoniphilus harei ACS-146-V-Sch2b, |
| | E4L0C9 |
| 30 | Nucleic acid sequence of TE Peptoniphilus harei ACS-146-V-Sch2b, |
| | E4L0C9 codon optimized |
| 31 | Amino acid sequence of TE Clostridium botulinum (strain Okra/Type |
| | B1), B1IHP0 |
| 32 | Nucleic acid sequence of TE Clostridium botulinum (strain Okra/ |
| | Type B1), B1IHP0 codon optimized |
| 33 | Amino acid sequence of TE Spirochaeta smaragdinae (strain DSM 11293/ |
| | JCM 15392/SEBR 4228)E1RAP4 |
| 34 | Nucleic acid sequence of TE Spirochaeta smaragdinae (strain DSM |
| | 11293/JCM 15392/SEBR 4228)E1RAP4 codon optimized |
| 35 | Amino acid sequence of TE Eubacterium limosum (strain KIST612), |
| | E3GJ26 |
| 36 | Nucleic acid sequence of TE Eubacterium limosum (strain KIST612), |
| | E3GJ26 codon optimized |
| 37 | Amino acid sequence of TE Escherichia coli (strain K12), P0A8Z3 |
| 38 | Nucleic acid sequence of TE Escherichia coli (strain K12) , P0A8Z3 |
| | codon optimized |
| 39 | Amino acid sequence of TE Lactococcus lactis subsp. lactis (strain |
| | CV56), F2HJJ6 |
| 40 | Nucleic acid sequence of TE Lactococcus lactis subsp. lactis |
| | (strain CV56), F2HJJ6 codon optimized |
| 41 | Amino acid sequence of TE Clostridium sp. HMP27, A0A099RRK7 |
| 42 | Nucleic acid sequence of TE Clostridium sp. HMP27, A0A099RRK7 codon |
| | optimized |
| 43 | Amino acid sequence of TE Haemophilus influenzae (strain ATCC 51907/ |
| | DSM 11121/KW20/Rd), P44679 |
| 44 | Nucleic acid sequence of TE Haemophilus influenzae (strain ATCC |
| | 51907/DSM 11121/KW20/Rd), P44679 codon optimized |
| 45 | Amino acid sequence of TE Weissella paramesenteroides ATCC 33313, |
| | C5R921 |
| 46 | Nucleic acid sequence of TE Weissella paramesenteroides ATCC 33313, |
| | C5R921 codon optimized |
| 47 | Amino acid sequence of TE Clostridiales bacterium oral taxon 876 |
| | str. F0540, U2CXE7 |
| 48 | Nucleic acid sequence of TE Clostridiales bacterium oral taxon 876 |
| | str. F0540, U2CXE7 codon optimized |
| 49 | Amino acid sequence of TE Streptococcus mitis SPAR10, J0YTE5 |
| 50 | Nucleic acid sequence of TE Streptococcus mitis SPAR10, J0YTE5 |
| | codon optimized |
| 51 | Amino acid sequence of TE Bacteroides finegoldii CL09T03C10, K5D7V3 |
| 52 | Nucleic acid sequence of TE Bacteroides finegoldii CL09T03C10, |
| | K5D7V3 codon optimized |
| 53 | Amino acid sequence of TE Clostridium sp. CAG: 221, R6FXC3 |
| 54 | Nucleic acid sequence of TE Clostridium sp. CAG: 221, R6FXC3 codon |
| | optimized |
| 55 | Amino acid sequence of TE Solanum lycopersicum (Tomato) |
| | (Lycopersicon esculentum), B5B3P5 |
| 56 | Nucleic acid sequence of TE Solanum lycopersicum (Tomato) |
| | (Lycopersicon esculentum), B5B3P5 codon optimized |
| 57 | Amino acid sequence of TE Picea sitchensis (Sitka spruce) (Pinus |
| | sitchensis), A9NV70 |
| 58 | Nucleic acid sequence of TE Picea sitchensis (Sitka spruce) (Pinus |
| | sitchensis), A9NV70 codon optimized |
| 59 | Amino acid sequence of TE Pseudoramibacter alactolyticus ATCC |
| | 23263, E6MF99 |
| 60 | Nucleic acid sequence of TE Pseudoramibacter alactolyticus ATCC |
| | 23263, E6MF99 codon optimized |
| 61 | Amino acid sequence of TE Clostridium botulinum D str. 1873, C5VPS2 |
| 62 | Nucleic acid sequence of TE Clostridium botulinum D str. 1873, |
| | C5VPS2 codon optimized |
| 63 | Amino acid sequence of TE Bos taurus (Bovine), Q3B7M2 |
| 64 | Nucleic acid sequence of TE Bos taurus (Bovine), Q3B7M2 codon |
| | optimized |
| 65 | Amino acid sequence of TE Alkaliphilus oremlandii (strain OhILAs) |
| | (Clostridium oremlandii (strain OhILAs)), A8MEW2 |
| 66 | Nucleic acid sequence of TE Alkaliphilus oremlandii (strain OhILAs) |
| | (Clostridium oremlandii (strain OhILAs)), A8MEW2 codon optimized |
| 67 | Amino acid sequence of TE Desulfotomaculum nigrificans (strain DSM |
| | 14880/VKM B-2319/CO-1-SRB) (Desulfotomaculum carboxydivorans), |
| | F6B7F0 |
| 68 | Nucleic acid sequence of TE Desulfotomaculum nigrificans (strain |
| | DSM 14880/VKM B-2319/CO-1-SRB) (Desulfotomaculum |
| | carboxydivorans), F6B7F0 codon optimized |
| 69 | Amino acid sequence of TE Cellulosilyticum lentocellum (strain ATCC |
| | 49066/DSM 5427/NCIMB 11756/RHM5), F2JLT2 |
| 70 | Nucleic acid sequence of TE Cellulosilyticum lentocellum (strain |
| | ATCC 49066/DSM 5427/NCIMB 11756/RHM5), F2JLT2 codon optimized |
| 71 | Amino acid sequence of TE Paenibacillus sp. IHBB 10380, A0A0D3V4E9 |
| 72 | Nucleic acid sequence of TE Paenibacillus sp. IHBB 10380, |
| | A0A0D3V4E9 codon optimized |
| 73 | Amino acid sequence of TE Carboxydothermus hydrogenoformans (strain |
| | ATCC BAA-161/DSM 6008/Z-2901), Q3ADW4 |
| 74 | Nucleic acid sequence of TE Carboxydothermus hydrogenoformans |
| | (strain ATCC BAA-161/DSM 6008/Z-2901), Q3ADW4 codon optimized |
| 75 | Amino acid sequence of TE Clostridium carboxidivorans P7, C6Q1L2 |
| 76 | Nucleic acid sequence of TE Clostridium carboxidivorans P7, C6Q1L2 |
| | codon optimized |
| 77 | Amino acid sequence of TE Thermovirga lienii (strain ATCC BAA-1197/ |
| | DSM 17291/Cas60314), G7V8P3 |
| 78 | Nucleic acid sequence of TE Thermovirga lienii (strain ATCC BAA- |
| | 1197/DSM 17291/Cas60314), G7V8P3 codon optimized |
| 79 | Amino acid sequence of TE Selaginella moellendorffii (Spikemoss), |
| | D8QRX8 |
| 80 | Nucleic acid sequence of TE Selaginella moellendorffii (Spikemoss), |
| | D8QRX8 codon optimized |
| 81 | Amino acid sequence of TE Treponema caldarium (strain ATCC 51460/ |
| | DSM 7334/H1), F8F2E5 |
| 82 | Nucleic acid sequence of TE Treponema caldarium (strain ATCC 51460/ |
| | DSM 7334/H1), F8F2E5 codon optimized |
| 83 | rnpBT1 terminator sequence |
| 84 | Nucleic acid sequence for AAR gene together with oxidoreductase |
| | YbbO |

1: A process for the biosynthesis of compounds involved in fatty acid metabolism comprising:
obtaining an organism capable of producing compounds involved in fatty acid metabolism, derivatives thereof and/or compounds related thereto;
altering the organism; and
producing more compounds involved in fatty acid metabolism, derivatives thereof and/or compounds related thereto by the altered organism as compared to the unaltered organism.

2: The process of claim 1 wherein the organism is C. necator or an organism with properties similar thereto.

3: The process of claim 1 wherein the organism is altered by inserting a non-natural pathway to intercept fatty acyl-ACP intermediates.

4: The process of claim 3 wherein a thioesterase is inserted to generate free fatty acids and/or a fatty acyl-CoA reductase is inserted to generate fatty alcohols.

5. (canceled)

6: The process of claim 3 wherein an acyl-ACP reductase and/or aldehyde decarbonylase and/or oxidoreductase and/or acyl-CoA synthetase is inserted.

7: The process of claim 4 wherein the thioesterase is from Weissella confusa, Clostridium argentinense, Lactococcus raffinolactis, Petunia integrifolia, Peptoniphilus harei, Clostridium botulinum, Spirochaeta smaragdinae, Eubacterium limosum, Escherichia coli, Lactococcus lactis, Clostridium sp., Haemophilus influenzae, Weissella paramesenteroides, Clostridiales bacterium, Streptococcus mitis, Bacteroides finegoldii, Solanum lycopersicum, Picea sitchensis, Pseudoramibacter alactolyticus, Bos Taurus, Alkaliphilus oremlandii, Desulfotomaculum nigrificans, Cellulosilyticum lentocellum, Paenibacillus sp., Carboxydothermus hydrogenoformans, Clostridium carboxidivorans, Thermovirga lienii, Selaginella moellendorffii or Treponema caldarium and/or the fatty acyl-CoA reductase is from Bermanella marisrubri or Marinobacter algicola.

8: The process of claim 4 wherein the thioesterase comprises SEQ ID NO:19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 or 81 or a polypeptide with similar enzymatic activities exhibiting at least about 50% sequence identity to an amino acid sequence set forth in SEQ ID NO: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 or 81 or a functional fragment thereof or is encoded by a nucleic acid sequence comprising SEQ ID NO:20, 22, 24, 26, 28, 30, 32, 34, 36, 38, 40, 42, 44, 46, 48, 50, 52, 54, 56, 58, 60, 62, 64, 66, 68, 70, 72, 74, 76, 80 or 82 or a nucleic acid sequence encoding a polypeptide with similar enzymatic activities exhibiting at least about 50% sequence identity to the nucleic acid sequence set forth in SEQ ID NO: 20, 22, 24, 26, 28, 30, 32, 34, 36, 38, 40, 42, 44, 46, 48, 50, 52, 54, 56, 58, 60, 62, 64, 66, 68, 70, 72, 74, 76, 80 or 82 or a functional fragment thereof.

9-10. (canceled)

11: The process of claim 4 wherein the fatty acyl-CoA comprises SEQ ID NO: 9 or 11 or a polypeptide with similar enzymatic activities exhibiting at least about 50% sequence identity to an amino acid sequence set forth in SEQ ID NO: 9 or 11 or a functional fragment thereof or is encoded by a nucleic acid sequence comprising SEQ ID NO:10 or 12 or a nucleic acid sequence encoding a polypeptide with similar enzymatic activities exhibiting at least about 50% sequence identity to the nucleic acid sequence set forth in SEQ ID NO: 10 or 12 or a functional fragment thereof.

12. (canceled)

13: The process of claim 6 wherein the acyl-ACP reductase and/or aldehyde decarbonylase is from Synechococcus.

14: The process of claim 6 wherein the acyl-ACP reductase comprises SEQ ID NO:1 or a polypeptide with similar enzymatic activities exhibiting at least about 50% sequence identity to an amino acid sequence set forth in SEQ ID NO: 1 or a functional fragment thereof or is encoded by a nucleic acid sequence comprising SEQ ID NO:2 or a nucleic acid sequence encoding a polypeptide with similar enzymatic activities exhibiting at least about 50% sequence identity to the nucleic acid sequence set forth in SEQ ID NO: 2 or a functional fragment thereof.

15-16. (canceled)

17: The process of claim 6 wherein the aldehyde decarbonylase comprises SEQ ID NO:3 or a polypeptide with similar enzymatic activities exhibiting at least about 50% sequence identity to an amino acid sequence set forth in SEQ ID NO: 3 or a functional fragment thereof or is encoded by a nucleic acid sequence comprising SEQ ID NO:4 or a nucleic acid sequence encoding a polypeptide with similar enzymatic activities exhibiting at least about 50% sequence identity to the nucleic acid sequence set forth in SEQ ID NO: 4 or a functional fragment thereof.

18. (canceled)

19: The process of claim 6 wherein the oxidoreductase and/or acyl-CoA synthetase is from E. coli.

20: The process of claim 6 wherein the oxidoreductase comprises SEQ ID NO:5 or a polypeptide with similar enzymatic activities exhibiting at least about 50% sequence identity to an amino acid sequence set forth in SEQ ID NO: 5 or a functional fragment thereof or is encoded by a nucleic acid sequence comprising SEQ ID NO:6 or a nucleic acid sequence encoding a polypeptide with similar enzymatic activities exhibiting at least about 50% sequence identity to the nucleic acid sequence set forth in SEQ ID NO: 6 or a functional fragment thereof.

21-22. (canceled)

23: The process of claim 6 wherein the acyl-CoA synthetase comprises SEQ ID NO:7 or a polypeptide with similar enzymatic activities exhibiting at least about 50% sequence identity to an amino acid sequence set forth in SEQ ID NO: 7 or a functional fragment thereof or is encoded by a nucleic acid sequence comprising SEQ ID NO:8 or a nucleic acid sequence encoding a polypeptide with similar enzymatic activities exhibiting at least about 50% sequence identity to the nucleic acid sequence set forth in SEQ ID NO: 8 or a functional fragment thereof.

24. (canceled)

25: The process of claim 1 wherein the organism is further altered to delete one or more enzymes of the β-oxidation pathway.

26: The process of claim 25 wherein the fatty acid is pimelic acid or adipic acid.

27: The process of claim 26 wherein the fatty acid is pimelic acid and the organism is further altered to delete one or more enzymes which activate pimelate; further altered to inhibit acyl-CoA dehydrogenase; or further altered to delete a cluster selected from A0459-0464 (β-oxidation cluster 1) and A1526-1531 β-oxidation cluster 2).

28: The process of claim 27 wherein one or more genes selected from A3350-51 (acyl-CoA ligase and transport genes), A1519-20 (acyl-CoA ligase and transport genes), B1446-9 (acyl-CoA transferase, transport and regulatory gene), A2818 (glutaryl-CoA dehydrogenase gene), B2555 (acyl-CoA dehydrogenase gene) and A0814-16 (electron transfer and acyl-CoA dehydrogenase genes) are deleted.

29-31. (canceled)

32: The process of claim 26 wherein the fatty acid is adipic acid and the organism is further altered by deleting an adipic acid specific operon; deleting one or more enzymes which activate adipate; to inhibit acyl-CoA dehydrogenase; or to delete A0459-0464 (β-oxidation cluster 1).

33: The process of claim 32 wherein the adipic acid specific operon is B0198-202 (acyl-CoA transferase, thiolase, dehydrogenase and transport).

34. (canceled)

35: The process of claim 32 wherein B1446-9 (acyl-CoA transferase, transport and regulatory gene) is deleted.

36. (canceled)

37: The process of claim 32 wherein one or more genes selected from B2555 (acyl-CoA dehydrogenase gene), A1526-1531 (β-oxidation cluster 2), A2818 (glutaryl-CoA dehydrogenase gene), A0814-16 (electron transfer and acyl-CoA dehydrogenase genes) and A1067/68 (acyl-CoA dehydrogenase genes) is deleted.

38. (canceled)

39: The process of claim 1 wherein the organism is further altered to eliminate phaCAB, involved in PHBs production and/or H16-A0006-9 encoding endonucleases thereby improving transformation efficiency.

40. (canceled)

41: An altered organism capable of producing more compounds involved in fatty acid metabolism, derivatives thereof and/or compounds related thereto as compared to an unaltered organism.

42: The altered organism of claim 41 which is C. necator or an organism with properties similar thereto.

43: The altered organism of claim 41 comprising a non-natural pathway to intercept fatty acyl-ACP intermediates.

44: The altered organism of claim 41 wherein a thioesterase is inserted to generate free fatty acids and/or a fatty acyl-CoA reductase is inserted to generate fatty alcohols.

45. (canceled)

46: The altered organism of claim 41 wherein an acyl-ACP reductase and/or aldehyde decarbonylase and/or oxidoreductase and/or acyl-CoA synthetase is inserted to generate alka(e)nes.

47: The altered organism of claim 44 wherein the thioesterase is from Weissella confusa, Clostridium argentinense, Lactococcus raffinolactis, Petunia integrifolia, Peptoniphilus harei, Clostridium botulinum, Spirochaeta smaragdinae, Eubacterium limosum, Escherichia coli, Lactococcus lactis, Clostridium sp., Haemophilus influenzae, Weissella paramesenteroides, Clostridiales bacterium, Streptococcus mitis, Bacteroides finegoldii, Solanum lycopersicum, Picea sitchensis, Pseudoramibacter alactolyticus, Bos Taurus, Alkaliphilus oremlandii, Desulfotomaculum nigrificans, Cellulosilyticum lentocellum, Paenibacillus sp., Carboxydothermus hydrogenoformans, Clostridium carboxidivorans, Thermovirga lienii, Selaginella moellendorffii or Treponema caldarium and/or the fatty acyl-CoA reductase is from Bermanella marisrubri or Marinobacter algicola.

48: The altered organism of claim 44 wherein the thioesterase comprises SEQ ID NO:19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 or 81 or a polypeptide with similar enzymatic activities exhibiting at least about 50% sequence identity to an amino acid sequence set forth in SEQ ID NO: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 or 81 or a functional fragment thereof or is encoded by a nucleic acid sequence comprising SEQ ID NO: 20, 22, 24, 26, 28, 30, 32, 34, 36, 38, 40, 42, 44, 46, 48, 50, 52, 54, 56, 58, 60, 62, 64, 66, 68, 70, 72, 74, 76, 80 or 82 or a nucleic acid sequence encoding a polypeptide with similar enzymatic activities exhibiting at least about 50% sequence identity to the nucleic acid sequence set forth in SEQ ID NO: 20, 22, 24, 26, 28, 30, 32, 34, 36, 38, 40, 42, 44, 46, 48, 50, 52, 54, 56, 58, 60, 62, 64, 66, 68, 70, 72, 74, 76, 80 or 82 or a functional fragment thereof.

49-50. (canceled)

51: The altered organism of claim 44 wherein the fatty acyl-CoA comprises SEQ ID NO: 9 or 11 or a polypeptide with similar enzymatic activities exhibiting at least about 50% sequence identity to an amino acid sequence set forth in SEQ ID NO: 9 or 11 or a functional fragment thereof or is encoded by a nucleic acid sequence comprising SEQ ID NO: 10 or 12 or a nucleic acid sequence encoding a polypeptide with similar enzymatic activities exhibiting at least about 50% sequence identity to the nucleic acid sequence set forth in SEQ ID NO: 10 or 12 or a functional fragment thereof.

52. (canceled)

53: The altered organism of claim 46 wherein the acyl-ACP reductase and/or the aldehyde decarbonylase is from Synechococcus.

54: The altered organism of claim 46 wherein the acyl-ACP reductase comprises SEQ ID NO:1 or a polypeptide with similar enzymatic activities exhibiting at least about 50% sequence identity to an amino acid sequence set forth in SEQ ID NO: 1 or a functional fragment thereof or is encoded by a nucleic acid sequence comprising SEQ ID NO:2 or a nucleic acid sequence encoding a polypeptide with similar enzymatic activities exhibiting at least about 50% sequence identity to the nucleic acid sequence set forth in SEQ ID NO: 2 or a functional fragment thereof.

55-56. (canceled)

57: The altered organism of claim 46 wherein the aldehyde decarbonylase comprises SEQ ID NO:3 or a polypeptide with similar enzymatic activities exhibiting at least about 50% sequence identity to an amino acid sequence set forth in SEQ ID NO: 3 or a functional fragment thereof or is encoded by a nucleic acid sequence comprising SEQ ID NO:4 or a nucleic acid sequence encoding a polypeptide with similar enzymatic activities exhibiting at least about 50% sequence identity to the nucleic acid sequence set forth in SEQ ID NO: 4 or a functional fragment thereof.

58. (canceled)

59: The altered organism of claim 46 wherein the oxidoreductase and/or the acyl-CoA synthetase is from E. coli.

60: The altered organism of claim 46 wherein the oxidoreductase comprises SEQ ID NO:5 or a polypeptide with similar enzymatic activities exhibiting at least about 50% sequence identity to an amino acid sequence set forth in SEQ ID NO: 5 or a functional fragment thereof or is encoded by a nucleic acid sequence comprising SEQ ID NO:6 or a nucleic acid sequence encoding a polypeptide with similar enzymatic activities exhibiting at least about 50% sequence identity to the nucleic acid sequence set forth in SEQ ID NO: 6 or a functional fragment thereof.

61-62. (canceled)

63: The altered organism of claim 46 wherein the acyl-CoA synthetase comprises SEQ ID NO:7 or a polypeptide with similar enzymatic activities exhibiting at least about 50% sequence identity to an amino acid sequence set forth in SEQ ID NO: 7 or a functional fragment thereof or is encoded by a nucleic acid sequence comprising SEQ ID NO:8 or a nucleic acid sequence encoding a polypeptide with similar enzymatic activities exhibiting at least about 50% sequence identity to the nucleic acid sequence set forth in SEQ ID NO: 8 or a functional fragment thereof.

64. (canceled)

65: The altered organism of claim 41 wherein the organism is further altered to delete one or more enzymes of the β-oxidation pathway.

66: The altered organism of claim 65 wherein the fatty acid is pimelic acid or adipic acid.

67: The altered organism of claim 66 wherein the fatty acid is pimelic acid and the organism is further altered to delete one or more enzymes which activate pimelate; to inhibit acyl-CoA dehydrogenase; or to delete a cluster selected from A0459-0464 (β-oxidation cluster 1) and A1526-1531 (β-oxidation cluster 2).

68: The altered organism of claim 67 wherein one or more genes selected from A3350-51 (acyl-CoA ligase and transport genes), A1519-20 (acyl-CoA ligase and transport genes), B1446-9 (acyl-CoA transferase, transport and regulatory gene), A2818 (glutaryl-CoA dehydrogenase gene), B2555 (acyl-CoA dehydrogenase gene) and A0814-16 (electron transfer and acyl-CoA dehydrogenase genes) are deleted.

69-71. (canceled)

72: The altered organism of claim 66 wherein the fatty acid is adipic acid and the organism is further altered by deleting an adipic acid specific operon; to delete one or more enzymes which activate adipate; to inhibit acyl-CoA dehydrogenase; or to delete A0459-0464 (β-oxidation cluster 1).

73: The altered organism of claim 72 wherein the adipic acid specific operon is B0198-202 (acyl-CoA transferase, thiolase, dehydrogenase and transport).

74. (canceled)

75: The altered organism of claim 72 wherein B1446-9 (acyl-CoA transferase, transport and regulatory gene) is deleted.

76. (canceled)

77: The altered organism of claim 72 wherein one or more genes selected from B2555 (acyl-CoA dehydrogenase gene), A1526-1531 (β-oxidation cluster 2), A2818 (glutaryl-CoA dehydrogenase gene), A0814-16 (electron transfer and acyl-CoA dehydrogenase genes) and A1067/68 (acyl-CoA dehydrogenase genes) is deleted.

78. (canceled)

79: The altered organism of claim 41 wherein the organism is further altered to eliminate phaCAB, involved in PHBs production and/or H16-A0006-9 encoding endonucleases thereby improving transformation efficiency.

80. (canceled)

81: A bio-derived, bio-based, or fermentation-derived product produced from the method of claim 1, wherein said product comprises:
(i) a composition comprising at least one bio-derived, bio-based, or fermentation-derived compound or any combination thereof;
(ii) a molded substance obtained by molding the bio-derived, bio-based, or fermentation-derived composition or compound of (i); or
(iii) a bio-derived, bio-based, or fermentation-derived semi-solid or a non-semi-solid stream, comprising the bio-derived, bio-based, or fermentation-derived composition or compound of (i) or the bio-derived, bio-based, or fermentation-derived molded substance of (ii), or any combination thereof.

82: A bio-derived, bio-based or fermentation derived product produced in accordance with the central metabolism depicted in FIG. 1, 7 or 8.

83: An exogenous genetic molecule of the altered organism of claim 41.

84: The exogenous genetic molecule of claim 83 comprising a codon optimized nucleic acid sequence or an expression construct or synthetic operon of one or more enzymes of a non-natural pathway to intercept fatty acyl-ACP intermediates.

85: The exogenous genetic molecule of claim 84 codon optimized for C. necator.

86: The exogenous genetic molecule of claim 83 comprising a codon optimized nucleic acid sequence encoding one or more enzymes of a non-natural pathway to intercept fatty acyl-ACP intermediates.

87: The exogenous genetic molecule of claim 83 comprising a codon optimized nucleic acid sequence, expression construct or synthetic operon encoding a thioesterase, a fatty acyl-CoA reductase, an acyl-ACP reductase, an aldehyde decarbonylase, an oxidoreductase and/or an acyl-Co synthetase.

88-89. (canceled)

90: A process for the biosynthesis of compounds involved in fatty acid metabolism, said process comprising providing a means capable of producing compounds involved in fatty acid metabolism and producing compounds involved in fatty acid metabolism with said means.

91: A process for biosynthesis of compounds involved in fatty acid metabolism, and derivatives thereof, and compounds related thereto, said process comprising:
a step for performing a function of altering an organism capable of producing compounds involved in fatty acid metabolism, derivatives thereof, and/or compounds related thereto such that the altered organism produces more compounds involved in fatty acid metabolism, derivatives thereof, and/or compounds compared to a corresponding unaltered organism; and
a step for performing a function of producing compounds involved in fatty acid metabolism, derivatives thereof, and/or compounds related thereto in the altered organism.

92-93. (canceled)


SEQUENCE LISTING (84 sequences)

SEQ ID NO: 1 - protein, 341 aa, Synechococcus sp.
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALAI

SEQ ID NO: 2 - DNA, 1026 bp, Artificial sequence
  Synthetic
atgttcggactgattggccatttgacaagcttagaacaagcacgtgacgttagcagacgcatgggctacgacgaatacgcggaccagggcctggagttctggtcctccgcaccgccccagatcgtggatgagatcacggtcacctcggcgacgggcaaagtgatccacgggcgctatatcgaatcgtgcttcctgccggaaatgctggccgcccgccgcttcaagactgccacccgcaaggtcctgaacgccatgtcgcacgcgcagaagcacggcatcgacatctcggccttgggcggcttcacgtcgattatcttcgagaacttcgatctggcctccctgcgccaggtgcgcgacaccacgctggagttcgaacggttcacgacgggcaacacacacaccgcgtacgtgatctgccgccaggtcgaagcggcagcgaaaacgttggggatcgacatcacccaggccaccgtcgccgtggtgggcgcgaccggcgacatcggctcggccgtgtgccggtggctggacctgaagctgggcgtcggtgacctcatcctgaccgcccgcaaccaggaacgtctggacaatctgcaggccgagctcggccgcggcaagattctcccgctcgaagccgccctgcctgaggcagactttatcgtgtgggtggcgtcgatgccgcagggcgtggtgatcgatccggccaccctgaagcaaccgtgcgtgttgatcgacggtggctacccgaagaacctcggcagcaaggtccagggcgaaggcatctatgtcctgaacggtggcgtggtcgagcattgctttgacatcgactggcaaatcatgagcgcggccgagatggcccgcccggagcggcagatgttcgcgtgcttcgccgaggccatgctgctggagttcgagggctggcataccaatttctcctggggccgcaaccaaatcaccatcgaaaaaatggaagcgatcggtgaagcgagcgtccgccacggctttcagcccctcgcgctggccatctga

SEQ ID NO: 3 - protein, 231 aa, Synechococcus sp.
MPQLEASLELDFQSESYKDAYSRINAIVIEGEQEAFDNYNRLAEMLPDQRDELHKLAKMEQRHMKGFMACGKNLSVTPDMGFAQKFFERLHENFKAAAAEGKVVTCLLIQSLIIECFAIAAYNIYIPVADAFARKITEGVVRDEYLHRNFGEEWLKANFDASKAELEEANRQNLPLVWLMLNEVADDARELGMERESLVEDFMIAYGEALENIGFTTREIMRMSAYGLAAV

SEQ ID NO: 4 - DNA, 696 bp, Artificial sequence
  Synthetic
atgccacaactggaagcttcgctcgaattagattttcaatcggaatcatacaaggacgcctacagccgcatcaacgcaatcgtcatcgagggcgagcaagaagccttcgacaactacaaccggctggccgagatgctcccggatcagcgcgacgaactccacaaactggcgaaaatggaacagcgccacatgaagggcttcatggcgtgcggcaagaatctgtccgtcacgcccgacatgggcttcgcccagaagttcttcgagcgcctgcatgaaaacttcaaggcagccgcggccgagggcaaggtcgtgacgtgcctgctgatccagtccctgatcatcgagtgcttcgccatcgcggcgtacaacatctacattccggtggccgacgcgtttgcccgcaagatcaccgaaggcgtggtccgcgacgagtatctgcaccgcaacttcggcgaggaatggctgaaggccaacttcgacgcctcgaaggccgagttggaagaggccaaccgccagaatctgccgctggtgtggttgatgctgaacgaagtggcggacgacgcgcgtgaactgggcatggaacgcgagagcctcgtggaagatttcatgatcgcgtacggtgaggccctggagaatatcgggttcaccacccgcgagatcatgcggatgagcgcgtatggcctggcagcggtgtga

SEQ ID NO: 5 - protein, 269 aa, Escherichia coli
MTHKATEILTGKVMQKSVLITGCSSGIGLESALELKRQGFHVLAGCRKPDDVERMNSMGFTGVLIDLDSPESVDRAADEVIALTDNCLYGIFNNAGFGMYGPLSTISRAQMEQQFSANFFGAHQLTMRLLPAMLPHGEGRIVMTSSVMGLISTPGRGAYAASKYALEAWSDALRMELRHSGIKVSLIEPGPIRTRFTDNVNQTQSDKPVENPGIAARFTLGPEAVVDKVRHAFISEKPKMRYPVTLVTWAVMVLKRLLPGRVMDKILQG

SEQ ID NO: 6 - DNA, 810 bp, Artificial sequence
  Synthetic
atgacccacaaagcgactgaaatcttgaccggcaaagtgatgcaaaagtccgtcctgatcaccggctgctccagcgggatcggcctggagtccgcgctggaactcaagcgccagggcttccatgtgctggccgggtgccggaagcccgatgatgtcgagcgcatgaatagcatgggcttcaccggtgtgctcattgacctggactcgccggagtccgtggaccgcgccgcggacgaagtgatcgccctgacggacaactgcctgtacggcatcttcaacaacgccggctttggcatgtacggcccgctgtcgaccatcagccgtgcgcagatggaacagcaattcagcgcgaacttcttcggcgcacatcagctgacaatgcgcctgctgccggccatgctcccgcacggcgagggccgcatcgtgatgacctcgtcggtgatgggcctgatctcgacgcccggtcggggcgcctacgcagcatcgaagtatgcgctggaagcctggagcgacgcgctgcgcatggaactgcgccactcgggcatcaaagtgtcgctgatcgagccaggcccgatccgcacgcgcttcacggacaacgtcaaccagacccagagcgataagcccgtcgagaatccgggcatcgccgcgcgcttcaccttgggccctgaagccgtcgtggacaaggtccgccacgccttcatcagcgagaagcccaagatgcgttatccggtgacgctcgtgacctgggccgtcatggtgctcaagcggctgctgccggggcgcgtcatggacaagattctgcagggctga

SEQ ID NO: 7 - protein, 561 aa, Escherichia coli
MKKVWLNRYPADVPTEINPDRYQSLVDMFEQSVARYADQPAFVNMGEVMTFRKLEERSRAFAAYLQQGLGLKKGDRVALMMPNLLQYPVALFGILRAGMIVVNVNPLYTPRELEHQLNDSGASAIVIVSNFAHTLEKVVDKTAVQHVILTRMGDQLSTAKGTVVNFVVKYIKRLVPKYHLPDAISFRSALHNGYRMQYVKPELVPEDLAFLQYTGGTTGVAKGAMLTHRNMLANLEQVNATYGPLLHPGKELVVTALPLYHIFALTINCLLFIELGGQNLLITNPRDIPGLVKELAKYPFTAITGVNTLFNALLNNKEFQQLDFSSLHLSAGGGMPVQQVVAERWVKLTGQYLLEGYGLTECAPLVSVNPYDIDYHSGSIGLPVPSTEAKLVDDDDNEVPPGQPGELCVKGPQVMLGYWQRPDATDEIIKNGWLHTGDIAVMDEEGFLRIVDRKKDMILVSGFNVYPNEIEDVVMQHPGVQEVAAVGVPSGSSGEAVKIFVVKKDPSLTEESLVTFCRRQLTGYKVPKLVEFRDELPKSNVGKILRRELRDEARGKVDNKA

SEQ ID NO: 8 - DNA, 1686 bp, Artificial sequence
  Synthetic
atgaaaaaagtgtggctgaacagatatcccgcagacgtccctaccgagatcaacccagaccgctaccagtccctcgtggacatgtttgagcaatcggtcgcccgctatgcggatcagccggccttcgtgaatatgggtgaagtcatgacgtttcgtaagctggaagaacgcagccgtgccttcgcagcgtacttgcagcagggcctcggcctgaagaagggcgaccgcgtggccctgatgatgcccaatctgctgcagtaccctgtggccctgtttggcatcctgcgggcggggatgatcgtcgtcaacgtcaacccgctgtacaccccgcgcgagctggagcatcagctcaacgactccggcgcctcggccatcgtcatcgtgagcaacttcgcccataccctggagaaagtcgtcgataagaccgcggtccagcatgtgatcctgacgcgcatgggcgatcagctgagcaccgcgaagggcaccgtggtgaacttcgtggtgaagtatatcaagcgcctcgtgccgaagtaccatctgccggacgcgatttcgttccgctcggccctgcacaatggctaccgcatgcagtacgtcaagccggaactcgtgccagaggacctggcattcctgcagtacacgggcggcaccacgggcgtcgccaagggcgcgatgctgacccaccgcaacatgctcgcgaacctggaacaggtcaacgccacgtatggcccgctgctgcacccgggtaaggaactggtcgtgactgcgttgccgctctaccacattttcgccctgacaatcaactgcctcctcttcatcgagctgggcgggcaaaacctcttgatcacgaaccctcgcgatatccccggcctggtgaaggaactggccaagtacccctttacagcgatcaccggcgtcaacaccctcttcaacgccctgctgaataacaaagagttccagcagctggacttcagcagcctccacctgagcgcgggtggcggcatgcccgtccagcaagtcgtggcggagcgttgggtgaagctcacgggccagtatctgctggaaggctacggtctgacggaatgcgcgccgctggtgtcggtcaatccgtatgacatcgactaccactcgggctccattggcctgccggtcccgtcgactgaagcgaagctcgtggacgacgacgataatgaagtgccgccgggccagcccggggaattgtgcgtgaagggtccccaggtcatgctgggctactggcaacgcccggacgccaccgacgagatcatcaagaacggctggctgcacacgggcgacatcgccgtgatggacgaagagggtttcctccgcatcgtcgaccggaagaaagacatgatcctggtgagcggcttcaacgtctacccgaacgaaatcgaagatgtggtcatgcagcatccgggcgtgcaggaagtcgccgccgtgggcgtgccatcgggcagctcgggcgaggcggtcaaaatttttgtggtgaaaaaggacccgtcgctgaccgaagagtccctggtgaccttctgtcgccgccagctgacgggctataaggtcccgaagctcgtcgagttccgcgacgaattgcccaagagcaacgtcggcaagatcctgcgccgggagctgcgcgatgaagcgcgtggcaaggtggataacaaagcgtga

SEQ ID NO: 9 - protein, 512 aa, Artificial sequence
  Synthetic
MATQQQQNGASASGVLEQLRGKHVLITGTTGFLGKVVLEKLIRTVPDIGGIHLLIRGNKRHPAARERFLNEIASSSVFERLRHDDNEAFETFLEERVHCITGEVTESRFGLTPERFRALAGQVDAFINSAASVNFREELDKALKINTLCLENVAALAELNSAMAVIQVSTCYVNGKNSGQITESVIKPAGESIPRSTDGYYEIEELVHLLQDKISDVKARYSGKVLEKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLMAEAKTNYAAYDQLFYRRPTKPFVAVNRKLFDVVVGGMRVPLSIAGKAMRLAGQNRELKVLKNLDTTRSLATIFGFYTAPDYIFRNDSLMALASRMGELDRVLFPVDARQIDWQLYLCKIHLGGLNRYALKERKLYSLRAADTRKKAA

SEQ ID NO: 10 - DNA, 1539 bp, Artificial sequence
  Synthetic
atggccacacaacaacaacagaacggagcaagcgcgtcgggggtgttagaacagctgcgcggcaaacacgtgttgatcaccggcacgaccggctttctcggcaaagtcgtgctggaaaagttgatccggaccgtgcccgacatcggcggcatccacctgttgatccgcggcaacaagcgccaccccgccgcacgcgaacgcttcctcaacgaaatcgccagctcctcggtgttcgaacggctgcggcacgatgacaatgaagccttcgaaaccttcctggaagaacgtgtgcattgcatcaccggtgaggtgaccgaaagccgcttcggcctgaccccggagcggttccgcgccctggcgggtcaggtcgatgccttcatcaatagcgcggcatcggtcaacttccgcgaggagctcgacaaggccctgaagatcaacaccctctgcctggagaacgtcgccgcgctggccgagctgaacagcgcgatggcagtcatccaggtgtcgacgtgctatgtgaacggtaagaactccggtcaaatcaccgaatcggtgatcaagccggccggcgaatccatcccgcgcagcaccgacgggtactacgagatcgaagaactggtccatctgctccaggacaaaatttccgacgtcaaggcacgctacagcggcaaggtcttggagaagaagctcgtggatctgggcatccgcgaggccaacaactacggctggtccgacacttatacgttcacgaagtggctgggggaacagttgctgatgaaggccctctccggccgcagcctgacgattgtgcgcccgtcgatcatcgagtcggccctggaggaaccgtcgccgggctggatcgaaggcgtcaaggtcgccgacgccatcatcctcgcgtacgcgcgcgaaaaagtgtccctgttccccgggaagcgctcgggcatcatcgacgtgatccctgtcgacctggtcgccaactcgatcatcctgtcgctggcagaggccctcagcggctcgggccagcgtcgcatttaccagtgctgcagcggtgggagcaaccccatcagcctgggcaagttcattgactatctgatggcagaagccaagacgaactatgccgcctacgaccaactgttctaccgtcgcccgacgaagccgttcgtggccgtgaatcgcaagctgtttgatgtggtcgtgggcggcatgcgcgtgccgctcagcatcgcgggcaaggccatgcgcctggccgggcagaaccgcgagctcaaggtcctgaagaatctggacaccacacggtcgctggcgaccatcttcggcttctatacggcgcccgattatatcttccggaatgactcgctgatggcgctggcatcgcgcatgggcgagctggatcgcgtcctcttcccagtcgacgcgcgccagatcgactggcagctgtacctgtgcaagatccatctgggcgggctgaaccgctatgcgctcaaagagcgcaagctctatagcctgcgcgcggcggacacccgcaagaaagccgcctga

SEQ ID NO: 11 - protein, 514 aa, Artificial sequence
  Synthetic
MSQYSAFSVSQSLKGKHIFLTGVTGFLGKAILEKLLYSVPQLAQIHILVRGGKVSAKKRFQHDILGSSIFERLKEQHGEHFEEWVQSKINLVEGELTQPMFDLPSAEFAGLANQLDLIINSAASVNFRENLEKALNINTLCLNNIIALAQYNVAAQTPVMQISTCYVNGFNKGQINEEVVGPASGLIPQLSQDCYDIDSVFKRVHSQIEQVKKRKTDIEQQEQALIKLGIKTSQHFGWNDTYTFTKWLGEQLLIQKLGKQSLTILRPSIIESAVREPAPGWVEGVKVADALIYAYAKGRVSIFPGRDEGILDVIPVDLVANAAALSAAQLMESNQQTGYRIYQCCSGSRNPIKLKEFIRHIQNVAQARYQEWPKLFADKPQEAFKTVSPKRFKLYMSGFTAITWAKTIIGRVFGSNAASQHMLKAKTTASLANIFGFYTAPNYRFSSQKLEQLVKQFDTTEQRLYDIRADHFDWKYYLQEVHMDGLHKYALADRQELKPKHVKKRKRETIRQAA

SEQ ID NO: 12 - DNA, 1545 bp, Artificial sequence
  Synthetic
atgtcccagtacagcgccttttccgtttcgcagtccctcaaaggtaagcatatctttctgaccggcgtgacgggtttcctgggcaaggcaatcctggaaaagctgctgtactcggtcccgcagctcgcgcagatccacatcttggtccggggtggcaaggtgagcgccaagaaacgcttccagcacgacatcctggggagcagcatcttcgagcgcctgaaggaacagcacggggaacactttgaggaatgggtgcaatccaagatcaacctggtcgagggcgaactgacccagccaatgttcgatttgccgtcggccgagttcgcggggctcgcgaatcagttggatctgatcattaactccgcggcaagcgtgaacttccgcgaaaacctggaaaaggccctgaacattaatacgctctgtctgaacaacatcatcgccctcgcgcagtataacgtcgcggcccagacgcctgtgatgcaaatctccacgtgctatgtgaacggtttcaataagggccagatcaacgaagaagtggtgggtccggcgagcggcctgatcccccagctctcgcaggactgctacgacatcgacagcgtgttcaagcgcgtccattcgcagattgaacaggtcaagaagcgtaagaccgacatcgagcaacaggaacaagcgctcatcaagctcggcattaagacctcccaacacttcggctggaatgacacctacacgttcaccaagtggctcggggagcaactgctgatccagaagctcggcaagcagagcctgaccatcctgcgcccctcgattatcgagtcggcggtccgcgagccggccccgggctgggtcgagggcgtcaaagtcgcggacgccctgatctacgcctatgcgaagggccgggtgtcgattttccccgggcgcgacgaaggcatcctggatgtgatcccggtcgacctggtggcgaatgccgccgcactgagcgccgcgcagctgatggaatccaaccagcagaccggctatcgcatctaccagtgctgctcgggcagccgcaacccgatcaagctgaaggagttcatccggcacatccaaaatgtggcccaggcacgctaccaagagtggccaaagctgttcgcggacaaaccgcaggaagccttcaagaccgtgagcccgaagcgctttaagctgtacatgagcggcttcacagcgatcacgtgggccaagactatcatcggccgcgtctttggtagcaacgccgcctcgcagcacatgctgaaggccaagaccaccgcgtcgctggccaatatcttcggcttctacaccgcaccgaactaccgcttctcgtcgcagaaactggagcaactcgtgaagcaattcgatacgaccgaacagcgcctgtacgacatccgcgccgaccatttcgactggaagtattacctccaagaggtgcacatggacggcttgcacaagtacgcgctggccgatcgccaagaactgaagcccaaacacgtcaagaagcggaagcgtgaaacgatccggcaggccgcctga

SEQ ID NO: 13 - protein, 543 aa, Corynebacterium glutamicum
MTISSPLIDVANLPDINTTAGKIADLKARRAEAHFPMGEKAVEKVHAAGRLTARERLDYLLDEGSFIETDQLARHRTTAFGLGAKRPATDGIVTGWGTIDGREVCIFSQDGTVFGGALGEVYGEKMIKIMELAIDTGRPLIGLYEGAGARIQDGAVSLDFISQTFYQNIQASGVIPQISVIMGACAGGNAYGPALTDFVVMVDKTSKMFVTGPDVIKTVTGEEITQEELGGATTHMVTAGNSHYTAATDEEALDWVQDLVSFLPSNNRSYAPMEDFDEEEGGVEENITADDLKLDEIIPDSATVPYDVRDVIECLTDDGEYLEIQADRAENVVIAFGRIEGQSVGFVANQPTQFAGCLDIDSSEKAARFVRTCDAFNIPIVMLVDVPGFLPGAGQEYGGILRRGAKLLYAYGEATVPKITVTMRKAYGGAYCVMGSKGLGSDINLAWPTAQIAVMGAAGAVGFIYRKELMAADAKGLDTVALAKSFEREYEDHMLNPYHAAERGLIDAVILPSETRGQISRNLRLLKHKNVTRPARKHGNMPL

SEQ ID NO: 14 - DNA, 1632 bp, Artificial sequence
  Synthetic
atgaccatctcctccccgctgatcgacgtggccaacctcccggatatcaacaccacggccggcaagatcgccgatctgaaggcccgccgggccgaggcccatttcccgatgggcgaaaaggccgtggaaaaggtgcatgccgccggccgcctgacggcgcgcgagcgcctggactatctgctcgacgaaggctcgtttatcgaaaccgaccagctcgcgcggcatcgcaccacggccttcggcctcggcgcgaagcgccccgcgaccgacggcatcgtcacgggctggggcaccatcgacgggcgcgaggtctgcatcttctcccaagacgggaccgtgttcgggggcgcgctgggcgaggtgtacggggagaagatgatcaagatcatggaactcgccatcgacaccgggcgccccctgatcggcctgtacgaaggcgccggcgcgcgcatccaagacggcgccgtgtcgctggacttcatcagccagaccttctaccagaacatccaggcgagcggcgtcatcccgcagatcagcgtcatcatgggcgcctgcgcgggcggcaatgcgtacggcccggcgctgacggatttcgtggtcatggtggacaagacctcgaagatgttcgtgacgggccccgatgtgatcaagaccgtgacgggcgaagagatcacgcaagaagaactggggggcgccaccacccacatggtgaccgcgggcaactcgcactacaccgccgccacggacgaagaagccctggactgggtgcaggatctcgtcagctttctgccgagcaacaaccggagctatgcgccgatggaggacttcgacgaagaagagggcggcgtggaagagaacatcaccgcggacgacctgaagctggacgagattatcccggactcggccaccgtgccgtacgacgtgcgggatgtgatcgagtgcctgaccgacgacggcgagtacctggagattcaggccgatcgcgccgagaatgtcgtgatcgcgttcggccgcattgagggccagtcggtcggctttgtggccaaccagccgacccagttcgcgggctgcctggacattgattcgtcggagaaagccgcgcgcttcgtccgcacgtgcgacgcgttcaacatccccatcgtgatgctggtcgatgtgccgggcttcctgccgggcgcgggccaggaatacggcggcatcctgcgccgcggcgccaagctgctgtatgcgtatggcgaggcgaccgtcccgaagatcaccgtcaccatgcggaaggcctacggcggcgcctattgcgtgatgggcagcaagggcctgggcagcgacatcaacctggcgtggcccacggcccagatcgccgtgatgggcgccgccggcgccgtgggcttcatctaccggaaggaactgatggcggcggacgcgaagggcctggatacggtcgccctggccaagtcgtttgagcgcgagtacgaagatcacatgctgaacccctatcacgcggcggagcgcggcctgatcgacgccgtgatcctgccgtccgaaacgcgggggcagattagccgcaatctgcgcctgctgaagcacaaaaacgtgacccgcccggcgcgcaagcacggcaatatgccgctgtga

SEQ ID NO: 15 - protein, 591 aa, Corynebacterium glutamicum
MSVETRKITKVLVANRGEIAIRVFRAARDEGIGSVAVYAEPDADAPFVSYADEAFALGGQTSAESYLVIDKIIDAARKSGADAIHPGYGFLAENADFAEAVINEGLIWIGPSPESIRSLGDKVTARHIADTAKAPMAPGTKEPVKDAAEVVAFAEEFGLPIAIKAAFGGGGRGMKVAYKMEEVADLFESATREATAAFGRGECFVERYLDKARHVEAQVIADKHGNVVVAGTRDCSLQRRFQKLVEEAPAPFLTDDQRERLHSSAKAICKEAGYYGAGTVEYLVGSDGLISFLEVNTRLQVEHPVTEETTGIDLVREMFRIAEGHELSIKEDPAPRGHAFEFRINGEDAGSNFMPAPGKITSYREPQGPGVRMDSGVVEGSEISGQFDSMLAKLIVWGDTREQALQRSRRALAEYVVEGMPTVIPFHQHIVENPAFVGNDEGFEIYTKWIEEVWDNPIAPYVDASELDEDEDKTPAQKVVVEINGRRVEVALPGDLALGGTAGPKKKAKKRRAGGAKAGVSGDAVAAPMQGTVIKVNVEEGAEVNEGDTVVVLEAMKMENPVKAHKSGTVTGLTVAAGEGVNKGVVLLEIK

SEQ ID NO: 16 - DNA, 1776 bp, Artificial sequence
  Synthetic
atgagcgtcgaaacccgcaagatcaccaaggtcctggtggccaatcgcggcgagatcgccatccgcgtgttccgggcggcccgcgacgaaggcatcggcagcgtggccgtgtacgccgaacccgatgccgacgccccgttcgtgtcctatgccgacgaagcgttcgcgctgggcggccagaccagcgccgagagctatctggtcatcgataagatcatcgatgcggcgcgcaagtcgggcgccgacgcgatccaccccggctacgggtttctggccgagaacgccgactttgcggaagcggtgatcaacgaagggctgatctggattggcccgagcccggagtcgatccgcagcctcggcgataaggtcaccgcccgccacatcgcggacaccgccaaggcgccgatggcccccggcaccaaggaacccgtgaaggacgcggccgaagtcgtggcgttcgccgaagagttcggcctgccgatcgcgatcaaggccgcgtttggcggcggcggccgggggatgaaagtcgcctataagatggaagaagtggcggacctgttcgagtcggccacccgcgaggcgacggccgccttcggccggggcgagtgcttcgtggagcgctacctggacaaggcccggcacgtcgaggcccaggtcatcgccgataagcacggcaacgtcgtcgtggccggcacccgcgactgcagcctgcagcgccgcttccagaagctcgtggaagaggccccggccccgttcctgaccgacgaccagcgcgagcgcctgcacagctccgccaaggccatctgcaaagaagcgggctactacggggccggcaccgtggagtatctggtgggctccgacggcctgatctccttcctggaagtcaacacccgcctgcaagtcgaacacccggtgaccgaggaaacgacgggcattgacctggtgcgcgagatgttccgcatcgccgagggccatgagctgagcattaaagaagatccggcgccgcgcggccatgcgttcgagttccgcatcaacggcgaagatgccggctccaacttcatgccggcgccggggaagatcacctcgtaccgcgagccccagggccccggcgtgcggatggactcgggggtggtcgaaggcagcgaaatctcggggcagttcgactcgatgctggccaagctgattgtctggggcgacacgcgcgaacaggcgctgcagcggtcccgccgcgccctcgcggagtacgtggtcgagggcatgcccacggtgatcccgttccaccaacatatcgtggagaacccggcgttcgtcgggaacgacgaagggtttgaaatctacaccaagtggatcgaagaagtgtgggataaccccatcgcgccgtacgtggacgccagcgagctggacgaagatgaggacaagaccccggcgcagaaagtcgtggtggagatcaacgggcgccgcgtggaagtcgccctccccggcgacctggcgctgggcggcacggccggccccaagaaaaaggccaagaagcgccgggcgggcggcgccaaggccggcgtgtcgggcgacgcggtggccgcgccgatgcagggcacggtgatcaaggtgaacgtcgaagagggcgccgaggtcaatgaaggcgacaccgtggtggtcctggaagccatgaagatggagaatccggtgaaggcgcacaagagcggcacggtcacgggcctgacggtggccgccggcgagggcgtgaataaaggcgtggtcctgctcgaaatcaagtga

SEQ ID NO: 17 - protein, 82 aa, Corynebacterium glutamicum
MSEETTQDTKAAEKPFLQIVSGNPTDQEVAALTVVFAGLAKAAAAQQMVSASKDRNNWGNLDERLSRPNTFNPSAFQNVNFF

SEQ ID NO: 18 - DNA, 249 bp, Artificial sequence
  Synthetic
atgagcgaggaaacgacccaggacaccaaggccgccgagaagccgttcctgcagatcgtgagcggcaacccgaccgaccaagaagtggcggcgctgaccgtggtctttgcgggcctcgcgaaggccgccgccgcgcagcagatggtgtcggcctcgaaggaccgcaacaactggggcaatctggatgagcgcctgtcgcggccgaacacgttcaatccctccgccttccagaacgtcaacttcttctga

SEQ ID NO: 19 - protein, 183 aa, Escherichia coli
MADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDS

SEQ ID NO: 20 - DNA, 552 bp, Artificial sequence
  Synthetic
atggccgataccctgctgatcctgggcgactcgctgtcagccggctatcgcatgtcggcctcggccgcctggccggccctgctgaacgataagtggcagagcaagacctcggtggtgaacgcctcgatctcgggtgatacctcgcagcagggcctggcccgcctgccggcactgctgaaacagcatcagccacgctgggtgttggtggaactgggcggcaatgatggtctgcgcggcttccagccgcagcagaccgagcagaccctgcgccagatcttgcaggacgtgaaggccgccaacgccgaaccgctgctgatgcagatccgcctgccggccaactatggccgccgctacaacgaggccttctcggccatctacccgaagctggccaaggagttcgacgtgccgctgctgccgttcttcatggaggaggtgtacctgaagccgcagtggatgcaggacgacggcatccacccgaaccgcgacgcccagccgttcatcgccgactggatggccaagcagctgcagccgctggtgaaccacgactcgtga

SEQ ID NO: 21 - protein, 244 aa, Weissella confusa
MYSMQHEVLYYEADVTGKLSLPMIFNLAVLSSTQQSVDLGVGPDYAHANGVGWIILQHVVDIKRRPKIGEKVALETLAKEFNPFFAKRLYRIVDEAGNELVSIDALYAMIDMEKRKMARIPQEMVDAYAPERVKKIPRQPEPDHMIGDIPVDVDQQYAVRYLDIDSNRHVNNSKYFDWMQDVLGPAFLEAHEPTHLNIKYEHEILLGDTVRSEAQIMEDKTIHRIWSGDTLSAEAHIDWTKSEN

SEQ ID NO: 22 - DNA, 735 bp, Artificial sequence
  Synthetic
atgtattcaatgcaacatgaagtgctatattatgaagccgatgtgaccggaaaactgagcctgccaatgatattcaacctggccgtactatcatcaacacaacaatcagtcgacctcggtgtgggacccgattatgcacatgcaaatggagtcggatggataattctacaacatgtcgtggacataaaacgacggccaaaaatcggagaaaaagtggcgctcgaaacactcgcaaaagagttcaacccatttttcgcaaaacgcctatatcgaatcgtcgatgaagcaggaaatgaactcgtgagcatcgatgcgctatatgcaatgatcgacatggaaaaacgaaaaatggcgcgaataccacaagaaatggtcgatgcatatgcgcccgaacgagtgaaaaaaattccgcgacaaccagaacctgatcacatgatcggtgacattccagtcgatgtcgaccaacaatatgccgtgcgatatctggacatcgattcaaatcgccatgtgaacaattcaaaatatttcgattggatgcaagatgttctcggccccgcatttctcgaagcgcatgaaccaacgcacctgaacataaaatatgagcatgaaatactgctgggagacaccgtgcgaagtgaagcgcaaataatggaagataaaacaatacaccgaatatggtccggtgacacgctgagtgctgaagcacacatcgattggacaaaatctgaaaattga

SEQ ID NO: 23 - protein, 249 aa, Clostridium argentinense
MKNIHRENYKVKFNETDYSTKIKMHSLINYMQETSSIHAELLGAGYEELKKHNLFWVVSRLKINMKKYVNWNDEVIVETWPSGVDKMFFTRSFRIYDREENHIGDINAAYLLVAEDSMFPQRISKLPINIPTIENRFEPYERLEKIKFPKDDKVLVAKKKVRYNDIDLNLHVNNAKYIEWVEDCFPLEMYKDMRIETLQLNFIKEAKCGEKIFFYKYNDLEDENTCYIEGIEKQSESQIFQCKLTFNKL

SEQ ID NO: 24 - DNA, 750 bp, Artificial sequence
  Synthetic
atgaaaaacatacaccgagaaaactacaaagtgaagttcaacgaaaccgactacagcaccaaaatcaaaatgcactcgctgataaactacatgcaagaaacatcatcaatacatgcagaacttctcggagccggatatgaagaactgaaaaagcacaacctattttgggtcgtgagccgcctgaaaataaacatgaaaaaatacgtgaattggaatgatgaagtgatcgtggaaacatggccatccggagtggacaaaatgtttttcacgcgatcatttcgaatatatgatcgtgaagaaaaccacatcggagacataaatgctgcataccttctggtcgcagaagattcaatgtttccgcagcgaatatcaaaactgccaataaacataccaacaatcgaaaaccgattcgaaccatatgagcgcctcgaaaaaataaagtttcccaaagatgacaaagtgctcgtcgccaaaaaaaaagtgcgatacaatgacatcgacctgaacctgcatgtgaacaatgcaaaatacatcgaatgggtggaagattgttttccgctggaaatgtacaaagacatgcgaatcgaaacgctgcaactgaatttcataaaagaagccaaatgcggcgagaaaatatttttctacaagtacaacgacctcgaagatgaaaacacatgctacatcgaaggcatcgaaaagcaatccgaatcgcaaatattccaatgcaagctgacattcaacaaactatga

SEQ ID NO: 25 - protein, 240 aa, Lactococcus raffinolactis
MTYKKKYTVPYYETDANGNMKLPSLFNIALQLSGEQSHSLGISDDWLKETYNYAWVVVEYDVTIQRLPRFSEIITMSTFAKSYNKFFCYRDFVFYAENGDTLLTINSTFVLIDTTSRKVAHVEDDIVAPYQSEKISKIVRGHKSTALSDTPLEKSYHVRFNDIDQNGHVNNSKYFDWMTDVLGYDFLSSHVPSRIHLKYSKEVLYGATVTSRVDLVGVQSFHEIVSEGKHAQAEMTWREK

SEQ ID NO: 26 - DNA, 723 bp, Artificial sequence
  Synthetic
atgacatacaaaaaaaaatacaccgtgccatattatgaaaccgatgcaaatggaaacatgaaactaccatcgctattcaacatcgcgctgcaactgagtggagaacaatcgcattcgctcggaatatcagatgattggctgaaagaaacatacaattatgcatgggtggtcgtcgaatatgatgtgacaattcagcgcctgccgcgattttccgaaataataaccatgagcacattcgcaaaatcatacaacaaatttttttgctaccgcgatttcgtattttatgccgaaaacggcgacacgctgctgacaataaattcaacattcgttctgatcgacacaacatcacgaaaagtcgcgcatgtggaagatgacatcgtggcaccataccaatctgaaaaaatatcaaaaatcgtgcgagggcacaaatcaacagcactgagtgacacaccgctggaaaaatcataccatgtgcgattcaatgacatcgaccaaaatggccatgtgaacaattccaaatatttcgattggatgaccgatgtgctcggatatgattttctatcatcgcatgtgccatcgcgaatacacctgaaatattcaaaagaagtgctatatggtgcaacagtgacatcgcgagtcgatctcgtcggtgtgcaatcatttcatgaaatcgtgagtgaaggaaaacatgcacaagccgaaatgacatggcgagaaaaatga

SEQ ID NO: 27 - protein, 139 aa, Petunia integrifolia
MNEFYEVELKVRDYELDQYGVVNNAIYASYCQHCRHELLEKIGVNADAVARNGEALALTEMTLKYLAPLRSGDRFIVKVRISDSSAARLFFEHFIFKLPDQEPILEARGTAVWLNKSYRPVRIPSEFRSKFVQFLRQEA

SEQ ID NO: 28 - DNA, 420 bp, Artificial sequence
  Synthetic
atgaatgaattttatgaagtcgagctgaaagtgcgcgattatgagctggaccaatatggcgtggtgaacaatgcaatatatgcatcatattgccagcattgccgacatgaactgctggaaaaaatcggtgtgaatgccgatgccgtggcacgaaatggtgaagcactcgcgctgaccgaaatgacactgaaatatctggcaccgctgcgaagtggagatcgattcatcgtgaaagttcgaatatcagattcatccgccgcgcgactatttttcgaacatttcatattcaaactgcccgaccaagaaccaatactcgaagcgcgtggaaccgcagtatggctgaacaaatcatatcgccccgtgcgaataccatcagaatttcgaagcaaattcgttcaatttctacgacaagaagcatga

SEQ ID NO: 29 - protein, 244 aa, Peptoniphilus harei
MKIFCKEYEVMNFLSSDGDLKLNHLVSYLIETSNYQSIDLGLSNEKLLDMGYTWMIYKWKIKINRYPRSYEKIKIKTWASGFKNINAFREFEVYCQGEKIIEASAIFLLIDVEKRKAIKIPEVLAEIYGNNGNRIFKSIERVNEPSELEIANRFSYKILRRDLDFNNHVNNSVYLELIYEAVTDEYTHVKFKDINVNYINELKLGDEIVIDFYREEDRFYFFFKSKDQSQIYARICGVSETPIS

SEQ ID NO: 30 - DNA, 735 bp, Artificial sequence
  Synthetic
atgaaaatattttgcaaagaatatgaagtgatgaattttctgagcagcgatggtgacctgaaactgaaccacctggtatcatacctgatcgaaacatcaaattaccaatcaatcgacctcgggctgagcaatgaaaagctgctcgacatgggatacacatggatgatatacaaatggaaaataaagatcaaccgatacccgcgcagctatgaaaaaatcaaaatcaaaacatgggcatccgggttcaaaaacataaacgcatttcgcgagttcgaagtatactgccaaggagaaaaaataatcgaagcatccgcaatatttctgctgatcgatgtcgaaaaacgaaaagcaataaaaattcccgaagtgctggccgaaatatatggaaacaatggaaaccgaatattcaaatccatcgaacgagtgaatgaaccatccgagctcgaaatcgcaaaccgattttcatacaaaatactacggcgtgatctggatttcaacaaccatgtgaacaattctgtatacctggaactgatatatgaagccgtgaccgatgaatacacgcatgtgaaattcaaagacataaacgtgaattacataaacgagctgaagctgggagatgaaatcgtgatcgacttttaccgcgaagaagatcggttttacttttttttcaaatcaaaagaccaatcgcaaatatatgcgcgaatatgtggtgtgagtgaaacgccaatatcatga

SEQ ID NO: 31 - protein, 247 aa, Clostridium botulinum
MVITDKNFEINYHEIDFKKRVLFTTIMNYFEDASLEQSEKLGVGLQYLKENEQAWVLYKWNVTIDRYPEFGEKIIVRTIPLSYRKFYAYRRFQIIDKTGKVIVTGDSIWFLIDINKRRPIKVTEDMQNAYGLSETKEEPFKIDKIKFPEEFHYNNKFKVRYSDIDTNLHVNNVKYISWAIETIPFDIVLNYTLKNFVITYEKEVKYGNDINVYSEMVHNDNNEIVFVHKVENEEGKRVTSAKSIWVK

SEQ ID NO: 32 - DNA, 744 bp, Artificial sequence
  Synthetic
atggtgataaccgacaaaaatttcgaaataaattaccatgaaatcgacttcaaaaagcgcgtgctattcaccaccataatgaactatttcgaggacgcatcgctcgaacaatcagaaaaactcggagtcggcctgcaatatctgaaagaaaatgagcaagcatgggtgctatacaaatggaatgtgacaatcgaccgatacccagagttcggagaaaaaataatcgtgcgaacaattccgctatcataccgaaaattttatgcatatcggcgatttcaaataatcgacaaaaccggaaaagtgatcgtgacaggtgattcaatatggtttctgatcgacataaacaaacggcggccaataaaagtgaccgaagatatgcaaaatgcatatgggctgagcgaaaccaaagaagagccattcaaaatcgacaaaataaaattccccgaagagtttcactacaacaacaaattcaaagtgcgatattccgacatcgacacaaacctgcacgtgaacaatgtgaaatacatatcatgggcaatcgaaacaataccattcgacatcgtgctgaattacacgctgaaaaacttcgtgatcacatacgaaaaagaagtgaaatatggcaacgacataaatgtatactccgaaatggtgcacaacgacaacaatgaaatcgtgttcgttcacaaagtcgaaaatgaagaaggaaaacgtgtgacatcagcaaaatcaatatgggtgaaatga

SEQ ID NO: 33 - protein, 247 aa, Spirochaeta smaragdinae
MKQVSRYTTEHTVMYSETDARGVLSLPSFFALFQEAALLHAEELGFGETYSKQENLMWVLSRLLLEIDAFPKHRDRIRLSTWPKQPQGPFAIRDYILESEEGTVCARATSSWLLLKLDTMRPIRPQTIFANLSMEGIGLAVEGTAPKISEIDNDSKQEMEVTARYSDLDQNNHVNNTRYVRWFLDCYTPEEITTSGNLHFAINYLQAASYSDKLLLRRYDTESDSSVYGYLEDGTPSFSARIERKSD

SEQ ID NO: 34 - DNA, 744 bp, Artificial sequence
  Synthetic
atgaaacaagtgagccgatacacaactgaacacactgtgatgtattccgaaactgatgcacgtggtgtgctgagccttccatcatttttcgcactatttcaagaagccgcactgcttcatgcagaagaactcggattcggtgaaacatattcaaaacaagaaaacctgatgtgggtgctatcgcgcctactactcgaaatcgatgcatttccaaaacatcgtgaccgaatacggctatcaacatggccaaaacagccacaagggccattcgcaattcgagattacatactggaatcagaagaaggaaccgtatgtgcgcgagcaacatcatcatggcttctactgaaactcgacacaatgcgcccaattcgcccgcaaacaatattcgcaaacctgagcatggaaggaatcgggctggctgtcgaaggaacagcgccaaaaatatcagaaatcgacaatgattcaaagcaagaaatggaagtgaccgcgcgatattccgacctcgaccaaaacaaccatgtgaacaacacgcgatatgtgcgatggtttctcgattgctacacgcccgaagaaataacaacatccggaaacctgcatttcgcaataaattacctgcaagccgcatcatattctgacaaacttctgcttcgccgatatgacactgaatccgattcatcagtatatggatacctcgaagatggaacgccatcattttcagcacgaatcgaacgaaaatcagattga

SEQ ID NO: 35 - protein, 245 aa, Eubacterium limosum
MIIYEKKQKINGYECTYNYQLQPTAALNYFQQTSQEQSEQLGVGPEVLDEMGLAWFLVKYKLQFHEYPKFNDEVMVETEAIAFDKFAAHRRFAIKSLDGRMMVEGDTEWMLQNRKENRLERLSNVPELDVYESGHENHFKLKRVAKVEEWTESKNFQVRYLDIDFNSHVNHVKYLAWALETLPLEKVKAGEIETAKIIYKNQGFYGDMITVKSAEIDENTYRMDIENQEGILLCQIEMTMRIRED

SEQ ID NO: 36 - DNA, 738 bp, Artificial sequence
  Synthetic
atgataatatatgaaaaaaagcaaaaaataaatggatacgaatgcacatacaattaccagctgcagcccaccgccgcgctgaattactttcagcaaacatcgcaagaacaatccgaacaactgggtgtcggccccgaagtgctggatgaaatgggactggcatggtttctcgtgaaatacaaactgcaatttcatgaatatccaaaattcaatgatgaagtgatggtcgaaaccgaagcaatcgcattcgacaaattcgcagcgcaccgccgattcgcaataaaatcgctggatggacgaatgatggtggaaggagacactgaatggatgcttcaaaaccgaaaagaaaaccggctggaacgcctatcaaatgtgccagaactcgatgtatatgaatccgggcatgaaaaccatttcaaactgaaacgtgtggcaaaagtggaagaatggactgaatcaaaaaattttcaagtgcgatacctcgacatcgatttcaattcgcatgtgaaccatgtgaaatatctcgcatgggcactggaaacacttccgctggaaaaagtgaaagccggagaaatcgaaacagcaaaaataatctacaaaaaccaaggattttatggagacatgataaccgtgaaatccgccgaaatcgacgaaaacacataccgaatggacatcgaaaaccaagaaggaatactgctatgccaaatcgaaatgacaatgcgaatacgtgaagattga

SEQ ID NO: 37 - protein, 134 aa, Escherichia coli
MNTTLFRWPVRVYYEDTDAGGVVYHASYVAFYERARTEMLRHHHFSQQALMAERVAFVVRKMTVEYYAPARLDDMLEIQTEITSMRGTSLVFTQRIVNAENTLLNEAEVLVVCVDPLKMKPRALPKSIVAEFKQ

SEQ ID NO: 38 - DNA, 405 bp, Artificial sequence
  Synthetic
atgaacacaacgctatttcgatggcccgtgcgagtatattatgaagataccgatgccggaggagtcgtataccatgcatcatatgtcgcattttatgaacgagcgcgaacagaaatgcttcgccaccaccatttttcgcaacaagcgctgatggctgaacgagtcgcattcgtggtgagaaaaatgacagtcgaatattatgcgcccgcgcgcctcgatgacatgctcgaaatacaaaccgaaataacatcaatgcgaggaacatcgctggtattcacacaacgaatcgtgaatgccgaaaacacgctgctgaatgaagccgaagtactggtcgtatgtgtggacccgctgaaaatgaaaccgcgtgcgctaccaaaatcaatcgtcgccgagttcaaacaatga

SEQ ID NO: 39 - protein, 242 aa, Lactococcus lactis
MGIKYQQNYQVPFYESDAFKKMRISSLLAVALQISGEQSTALGRSDVWVFERYGLFWAVIEYELTIHRLPEFNEKITIETEATSYNKFFCYRNFSFLDENGEVLVEIRSTWVLMDKATRKIDRVLDEIVDPYESEKVSKISRPHKFRKIDEFSDAQKIVYPVRFSALDMNGHVNNAKYYDWAADMVDFEFRKSHQPKHVFIKYNHEVLYGEEINALMSWEDEVSHHNFNDGSTQIEIHWGKV

SEQ ID NO: 40 - DNA, 729 bp, Artificial sequence
  Synthetic
atgggaataaaatatcaacaaaattaccaagtgccattttatgaatccgatgcattcaaaaaaatgcgaatatcatcgctgctcgccgtggcgctgcaaatatctggagaacaatcaacagcgctgggacgaagtgatgtatgggtattcgaacgatatggcctattttgggccgtgatcgaatatgaactgacaatacaccgccttcctgagttcaatgaaaaaataaccatcgaaaccgaagccacatcatacaacaaatttttttgctaccgcaacttttcatttctcgatgaaaacggcgaagtgctcgtggaaatacgaagcacatgggtactgatggacaaagcaacgcgaaaaatcgaccgagtactggatgaaatcgtcgatccatatgaatcagaaaaagtgagcaaaatatcgcgcccgcacaaatttcgaaaaatcgatgaattttccgatgcgcaaaaaatcgtataccccgttcgattttccgcgctggacatgaatggacatgtgaacaatgcaaaatattatgattgggccgccgacatggtggatttcgaatttcgaaaatcgcaccagccaaagcatgtattcataaaatacaaccatgaagtgctatatggtgaagaaataaatgcgctgatgagctgggaagatgaagtgagccaccacaatttcaatgatggaagcacgcaaatcgaaatacattggggaaaagtatga

SEQ ID NO: 41 - protein, 246 aa, Clostridium sp.
MLVTDKEYEIHFYEVDYKGRALFTSLMNYFGDISSKQSEDRNMGIDYLKKVNMAWVLYKWNVKIHRYPTYREKVIARTVPYSFRKFYAYRKFYILDIEGNVIVEADSLWFLIDIETRKPVRVQEEMYTGYCLSKDDNEIIDIPKITAPNESDFCKTFDVRYSDIDTNGHVNNSKYISWILEAVPLNIVTQYSLSNLIITYEKETTYGEVIDSCVEVREVDGKAVCKHKIVDKEGNELTVAETTWTR

SEQ ID NO: 42 - DNA, 741 bp, Artificial sequence
  Synthetic
atgctcgtgactgacaaagaatatgaaatacatttttatgaagtcgattacaaagggcgcgcgctattcacatcgctgatgaattatttcggagacatatcatccaagcaatcagaagatcgaaacatgggaatcgattacctgaaaaaagtgaacatggcatgggtgctatacaaatggaatgtgaaaattcatcgatacccaacataccgagaaaaagtgatcgcgcgaaccgtgccatattcatttcgaaaattttatgcataccgcaaattttacattctggacatcgaaggaaatgtgatcgtggaagctgattcgctatggtttctgatcgacatcgaaacgcgaaaaccagttcgagtgcaagaagaaatgtacaccggatattgcctgagcaaagacgacaatgaaataatcgacataccaaaaataaccgcgccaaatgaatccgatttttgcaaaacattcgatgtgcgatattcagacatcgacacaaatggccatgtgaacaacagcaaatacatatcatggattctcgaagccgttccgctgaacatcgtgacgcaatattcactgagcaacctgataataacatatgaaaaagaaacaacatatggagaagtgatcgattcatgtgtggaagtgcgagaagtggatggaaaagccgtatgcaagcacaaaatcgtggacaaagaaggaaatgaactgaccgtggctgaaacaacatggacacgatga

SEQ ID NO: 43 - protein, 136 aa, Haemophilus influenzae
MLDNGFSFPVRVYYEDTDAGGVVYHARYLHFFERARTEYLRTLNFTQQTLLEEQQLAFVVKTLAIDYCVAAKLDDLLMVETEVSEVKGATILFEQRLMRNTLMLSKATVKVACVDLGKMKPVAFPKEVKAAFHHLK

SEQ ID NO: 44 - DNA, 411 bp, Artificial sequence
  Synthetic
atgctcgacaatggattttcatttcccgtgcgagtatattatgaagataccgatgccggaggagtcgtataccatgcgcgatacctgcattttttcgaacgagcacgaaccgaatacctgcgaacgctgaatttcacacaacaaacgcttctggaagaacaacaactggcattcgtggtgaaaacgctcgcaatcgattattgtgtggccgcaaaactcgatgacctgctgatggtcgaaactgaagtgagtgaagtgaaaggagcaacaattctattcgaacaacgcctgatgcgaaacacactgatgctgagcaaagcaaccgtgaaagtcgcatgtgtcgatctgggaaaaatgaaacccgtggcatttccaaaagaagtgaaagccgcatttcaccatctgaaatga

SEQ ID NO: 45 - protein, 243 aa, Weissella paramesenteroides
MRMPHDVVYYEADVTGKLSLPMIYNLAILSSTQQAIDLNIGPEYTHAKGLGWVVLQQLVTINRRPKDGETITLATKAKQFNPFFAKREYRLIDAAGNDLVIMDGLFSMIDMNKRKLARIPKDMAEAYQPEHVRKIPRAPEVTPFDETREADFVQDYFVRYLDIDSNHHVNNSKYAEWMSDVLPVEFLTSHEPTAMNIKYEHEVLYGNKIKSEVQLVDNVTKHRIWFGDVLSAEATIEWTTASN

SEQ ID NO: 46 - DNA, 732 bp, Artificial sequence
  Synthetic
atgcgaatgccgcatgatgtggtatattatgaagctgatgtgaccggaaaactgagccttccaatgatatacaatctcgcaattctatcatcaacgcaacaagcaatcgatctgaacatcggacccgaatacacgcatgcaaaaggcctgggatgggtcgtacttcaacaactggtgacaataaatcggcgcccaaaagatggagaaacaataacgctggcaacaaaagcaaagcaattcaacccatttttcgcaaaacgtgaatatcggctgatcgatgctgctggaaatgatctcgtgataatggatggcctattttcaatgatcgacatgaacaaacgaaaactggcacgaataccaaaagacatggcagaagcataccaacccgaacatgtgagaaaaattccgcgagcacctgaagtgacaccattcgatgaaacacgtgaagccgatttcgtgcaagattatttcgttcgatacctcgacatcgattcaaaccaccatgtgaacaattcaaaatatgcagaatggatgagtgatgtgctgcccgtcgaatttctgacatcgcatgaaccaaccgcaatgaacataaaatacgagcatgaagtgctatatggaaacaaaataaaatccgaagtgcagctcgtcgacaatgtgacaaagcaccgaatatggttcggtgatgtactgagtgctgaagcaacaatcgaatggacaactgcatcaaattga

SEQ ID NO: 47 - protein, 248 aa, Clostridiales bacterium
MFVYEKEYEIHYYEIDYKRRALITSLVDFFGDIATVQSEQLGIGIEYLKENNLAWVLYKWNIDVVKYPLHGEKIIVKTCPYSMKKFYAYRTFEVLNSEGEVIATADSIWFLINIERRRPVRINEDVYRLYGLDYNDQNTLEIEDIKKPDKADLEKIFNVRYSDIDTNQHVNNAKYIAWAIETVPMEVVLNYTIKNLKVIYEKETTYGEIVKVITEIIHNDNTVICIHKIIDKEEKELTLIKTTWEKNF

SEQ ID NO: 48 - DNA, 747 bp, Artificial sequence
  Synthetic
atgttcgtatatgaaaaagaatatgaaatacattactacgaaatcgattacaagcgccgtgcgctgataacatcgctcgtggattttttcggtgacatcgcaacagttcaatctgaacaactgggaatcggaatcgaatatctgaaagaaaacaacctggcatgggtgctatacaaatggaacatcgatgtggtgaaatacccgctgcatggagaaaaaataatcgtgaaaacatgcccatacagcatgaaaaaattttacgcatatcgaacattcgaagttctgaactccgaaggagaagtgatcgcaactgcagattcaatatggtttctgataaacatcgaacgacgacggcctgttcgaataaatgaagatgtataccgactatatggactggattacaatgaccaaaacacgctggaaatcgaagatataaaaaaacccgacaaagccgacctggaaaaaatattcaatgtgcgatattccgacatcgacacaaaccagcatgtgaacaatgcaaaatacatcgcatgggcaatcgaaacagtgccaatggaagtggtgctgaattacaccataaaaaacctgaaagtgatatacgaaaaagaaaccacatacggcgaaatcgtgaaagtgataaccgaaatcatccacaacgacaacaccgtgatctgcatccacaaaataatcgacaaagaagaaaaagagctgacgctgataaaaacaacatgggaaaaaaacttttga

SEQ ID NO: 49 - protein, 245 aa, Streptococcus mitis
MGLTYQMKMKIPFDMADMNGHIKLPDVILLSLQVSGMQSINLGVSDKDVLEQYNLVWIITDYDIDVVRLPQFDEEITIETEALTYNRLFCYRRFTIYDEDGQEIIRMVATFVLMDRDSRKVHPVVPEIVAPYQSEFSKKLVRGPKYTELENAINKDYHVRFYDLDMNGHVNNSKYLDWIFEVMGADFLTNHIPKKINLKYVKEVRPGGMITSSYELNQLESNHQVTSDGDINAQAKIIWQEINTD

SEQ ID NO: 50 - DNA, 738 bp, Artificial sequence
  Synthetic
atgggactgacatatcaaatgaaaatgaaaataccattcgacatggccgacatgaatgggcacataaaacttcctgatgtgatactgctgagcctgcaagtatcaggaatgcaatcaataaatctgggtgtgagtgacaaagatgtgctggaacaatacaacctggtatggataataaccgattatgacatcgatgtggtgaggctaccgcaattcgatgaagaaataacaatcgaaaccgaagcgctgacatacaaccggctattttgctatcggcgattcacaatatatgatgaagatggccaagaaataatacgaatggtcgcaacattcgttctgatggatcgtgattcgcgaaaagttcaccctgtggttcctgaaatcgtcgcgccataccaatccgaattttcaaaaaaactggtgcgagggccaaaatacaccgaactcgaaaatgcaataaacaaagattaccatgtgcgattttatgacctggacatgaacgggcatgtgaacaacagcaaatacctcgattggatattcgaagtgatgggcgccgattttctgacaaaccacattcccaaaaaaataaacctgaaatatgtgaaagaagtgcgcccaggaggaatgataacatcatcatatgagctgaaccagctcgaatcaaaccaccaagtgacatccgatggagacataaatgcgcaagcaaaaataatatggcaagaaataaacactgattga

SEQ ID NO: 51 - protein, 247 aa, Bacteroides finegoldii
MSESNKIGTYKFVAEPFHVDFNGRLTMGVLGNHLLNCAGFHASDRGFGIASLNEDNYTWVLSRLAIELDEMPYQYEDFSVQTWVENVYRLFTDRNFAIMNKEGKKIGYARSVWAMISLNTRKPADLLALHGGSIVDYICDEPCPIEKPSRIKVTNTQPLATLTAKYSDIDINGHVNSIRYIEHILDLFPIDLYKTKRIRRFEMAYVAESYFGDELTFFCDEANENEFHVEVKKNGSEVVCRSKVIFE

SEQ ID NO: 52 - DNA, 744 bp, Artificial sequence
  Synthetic
atgagtgaatcaaacaaaatcggaacatacaaattcgtggccgaaccatttcatgtggatttcaatgggcgcctgacaatgggagtgctgggaaatcatctgctgaattgtgcaggatttcatgcatctgatcgtggattcggaatcgcatcgctgaatgaagataattacacatgggtactgagccggctggcaatcgaactcgatgaaatgccataccaatacgaagatttttccgtgcaaacatgggtggaaaatgtataccggctattcaccgaccgaaacttcgcaataatgaacaaagaaggaaaaaaaatcggatatgcacgaagtgtatgggcaatgatatcactgaacacgcgaaaaccagccgatcttctcgcactgcatggtggaagcatcgtcgattacatatgtgatgaaccatgcccaatcgaaaaaccatcacgaataaaagtgacaaacacgcaaccgctggcaacgctgaccgcaaaatattccgacatcgacataaatgggcatgtgaacagcattcgatacatcgaacacatactggacctatttccaatcgacctatacaaaacaaaacgaatacggcgattcgaaatggcatatgtcgccgaatcatatttcggcgatgagctgacatttttttgcgacgaagccaatgaaaatgaatttcatgtcgaagtgaaaaaaaacggaagcgaagtggtatgccgaagcaaagtgatattcgaatga

SEQ ID NO: 53 - protein, 249 aa, Clostridium sp.
MGISYEKMYEIHYYECDKNLNCTLESIMNFLGDVGNKHAESLNVGMEYLTERNLTWVFYKYNIKINRYPKYEEKIKVKTVAEEFKKFYALRTYEIYDENNIKIVEGSALFLLIDIVKRRAVKITDDQYKAYNVDKGSTGKNLIGRLERLEKVKNNEYVSNFKVRYSDIDFNKHVNNVKYVQWFMDSVPQEIREEYELKEIDILFEHECYYNDEIKCVCEIHKNEDNLLVLSNIQDKDGKELTVFVSKWE

SEQ ID NO: 54 - DNA, 750 bp, Artificial sequence
  Synthetic
atgggaatatcatacgaaaaaatgtatgaaattcactattacgaatgcgacaaaaacctgaattgcacgctggaatccataatgaacttcctcggagatgtgggaaacaaacatgctgaatcactgaatgtcggaatggaatacctgaccgaacgaaacctgacatgggtattctacaagtacaacataaaaataaaccgataccccaaatacgaagagaagatcaaagtgaaaaccgtcgccgaagagttcaaaaaattctacgcgctgcgaacatatgaaatatacgatgaaaacaacatcaaaatcgtcgaaggaagtgcgctatttctgctgatcgacatcgtgaaacgccgagcagtgaaaataaccgatgatcaatacaaagcatacaatgtggacaaaggaagcacaggaaaaaatctgatcgggcgactggaacgcctcgaaaaagtgaaaaacaatgaatatgtgagcaacttcaaagtgcgatactccgacatcgatttcaacaagcatgtgaacaacgtgaaatatgtgcaatggttcatggattcagtgccgcaagaaatacgcgaagaatatgagctgaaagaaatcgacatactattcgagcacgaatgctactacaacgacgaaataaaatgcgtatgcgaaatacacaaaaatgaggacaacctactggttctgagcaacatacaagacaaagatggaaaagaactgactgtattcgtatcaaaatgggaatga

SEQ ID NO: 55 - protein, 141 aa, Solanum lycopersicum
MAEFHEVELKVRDYELDQYGVVNNAIYASYCQHGRHELLERIGISADEVARSGDALALTELSLKYLAPLRSGDRFVVKARISDSSAARLFFEHFIFKLPDQEPILEARGIAVWLNKSYRPVRIPAEFRSKFVQFLRQEASN

SEQ ID NO: 56 - DNA, 426 bp, Artificial sequence
  Synthetic
atggccgaatttcatgaagtcgagctgaaagtgcgagattatgagctggaccaatatggagtcgtgaacaatgcaatatatgcatcatattgccagcatggccggcatgaactgctcgaacgaatcggaatatcagccgatgaagtggcacgaagtggtgatgcactcgcgctgacagaactgagcctgaaatatctggcgccgctgcgaagtggagatcgattcgtcgtgaaagcgcgaatatccgattcatctgccgcgcgactatttttcgaacatttcatattcaaactgccagaccaagaaccaattctggaagcacgtggaatcgccgtatggctgaacaaatcatatcggcctgtgagaataccagctgaatttcgaagcaaattcgtgcaatttctacggcaagaagcatcaaattga

SEQ ID NO: 57 - protein, 396 aa, Picea sitchensis
MYHSPVTNALWHARSSIFERLLDPSVDAPPQSQLLSKTPSQSRTSILYNFSSDYILREQYRDPWNEVRIGKLLEDLDALAGTIAVKHCSDDDSTTRPLLLVTASVDKMVLKKPIRVDTDLKVAGAVTWVGRSSLEIQMVITQPPEGETETGDSVALTANFMFVARDSKTGKSALINRLLPQTEQEKALLAEGEARDMRRKKERQRQGKEFEEGHRLHGDGDRLKALLREGRVLCDMPALADRDSMLIKDTRLENALICQPQQRNLHGRIFGGFLMHRASELAFSTCYAFVGHTPLFLEVDHVDFLRPVDVGDFLRFKSCVLFTQVDDPKRPLIDIEVVAHVTRPELRSSEVSNTFYFTFTVHPVALEGGLKIRKVLPATEEEARHVLERIDAENLN

SEQ ID NO: 58 - DNA, 1191 bp, Artificial sequence
  Synthetic
atgtaccattcgccagtgacaaatgcactatggcatgcgcgaagcagcatattcgaacgacttctggatccatccgtcgatgcgccgccgcaatcacaactgctatcaaaaacgccatcgcaatcgcgaacatcaatactatacaatttttcatccgattacatactgcgtgagcaataccgcgacccatggaatgaagtgcgaatcggaaaactgctggaagatctggatgcgctggcaggaacaatcgctgtgaaacattgcagtgatgatgattcaacaacgcgaccgctacttctggtgactgcatctgtggacaaaatggtgctgaaaaaaccaattcgagtggacactgacctgaaagtggctggtgcagtgacatgggtgggccgaagcagcctggaaattcaaatggtgataacgcaaccgcccgaaggtgaaactgaaactggtgattccgtcgcgctgaccgcaaatttcatgttcgtcgcgcgagattcaaaaaccggaaaatccgcactgataaaccgacttcttccgcaaacagaacaagaaaaagcgctgctggctgaaggagaagcacgagacatgcgacgaaaaaaagaacggcaacgccaaggaaaagagttcgaagaaggccatcgccttcatggtgatggtgatcgcctgaaagcgcttctacgagaaggacgtgtactatgtgacatgcctgcactcgccgatcgtgattcaatgctgataaaagacacacgactggaaaatgcgctgatatgccaaccgcaacaacgaaacctacatgggcgaatattcggtggatttctgatgcaccgtgcatccgaactggcattttcaacatgctatgcattcgtcggacacacaccgctatttctcgaagtggatcatgtcgattttctgcgacccgtggatgtgggagattttctacgattcaaatcatgtgttctattcacgcaagtggatgacccaaaacggccgctgatcgacatcgaagtcgtggcacatgtgacgcggcctgaactacgatcatctgaagtatcaaacacattttatttcacattcacagtgcaccctgtcgcgctcgaaggtggcctgaaaattcgaaaagtgctaccagcaacagaagaagaagcgcgccatgtactcgaacgaatcgatgccgaaaacctgaattga

SEQ ID NO: 59 - protein, 242 aa, Pseudoramibacter alactolyticus
MGKIFERPQAIATYDCLEDHHLSPVAVMNYFQQISLEHSASLKAGPYELSALDLTWIVVKYHVDFWQMPRFLDQLQLGTWASAFKGFTAHRGFFLKNQSGEHMVDGQSHWMMVDRRQNHIVRVNEVPINAVYDVEDQGPRFKMPRLARIKDWENVRQFSVRYLDIDYNGHVNNVCYLAWALACLPAVVLQTRTLKTLDIVFKEQALYGDVVTVKDREIAPNCYRVDIFNANETLLTQLQLQF

SEQ ID NO: 60 - DNA, 729 bp, Artificial sequence
  Synthetic
atgggaaaaatattcgaacgcccgcaagcaatcgcaacatatgattgcctggaagatcatcacctgagcccagtggccgtgatgaattattttcaacaaatatcgctggaacattccgcatcactgaaagccggaccatatgaactatccgcactcgacctgacatggatcgtggtgaaatatcatgtggatttttggcaaatgccacgatttctggaccaacttcaactgggaacatgggcatcagcattcaaaggattcacagcgcaccgaggattttttctgaaaaaccaatctggtgaacacatggtggatggacaatcacattggatgatggtggaccgccggcaaaaccacatcgtgcgtgtgaatgaagtgccaataaatgctgtatatgatgtcgaagatcaaggaccgcgattcaaaatgccgcggctggcacgaataaaagattgggaaaatgtgcggcaattttccgtgcgatacctggacatcgattacaatggccatgtgaacaatgtatgctacctggcatgggcgctggcatgcctacctgccgtggtacttcaaacgcgaacgctgaaaacgctcgacatcgtattcaaagaacaagcgctatatggtgatgtggtgaccgtgaaagaccgagaaatcgcgccaaattgctaccgtgtcgacatattcaatgcaaatgaaacgcttctgacgcaactgcaactacaattttga

SEQ ID NO: 61 - protein, 245 aa, Clostridium botulinum
MVITEKEYEIHYYETHTKHQATITNIIDFFTDVATFQSEKLGVGIDFMMENKMAWMLYKWDINVHRYPKYREKIIVVTEPYAIKKFYAYRKFYILDENRNVIATAKSVWLLIHIEKRKPLKISSEIIKAYNLTDKKSDIKIEKLGKLPEEYTSLEFRVRYSDIDTNGHVNNEKYAAWMLESLPRNIISEYTLINIKITYKKETLYGENIRVLTGIKESEDKLVFIHNVIRENGELLTEGETVWKK

SEQ ID NO: 62 - DNA, 738 bp, Artificial sequence
  Synthetic
atggtgataaccgaaaaagaatatgaaattcactactatgaaacgcacaccaagcaccaagccacaataacaaacataatcgactttttcaccgatgtggcaacatttcaatcagaaaaactgggagtcggaatcgatttcatgatggaaaacaaaatggcatggatgctatacaaatgggacataaatgtgcaccgatacccaaaataccgcgaaaaaataatcgtcgtgaccgagccatatgcaataaaaaaattttacgcataccgcaaattttacattctcgacgaaaaccgaaatgtgatcgcaacagcaaaatccgtatggctgctgattcacatcgaaaaacgaaagccgctgaaaatatcatccgaaataatcaaagcatacaacctgaccgacaaaaaatccgacataaaaatcgaaaagctcggaaaactacccgaagaatacacatcgctggaatttcgagtgagatattcagacatcgacacaaatggacatgtgaacaatgaaaaatatgccgcatggatgctggaatcgcttccgcgaaacataatatccgaatacacgctgatcaacatcaaaatcacatacaaaaaagaaacgctatatggcgaaaacattcgcgtgctgaccggaataaaagaatccgaggacaaactggtattcattcacaatgtgattcgagaaaatggagaacttctgacagaaggtgaaactgtatggaaaaaatga

SEQ ID NO: 63 - protein, 308 aa, Bos taurus
MVLGRGLLGRWSVAELGAVCARLGLGPALLGSLHHLGLRKSLTVDQGTMKVELLPALTDNYMYLLIDEDTKEAAIVDPVQPQKVVETARKHGVKLTTVLTTHHHWDHAGGNEKLVKLEPGLKVYGGDDRIGALTHKVTHLSTLQVGSLHVKCLSTPCHTSGHICYFVTKPNSPEPPAVFTGDTLFVAGCGKFYEGTADEMYKALLEVLGRLPADTRVYCGHEYTINNLKFARHVEPDNTAVREKLAWAKEKYSIGEPTVPSTIAEEFTYNPFMRVREKTVQQHAGETEPVATMRAIRKEKDQFKMPRD

SEQ ID NO: 64 - DNA, 927 bp, Artificial sequence
  Synthetic
atggtactcggacgaggacttctgggacgatggtcagtcgctgaactgggagctgtatgtgcacgactgggactcggacctgcacttctcggaagcctacatcatctcggacttcgaaaatcgctgaccgtcgaccaaggaacaatgaaagtcgaactgctaccagcgctgacagacaattacatgtatctgctgatcgatgaagatacaaaagaagccgcaatcgtcgaccccgttcaaccgcaaaaagtggtcgaaaccgcgcgaaaacatggagtgaaactgacaacagtgctgacaacgcatcaccattgggaccatgctggtggaaatgaaaaactggtgaaactcgaacctggactgaaagtatatggaggtgatgatcgaatcggtgcactgacgcacaaagtgacacatctgagcacactacaagtgggaagccttcatgtgaaatgcctgagcacgccatgccacacatcaggacacatatgctatttcgtgacaaaaccaaattcacctgaaccgccagctgtattcaccggagacacactattcgtggccggatgtggaaaattttatgaaggaaccgctgatgaaatgtacaaagcactactcgaagtactggggcgcctacccgccgacacacgtgtatattgtggacatgaatacacaataaacaacctgaaattcgcgcgccatgtcgaaccagacaacacagccgtgcgagaaaaactcgcatgggcaaaagaaaaatattcaatcggtgaaccaaccgtaccatcaacaatcgccgaagagttcacatacaacccattcatgcgtgtgcgtgaaaaaaccgtgcaacaacatgccggagaaaccgaacctgtggcaacaatgagagcaatacgaaaagaaaaagaccaattcaaaatgccacgtgattga

SEQ ID NO: 65 - protein, 242 aa, Alkaliphilus oremlandii
MTEEFVIPYYDCSGDRFVRPESLLEYMGEASLLHGDTLGVGGADLFKMGFAWMLNRWKVRFIEYPKSRTTITVETWSSGVDRFYATREFNIYDSDRKLLVQASTQWVFCHILKRKPARVPDIISAVYDSEDEHNFYHFHDFKDEVQADEAIEFRVRKSDIDFNHHVNNVKYLNWMLEVLPKQFEDQYLYELDIQYKKEIKQGSLIKSEVSMDIEGEETVCYHKITSNSVLHAFGRSVWKNRK

SEQ ID NO: 66 - DNA, 729 bp, Artificial sequence
  Synthetic
atgactgaagagttcgtgataccatattatgattgcagtggagatcgattcgttcgccctgaatcgctactcgaatacatgggagaagcatcactactacatggtgacacgctgggagtgggaggagcagatctattcaaaatgggattcgcatggatgctgaatcgatggaaagtacgattcatcgaatatccaaaatcgcgaacaacaataactgtggaaacatggtcatctggagtcgaccgattttatgcaacacgagagttcaacatatatgattctgaccgaaaactgctggtgcaagcatcaacacaatgggtattttgccacattctgaaacgaaaacctgcacgagtacctgacataatatccgccgtatatgattccgaagatgagcacaatttttaccattttcatgatttcaaagacgaagtgcaagccgatgaagcaatcgaatttcgagtgcgaaaatctgacatcgatttcaaccaccatgtgaacaatgtgaaatacctgaactggatgctcgaagtgctgccaaagcaattcgaagatcaatacctatacgagctcgacattcaatacaaaaaagaaataaagcaaggaagcctgataaaatccgaagtgagcatggacatcgaaggcgaagaaaccgtatgctaccacaaaataacatcaaattcagtgcttcatgcattcgggcgaagtgtatggaaaaaccgaaaatga

SEQ ID NO: 67 - protein, 251 aa, Desulfotomaculum nigrificans
MYRKEFEVHYYEINQFEEATPVAVLNYLEETAVAHSESVGVGISKLKSQGVAWMLNRWHIKMEKYPLWNEKIVIETWPSRFERFYATREFNIRDSYDHIIGRASSLWVFLNIEKKRPLRIPDKIKDAYGTDPHRAIDEPFGELYNLDDSVEKKEFRVRRSDIDTNNHVNNAKYVDWVLETIPAEIYHNYTLASLEVLYRKEVAFGATIWAGCQGIGKGLNPVYAHSIMNQDGNLALARTMWQRRNKNLHTN

SEQ ID NO: 68 - DNA, 756 bp, Artificial sequence
  Synthetic
atgtatcgaaaagaatttgaagtgcattattatgaaataaatcaattcgaagaagcaacgcccgtcgccgtgctgaattacctggaagaaaccgccgtggcacattctgaatcagtcggagtcggaatatcaaaactgaaatcgcaaggagtcgcatggatgctgaaccgatggcacataaaaatggaaaaatacccgctatggaatgaaaaaatcgtgatcgaaacatggccatcgcgattcgaacgattttatgcaacgcgtgagttcaacatacgagattcatatgaccacataatcgggcgagcatcatcgctatgggtatttctgaacatcgaaaaaaagcgcccactgcgaatacccgacaaaataaaagatgcatatggaaccgatccgcaccgagcaatcgatgaaccattcggagaactatacaacctggatgattccgtggaaaaaaaagaatttcgagtgcggcgaagtgacatcgacacaaacaaccatgtgaacaatgcaaaatatgtggattgggtactcgaaacaattcccgccgaaatataccacaattacacgctcgcatcactggaagtactataccgaaaagaagtcgcattcggtgcaacaatatgggccggatgccaaggaatcggaaaagggctgaacccagtatatgcgcattcaataatgaaccaagatggaaacctcgcgctcgcacgaacaatgtggcaacggcgaaacaaaaatctgcacacaaattga

SEQ ID NO: 69 - protein, 246 aa, Cellulosilyticum lentocellum
MSRLKENYQVDFDVVDFTGKLSINGLCSYMQTVAAKHATKLGINFYKNGEKPTYYWILSRVKYEIDTYPRWEDLVSLETYPGGYEKLFAVRLFDLTDEKGELIGRITGDYLLMDAEKGRPVRIKGATGPLSVLDFPYEGRKIDKIEVPEVVLREQIRKAYYSELDLNGHMNNAHYIRWTVDMLPLEVLKENEIVSLQINYNASITYGVETKLIIGKNEAGNYLVAGNSLDDSVNYFTSEIILRKNK

SEQ ID NO: 70 - DNA, 741 bp, Artificial sequence
  Synthetic
atgagccgcctgaaagaaaattatcaagtcgatttcgatgtcgtggatttcaccggaaaactgagcataaatgggctatgctcatacatgcaaacagtggccgcaaagcatgcaaccaagctgggaataaatttttacaaaaatggcgaaaagccaacatactattggatactgagccgcgtgaaatatgaaatcgacacatacccacgatgggaagatctggtgagcctggaaacatatcctggaggatatgaaaaactattcgctgtgagactattcgacctgaccgatgaaaaaggagaactgatcggccgaataacaggtgattatctactgatggatgccgaaaaaggccgcccagtgagaataaaaggtgcaactggaccgctgagtgtactcgattttccatatgaagggcgaaaaatcgacaaaatcgaagtacccgaagtcgtgcttcgagaacaaattcgaaaagcatattattccgaactggatctgaatggacacatgaacaatgcacattacattcgatggacagtcgacatgcttccactcgaagtgctgaaagaaaacgaaatcgtatcgctgcaaataaactacaatgcatcaataacatacggcgtggaaacaaagctgataatcggaaaaaacgaagccggaaactacctcgtcgctggaaattcgctggatgattctgtgaattatttcacatccgaaataatactgagaaaaaacaaatga

SEQ ID NO: 71 - protein, 244 aa, Paenibacillus sp.
MGNIWTEEHLIYSNEIDYKANCRLSNLLSLMQRAADGDVEHMGGTRDQMVAHHLGWMLTTIDLACERMPIFNETLKITTWNKGTKGPLWLRDFRIFDENNQEIAKACTLWALVDIDKRKVLRPSAYPFNINSNHEDSVGPVPDKLNISDEVELYHSYSITVRYSGIDSNGHLNNSRYADLCMDTLTQSELDTLSILGFHITYYHEVKSAEQIQVLRSDHLEGYIYFRGQSLEDERYFEACLHVG

SEQ ID NO: 72 - DNA, 735 bp, Artificial sequence
  Synthetic
atgggaaacatatggactgaagaacacctgatatattcaaatgaaatcgattacaaagcaaattgccgactgagcaacctactgagcctgatgcaacgagctgcagatggagatgtcgaacacatgggtggaacacgtgaccaaatggtcgcgcaccacctgggatggatgctgacaacaatcgatctcgcatgtgaacgaatgccaatattcaatgaaacgctgaaaataacaacatggaacaaaggaaccaaagggccgctatggctgcgtgattttcgaatattcgacgaaaacaaccaagaaatcgcaaaagcatgcacgctatgggcgctggtggacatcgacaaacgaaaagtactgcgaccatcagcatacccattcaacataaattcaaatcatgaagattccgtgggccctgtgcccgacaagctgaacatatccgatgaagtggaactataccattcatattcaataaccgtgcgatattcaggaatcgattcaaatgggcacctgaacaattcacgatatgcagacctatgcatggacacactgacgcaatcagaactcgacacgctgagcatactcggatttcacataacatattaccatgaagtgaaatcagccgaacaaatacaagtgctgcgaagtgaccacctcgaaggatacatatattttcgtggccaatcactcgaagatgaacgatatttcgaagcatgcctgcatgtcggatga

SEQ ID NO: 73 - protein, 249 aa, Carboxydothermus hydrogenoformans
MIFELEYRIPYYDVDYQKRTLITSLINYFNDIAFVQSENLGGIAYLTQNNLGWVLMNWDIKVDRYPRFNERVLVRTAPHSFNKFFAYRWFEIYDKNGIKIAKANSRWLLINTEKRRPVKINDYLYGIYGVSYENNNILPIEEPQKLLSIDIEKQFEVRYSDLDSNGHVNNVKYVVWALDTVPLEIISNYSLQRLKVKYEKEVTYGKTVRVLTGILSEQKTIVSLHKIVDEDETELCFLESVWFLNEKLS

SEQ ID NO: 74 - DNA, 750 bp, Artificial sequence
  Synthetic
atgatattcgagctggaataccgaataccatattatgacgtggattaccaaaagcgaacgctgataacatcgctgataaattacttcaatgacatcgcattcgttcaatccgaaaacctcggtggaatcgcatatctgacgcaaaacaacctgggatgggtactgatgaattgggacataaaagtggatcgatatccacgattcaatgaacgtgttctggtgagaaccgcaccgcattcattcaacaaatttttcgcataccgatggttcgaaatatacgacaaaaacggaataaaaatcgccaaagcaaattcgcgatggctgctgataaacaccgaaaaacgccgccctgtgaaaataaatgattacctatatggaatatatggtgtgagctatgaaaacaacaacattctgccaatcgaagagccgcaaaaactgctgagcatcgacatcgaaaagcaattcgaagtacgatattccgacctcgattcaaatggccatgtgaacaatgtgaaatatgtggtatgggcactcgacaccgtgccgctcgaaataatatcaaattattcgctgcaacgcctgaaagtgaaatatgaaaaagaagtgacatatggaaaaaccgtgagagtgctgaccggaatactatccgaacaaaaaacaatcgtgagcctgcacaaaatcgtcgatgaagatgaaaccgaactatgctttctcgaatcagtatggtttctgaatgaaaaactatcatga

SEQ ID NO: 75 - protein, 243 aa, Clostridium carboxidivorans
MQYEIQYYEIDCNKKLLLTSLMNYLEDACTMQSEDIGIGLDYMKSKKVAWVLYKWNIHIYRYPLYREKVKVKTIPESFRKFYAYRSFQVFDSRGNIIADASSIWFLINTERRKAMTVTEDMYEAFGLSKEDNKPLSVKKIRKQERVDSEKVFSVRYSDIDTNRHVNNVKYVDWAVETVPLDIVTNCKIVDIIIAYEKETTYGAMIKVLTQIDKKEEGFVCLHKIVDEEDKELALIETLWKNEK

SEQ ID NO: 76 - DNA, 732 bp, Artificial sequence
  Synthetic
atgcaatatgaaattcaatattatgaaatcgattgcaacaaaaagctgctgctgacatcgctgatgaattacctggaagatgcatgcacaatgcaatctgaagatatcggaatcggactcgattacatgaaatcaaaaaaagtggcatgggtgctatacaaatggaacatacacatataccgatacccgctataccgcgaaaaagtgaaagtgaaaaccattcccgaatcatttcgaaaattttatgcataccgatcattccaagtattcgattcgcgtggaaacataatcgccgatgcatcatcaatatggtttctgataaacacagaacgccgaaaagcaatgactgtgacagaagatatgtatgaagcattcgggctgagcaaagaagataacaaaccgctgagtgtgaaaaaaatacgaaaacaagaacgagtcgattctgaaaaagtattttccgtgcgatattccgacatcgacacaaatcgccatgtgaacaatgtgaaatatgtggattgggcagtcgaaacagtaccgctggacatcgtgacaaattgcaaaatcgtcgacatcataatcgcatatgaaaaagaaaccacatatggcgcaatgataaaagtgctgacgcaaatcgacaaaaaagaagaaggattcgtatgccttcacaaaatcgtggatgaagaagataaagaactggcgctgatcgaaacgctatggaaaaatgaaaaatga

SEQ ID NO: 77 - protein, 250 aa, Thermovirga lienii
MEHNFRISYSQAGALGRLKLTGAMNLCQDIADDHAERVGVSVADLLKQSKTWVLHRFKMTIQTMPQRGDLVTIKTWYRPEKNLYSLRNFEMLDCNGKKLLSVQTSWVVVDMNRGRPLRLDRVMPEAYDKNKDENLEVSFQELLLPEKVDVKKTIQVAVTDLDMNFHVNNVHYLRWALDTIPVEILKEYKPKGVEIAFKRPAFYGDSVISEVGIDKNSCSILCRHHIYGEKDGQSMAVISTEWEKISREER

SEQ ID NO: 78 - DNA, 753 bp, Artificial sequence
  Synthetic
atggaacacaattttcgaatatcatattcacaagcaggagcactggggcgactgaaactgactggtgcaatgaatctatgccaagacatcgccgatgatcatgccgaacgtgtgggtgtgagtgtggccgatcttctgaaacaatcaaaaacatgggtgctgcaccgattcaaaatgacaatacaaacaatgccgcaacgtggtgacctggtgacaataaaaacatggtaccggcccgaaaaaaacctatattcgctgagaaatttcgaaatgctggattgcaatggaaaaaagctgctgagtgtgcaaacatcatgggtcgtcgtggacatgaaccgaggccgaccgcttcgcctcgaccgtgtgatgcccgaagcatacgacaaaaacaaagatgaaaacctcgaagtatcatttcaagagctgctgctgccagaaaaagtggatgtgaaaaaaacaattcaagtcgccgtgactgatctcgacatgaattttcatgtgaacaatgttcattacctacgatgggcactggacacaatacccgtggaaattctgaaagaatacaagccaaaaggagtggaaatcgcattcaaacggcccgcattttatggtgattccgtgatatccgaagtcggaatcgacaaaaattcatgcagcattctatgccggcaccacatatatggagaaaaagatgggcaatcaatggctgtgatatcaaccgaatgggaaaaaatatcgcgtgaagaacgatga

SEQ ID NO: 79 - protein, 281 aa, Selaginella moellendorffii
MVYRQTFVVRSYEVGPDKTATLDTFLNLFQETALNHVLISGLAGNGFGTTHEMIRNNLIWVVTRMQVQVERYPAWGNALEIDTWVGASGKNGMRRDWLVRDYKTGSILARATSTWVMMHKDTRRLSKMPDLVRAEISPWFLSRTAFIPEESCSKIEKLDNSNTRYIRSNLTPRHSDLDMNQHVNNVKYLTWMMESLPQNILESHHLVGITLEYRRECSKSDMVESLTHPERGGHLAINGAAAAAAAAAAAPPSQLDFIHLLRMQTGGSEIVRARTSWKSRH

SEQ ID NO: 80 - DNA, 846 bp, Artificial sequence
  Synthetic
atggtataccgacaaacattcgtggtacgatcatatgaagtgggccctgacaaaactgcaacgctggacacatttctgaacctatttcaagaaacagcgctgaatcatgtgctgatatccgggctcgctggaaatggattcggaacaacacatgaaatgattcgaaacaacctgatatgggtggtgacgcgaatgcaagtgcaagtcgaacgatatcccgcatggggaaatgcactcgaaatcgacacatgggtcggagcatcaggaaaaaatggaatgcgccgtgattggctggtgcgtgattacaaaaccggaagcattctcgcacgagcaacatcaacatgggtgatgatgcacaaagacacacgacggctgagcaaaatgcctgacctggttcgagccgaaatatcgccatggtttctgagccgaaccgcattcattcccgaagaatcatgcagcaaaatcgaaaaactcgacaattcaaacacacgatacattcgaagcaacctgacgccacggcattccgatctcgacatgaaccaacatgtgaacaatgtgaaatacctgacatggatgatggaatcgcttccgcaaaacattctcgaatcgcatcatctcgtgggaataacactggaataccggcgtgaatgcagcaaatcagacatggtcgaatcactgacacatccagaacgtggtggacatctcgcaataaatggtgctgcagccgcagcagctgccgcagctgcagcgccaccatcacaactggatttcatacaccttctgagaatgcaaacaggtggaagtgaaatcgtacgagcgcgaacatcatggaaatcacgacattga

SEQ ID NO: 81 - protein, 266 aa, Treponema caldarium
MKALWTEQFTVRTWDVDRNNRLSPSSLFNYFQEVAGNHATELGVGKDALLRGNQAWILSRMTTLLYRRPGWGETITVRTWPRGTEKLFAIRDYDIIDGFGSTIAQGRSAWLLVDVEKLRPLRPQSLTENLPTNTDMPAIPDGAQALTALPELQAAGTRTAAYSDIDYNGHVNNARYIEWIQDILDASILEQTNHFRIDINYLAEIRPQETISLWKEPLPNQDAGTEEHAGERPPFTPFEVTELWAFEGKHIDSGQSSFRAELRCGA

SEQ ID NO: 82 - protein, 801 aa, Artificial sequence
  Synthetic
ATGAAAGCACTATGGACCGAACAATTCACCGTGAGAACATGGGATGTCGATCGAAACAATCGACTATCGCCATCATCGCTATTCAATTATTTTCAAGAAGTCGCCGGAAATCATGCAACAGAACTGGGTGTGGGAAAAGATGCACTACTTCGAGGAAATCAAGCATGGATACTGAGCCGAATGACAACGCTGCTATACCGACGCCCAGGATGGGGTGAAACAATAACTGTGCGAACATGGCCGCGTGGAACAGAAAAACTATTCGCAATACGAGATTATGACATAATCGATGGATTCGGAAGCACAATCGCGCAAGGCCGAAGTGCATGGCTGCTGGTGGATGTGGAAAAACTGCGACCGCTTCGACCGCAATCGCTGACCGAAAATCTGCCAACAAACACTGACATGCCTGCAATACCCGATGGAGCACAAGCACTGACAGCGCTGCCAGAACTACAAGCCGCTGGAACGCGAACTGCTGCATATTCAGACATCGATTACAATGGCCATGTGAACAATGCGCGATACATCGAATGGATACAAGACATTCTCGACGCATCAATACTGGAGCAAACAAACCATTTTCGAATCGACATAAATTACCTCGCCGAAATACGGCCGCAAGAAACAATATCGCTATGGAAAGAACCGCTACCAAATCAAGATGCCGGAACCGAAGAACATGCCGGTGAACGCCCACCATTCACACCATTCGAAGTGACAGAACTATGGGCATTCGAAGGAAAACACATCGATTCTGGACAATCATCATTTCGTGCTGAACTGAGATGTGGTGCATGA

SEQ ID NO: 83 - DNA, 106 bp, Artificial sequence
  Synthetic
tcggtcagtttcacctgatttacgtaaaaacccgcttcggcgggtttttgcttttggaggggcagaaagatgaatgactgtccacgacgctatacccaaaagaaag

SEQ ID NO: 84 - DNA, 1351 bp, Artificial sequence
  Synthetic
ggtctcatatgaaaggaggtatatcgatgttcgaacgtgatattgtggcgacagataacaacaaggcagtcttgcactacccgggcggggagttcgagatggatatcatcgaagcgagcgaaggcaacaacggcgtggtcctgggcaagatgctctccgaaaccggcctgatcaccttcgaccccggttacgtgagcactggcagcaccgagtcgaagatcacctacatcgacggcgatgcgggcatcctgcgctatcggggctatgacatcgccgacctcgcggagaacgccacattcaacgaagtgagctacctcctcattaacggcgagctcccgaccccggacgaactgcacaagttcaacgacgagatccggcatcacacgctgctggatgaggacttcaagtcgcagttcaacgtgttcccccgcgacgcacacccgatggcgaccctggcatcgagcgtgaatatcctgtcgacgtactaccaggaccagctgaatccgctcgacgaagcgcagctggataaggccactgtccgcctcatggcgaaagtcccgatgctggccgcatacgcgcaccgcgcccgcaagggtgccccttacatgtacccggacaactcgctgaacgcgcgcgagaatttcctgcggatgatgttcggctatcccacggaaccgtacgaaatcgacccgatcatggtcaaggccctggacaagctgctgatcctgcacgccgaccacgagcagaattgctccacgtccacggtgcggatgatcggctcggcgcaagccaacatgttcgtcagcatcgcgggcgggatcaacgcgctgtccggccccctccacggcggcgccaaccaagccgtgctggaaatgctggaagatatcaagtcgaaccacggcggcgacgcaaccgagttcatgaataaagtcaagaacaaagaagatggcgtccgtctgatgggcttcggtcatcgcgtctacaagaactacgacccgcgcgcagccatcgtgaaggaaacggcgcacgaaatcctggagcatttgggcggcgacgacttgctggacctggccattaagctcgaagagattgccctggccgacgactactttatcagccgcaagctgtaccccaatgtggacttctataccggcttgatctatcgtgcgatgggcttcccaaccgatttcttcaccgtcctgttcgccatcggccgtctgcccggctggatcgcccattatcgcgagcagctgggggcggcgggtaacaagatcaatcgcccgcgtcaggtgtacaccgggaacgaatcgcgcaaactggtgccgcgcgaagaacggtgatgagagagacc